(19) United States
(12) Patent Application Publication
(10) Pub. No.: US 2016/0128961 A1
(43) Pub. Date: May 12, 2016

(54) DIETARY OMEGA-3 FATTY ACID DERIVED GLYCEROPHOSPHOLIPIDS TO TREAT NEUROPATHIC PAIN

(71) Applicant: LOMA LINDA UNIVERSITY, Loma Linda, CA (US)

(72) Inventors: Marino De Leon, Loma Linda, CA (US); Johnny D. Figueroa, Loma Linda, CA (US)

(21) Appl. No.: 14/896,475

(22) PCT Filed: Jun. 6, 2014
(86) PCT No.: PCT/US2014/041431

§ 371 Date: Dec. 7, 2015

Related U.S. Application Data
(60) Provisional application No. 61/832,675, filed on Jun. 7, 2013.

(51) Int. Cl. A61K 31/202; A61K 35/00; A61K 31/164; A61K 45/06

(52) U.S. Cl. CPC A61K 31/202; A23L 1/3008; A61K 45/06; A61K 35/00; A61K 31/164; A23V 2002/00

(57) ABSTRACT

Disclosed are methods and dietary compositions for the treatment and prevention of neuropathic pain and for pain prognosis. Some embodiments may include the administration of a diet rich in omega-3 fatty acids. Methods for the treatment and prevention of neuropathic pain by administering a diet rich in omega-3 fatty acids, N-acylated ethanolamines, or N-acylated ethanolamine precursors are also provided. Compositions for the treatment and prevention of neuropathic pain are also provided.

### STATEMENT REGARDING FEDERALLY SPONSORED R&D

This invention was made with United States Government support under NIH 5P20MD001632, awarded by the National Institutes of Health. The United States Government has certain rights in this invention. The present disclosure was funded by NIH 5P20MD001632.

### BACKGROUND OF THE INVENTION

1. Field of the Invention

The present disclosure relates to methods and pharmaceutical compositions for treating or preventing neuropathic pain.

2. Description of the Related Art

Chronic neuropathic pain (“CNP”) is a frequent comorbidity following spinal cord injury (“SCI”) and is one of the most important determinants in the perceived quality of life of SCI patients. CNP often fails to respond to conventional pain management strategies. Current CNP therapeutics lack necessary efficacy and are limited in scope by unwanted side effects and poor tolerance.

### SUMMARY OF THE INVENTION

Methods and compositions for the treatment or prevention of neuropathic pain are provided.

In some embodiments, a method for treating or preventing neuropathic pain comprises administering a diet comprising a therapeutically or prophylactically-effect amount of one or more omega-3 polyunsaturated fatty acids. In other variations, a method for treating or preventing neuropathic pain comprises administering an effective amount of an N-acylated ethanolamine precursor or pharmaceutically acceptable derivative thereof. In some embodiments, the N-acylated ethanolamine precursor comprises one or more glycerophospho-containing docosahexaenoyl ethanol amine, glycerophospho-containing docosapentaenoyl ethanolamine, and glycerophospho-containing eicosapentaenoyl ethanolamine.

In some embodiments, a method for treating or preventing neuropathic pain comprises administering an effective amount of an N-acylated ethanolamine or pharmaceutically acceptable derivative thereof. The N-acylated ethanolamine comprises one or more of docosahexaenoyl ethanolamine, docosapentaenoyl ethanolamine, and eicosapentaenoyl ethanolamine in some embodiments.

The described methods may further comprise administering an additional therapeutic or prophylactic agent. In such embodiments, the therapeutic or prophylactic agent may be an opiate, anti-inflammatory agent, or cell.

Further provided is a composition, e.g., a dietary composition, for the treatment or prevention of neuropathic pain. In some embodiments, the composition comprises an N-acrylated ethanolamine compound or pharmaceutically acceptable derivative thereof. In some embodiments, the N-acrylated ethanolamine compound comprises one or more of docosahexaenoyl ethanolamine, docosapentaenoyl ethanolamine, eicosapentaenoyl ethanolamine, glycerophospho-containing docosahexaenoyl ethanolamine, glycerophospho-containing docosapentaenoyl ethanolamine, and glycerophospho-containing eicosapentaenoyl ethanolamine, in a pharmaceutically acceptable carrier.

### BRIEF DESCRIPTION OF THE DRAWINGS

FIG. 1A-B depicts the responsiveness to thermal stimulation in animals receiving control and omega-3 polyunsaturated fatty acid (“O3PUFA”)-enriched diets.

FIG. 2A-B depicts SCI and dietary O3PUFAs modulate the endocannabinoid-related neurometabolome.

FIG. 3A-B depicts the PLS-DA model validation and metabolite impact.

FIG. 4A-B depicts that Chronic O3PUFAs consumption leads to a robust accumulation of diet-derived glycerophospho ethanolamines in the spinal cord.

FIG. 5A-D depicts metabolic features correlated with pain-like phenotypes.

FIG. 6A shows K-means clustering divided animal based on their nociceptive behavior (Δ latency=latency_{endpoint}−latency_{baseline}).

FIG. 6B shows metabolomic analyses using the normal and hyperalgesic clusters confirmed the potential role of the NAE metabolism in pain processing after SCI.

FIG. 6C depicts Basso, Beattie and Bresnahan locomotor scores measured in hyperalgesic and non-hyperalgesic rats.

FIG. 7A-G demonstrates that dietary O3PUFA did not reduce microglial cell immunoreactivity in superficial dorsal horns following chronic SCI.

FIG. 8A-G shows that preventive dietary O3PUFAs reduce the expression of phosphorylated p38 in below-level dorsal horn neurons.

FIG. 9A-J shows that dietary O3PUFA-pretreatment reduces nociceptive fiber sprouting following chronic SCI.

FIG. 10 depicts a timeline outlining experimental design and animal groups.

FIGS. 11A-11B shows that dietary LC-O3PUFAs significantly modulate the non-lipid spinal cord metabolome during acute injury stages.

FIG. 12A-B shows that dietary LC-O3PUFAs significantly modulate the non-lipid spinal cord metabolome during chronic injury stages.

FIG. 13 shows the results of LC-MS/MS data analysis using Ingenuity Pathways Analysis (IPA) software.

FIG. 14 shows the metabolic pathways targeted by dietary LC-O3PUFA in the sham rat spinal cord.

FIG. 15 shows the metabolic pathways targeted by dietary LC-O3PUFA in the injured spinal cord.

### DETAILED DESCRIPTION OF THE PREFERRED EMBODIMENT

Chronic neuropathic pain (CNP) is one of the most important determinants in the perceived quality of life of spinal cord injury (SCI) patients. Unfortunately, current CNP therapeutics lack necessary efficacy and are limited in scope by unwanted side effects and poor tolerance. These shortcomings could be partly overcome with the use of preventive approaches that can provide resilience to damage prior to irreversible biochemical alterations have occurred in the perturbed cord. Trauma to the spinal cord triggers a robust secondary pathophysiological response, leading to cell death, inflammation, and dysfunction. Neuroinflammation is regarded as a hallmark mechanism underlying injury progression and pain processing, and thus represents an attractive target for therapeutic strategies.

Dietary-essential omega-3 polyunsaturated fatty acids (O3PUFAs), such as docosahexaenoic acid (DHA), are integral components of neural membrane phospholipids and play crucial roles in anti-inflammatory responses. Longstanding studies have demonstrated that dietary PUFAs are mediating factors in pain processing, as evidenced by increased threshold for thermal pain and neuropathic pain in rats fed with high dietary omega-3 to omega-6 PUFA ratio. Studies have shown that O3PUFAs and their derivatives can exert strong antinociceptive effects against thermal and chemical stimulation in various animal models. Given this evidence, O3PUFAs may also play important roles in SCI-induced pain.

SCI causes a robust PUFA deregulation and leads to a marked DHA deficiency, which can be associated with impaired recovery and dysfunction. In some embodiments, administration of O3PUFAs maintained the cord PUFA homeostasis, conferred neuroprotection, prevented dysfunction and facilitated recovery after acute and chronic SCI, even when administered in a prophylactic manner. Therefore, in some embodiments, a preventive diet enriched in O3PUFAs can modulate behavioral responses implicated in pathological nociception in rats. Additionally, studies have shown that the diet type at the time of injury can affect pain behaviors associated with nerve lesions. Despite this evidence, diet remains a largely unexplored therapeutic avenue to ameliorate pain in SCI.

The effects of dietary O3PUFAs on thermal pain stimuli in SCI rats can be assess. N-Acylated ethanolamines (NAE) and related endocannabinoids (eCBs) are bioactive lipids implicated in pain processing. To show the effects of dietary O3PUFAs, N-Acylated ethanolamines (NAE) and related endocannabinoids (eCBs) are focused on to identify the involvement of dietary O3PUFAs in the local modulation of these lipids following SCI. Further, the levels of these bioactive lipids can be evaluated to determine whether they are associated with hyperalgesic behaviors and determine the effects of dietary O3PUFAs in the spinal nociceptive system. Deciphering the neurochemical profile that distinguishes pain-like behaviors may have important clinical implications for pain management and allow for improved prognosis in SCI.

### Treatment of Neuropathic Pain

### Materials and Methods

### Animals

Female Sprague-Dawley rats were used and housed in individual cages on alternating 12 h light/dark cycles.

### Diet Composition

Custom AIN-93-based diets were prepared with modifications to the fat composition as described previously. Dietary fats were approximately 6% of the pellets dry weight and were supplied as either soybean oil (control chow) or menhaden fish oil (O3PUFA-enriched chow: DHA=12.82-gm and EPA=6.91-gm per 100 gm of diet). Diets were matched for cholesterol content.

### Surgical and Post-Operative Procedures

Eight weeks after the dietary pretreatment, animals were deeply anesthetized with a mixture of ketamine/xylazine (80 mg/kg and 10 mg/kg, respectively). The spinal cord injuries were generated using the well-characterized New York University (NYU) Impactor. Notably, trauma caused using this device induces below-level pain that is well developed and longstanding, suggesting that the model is suitable for chronic pain research. To produce the contusion, the skin and the muscles overlying the spinal column were cut. A laminectomy was performed at the T9-T10 level and the T8 and T12 spinal processes were clamped to the Impactor, and the exposed dorsal surface of the cord was subjected to weight drop impact using a 10-g rod released from a height of 12.5-mm. Sham animals received only a laminectomy. The animal's body temperature was maintained at 37° C. during the procedure. After operation, muscle layers were sutured and skin layers closed. The bladders of injured rats were expressed using the Crede's maneuver three times a day until voiding reflexes were restored. Cefazolin (Bristol Myers Squibb, New York, N.Y.; 25 mg/kg, s.q.) and Buprenex® (buprenorphine; Reckett and Colman Pharmaceuticals, Inc. Richmond, Va.; 0.05 mg/kg, s.c.) were given to all rats for 5 and 3 consecutive days, respectively. Animals were allowed to survive for 8 or 12 weeks post-operation (wpo) and the spinal cord tissue dissected for metabolomics and immunohistochemical analyses, respectively.

### Nociceptive Testing

Thermal hyperalgesia (TH) was assessed using the well-established Hargreaves withdrawal test to thermal noxious stimulus. This behavior has been found to be a sensitive and reproducible behavioral test to investigate CNP and is exhibited approximately 28 days following contusion injury. In the week prior to the baseline recordings, the animals were habituated to the behavioral testing apparatus by undergoing 5 different daily testing sessions. Once plantar paw placement was re-established, rats were evaluated weekly until animals were euthanized. Briefly, the animals were placed in a Plexiglas enclosure that rested on an elevated glass floor (Plantar Test, UGO BASILE, Biological Research Apparatus, Comerio, Italy). After allowing the animals to acclimate to the chamber for 30 min, a movable focused infrared emitter was placed under the animal's paw. A photocell automatically turned the emitter off when the animal moved its paw and the latency time for the animal to withdraw its paw was recorded. Strength of stimulation was adjusted to produce hindpaw baseline latencies close to 12 seconds (approximately 50-60° C.). A safety cutoff of 20 sec was used to prevent prolonged exposure to the noxious heat. Five different trials were performed per paw with at least 5 min allowed between each trial. The instrument operators were blinded to the treatment assignations. Minimum and maximum latency values were excluded from each paw analysis at each time point.

It is well-recognized that the testing season, the climate (humidity and temperature), the time of day, the cage density, the animal weight, the locomotor behavior, the number of instrument operators, and order of testing have a significant impact on the results of pain studies in rodents. Furthermore, the repetitive nature of the Hargreaves test makes it very susceptible to learning phenomena. Dietary lipids, including DHA, are known modulators of learning and sensitization processes, which could introduce unwanted confounding effects and affect the outcome of sensory results. On the basis of this evidence and to facilitate data interpretation, the thermal latencies are represented as percent change from baseline and were normalized to changes observed in sham animals as previously reported. Briefly, the nociceptive phenotype was determined by the following equation:

$${HWL}_{\%} = ( \frac{{HWL}_{ib} - {HWL}_{ix}}{{HWL}_{ib}} ) - ( \frac{{HWL}_{\overline{sb}} - {HWL}_{\overline{sx}}}{{HWL}_{\overline{sb}}} ) ( \times 100 ) \qquad EQ . 1$$

HWL %=hindpaw withdrawal latency percent change from baseline normalized to sham animals; ib=latency from injured animal in diet A at baseline; ix=latency from injured animal in diet A at time x; sb=averaged latency from all sham animals in diet A at baseline; sx=averaged latency from all sham animals in diet A at time x.

### Metabolomic Profiling

Unbiased metabolic profiling was performed. Animals were deeply anesthetized and transcardially perfused with ice-cold PBS to limit blood contamination. Spinal cord samples (75-100 mg) were flash frozen in liquid nitrogen and immediately stored at −80° C. Samples were homogenized in water at the time of analyses. The protein was precipitated with methanol containing four standards to report on extraction efficiency. The resulting supernatant was split into equal aliquots for analysis on the three platforms. Aliquots were dried under nitrogen and vacuum-desiccated. The metabolomics profiling platform employed for this analysis was based on a combination of three independent platforms: ultrahigh performance liquid chromatography/tandem mass spectrometry (UHPLC/MS/MS²) optimized for basic species, UHPLC/MS/MS² optimized for acidic species, and gas chromatography/mass spectrometry (GC/MS). Controls were analyzed concomitantly with the experimental samples. For instance, aliquots of a well-characterized human plasma pool served as technical replicates throughout the data set, extracted water samples served as process blanks, and a cocktail of standards spiked into every analyzed sample allowed instrument performance monitoring. Experimental samples and controls were randomized across platform run days. The metabolites were identified by automated comparison of the ion features in the experimental samples and compared to a reference library of chemical standard entries that included retention time, molecular weight (m/z), preferred adducts, and in-source fragments as well as associated MS spectra. The neurometabolomics features were curated by visual inspection for quality control using software developed at Metabolon. Archived mass spectrometry data from our previously reported study, which was curated for only identified ‘named’ compounds in Metabolon's chemical reference library, was re-curated to further investigate the unidentified compounds that were detected in the study.

### Metabolomics Analyses

The partial least square-discriminant analysis (PLS-DA) is a supervised method that uses multivariate regression techniques to extract via linear combination of original variables (X) the information that can predict the class membership (Y). This regression was performed using the plsr function provided by R pls package. The classification and cross-validation were performed using the corresponding wrapper function offered by the caret package. To assess the significance of class discrimination, prediction accuracy during training and the separation distance permutation test were performed. In each permutation, a PLS-DA model was built between the data (X) and the permuted class labels (Y) using the optimal number of components determined by cross validation for the model based on the original class assignment. The variable importance in projection (VIP), which is a weighted sum of squares of the PLS loadings and takes into account the amount of explained Y-variation in each dimension was used to measure the impact of each metabolite in the model. Generally, features with high impact have VIP values higher than 1.

### Immunodetection

Immunofluorescence methods have been described previously. Briefly, spinal cord sections were dried at room temperature for 10-15 min, washed with PBS, and post-fixed with 4% PFA for 10 min. The sections were blocked and incubated at 4° C. ON in 20% normal donkey serum with 0.1% Tween-20 with rabbit anti-phosphorylated-p38, p-p38 (1:200; R&D Systems, Minneapolis, Minn.) and mouse anti-NeuN monoclonal antibody (1:250; Millipore, Billerca, Mass.). Additional experiments used mouse anti-CD11b (OX42, 1:100; AbD Serotec, Raleigh, N.C.) to examine reactive microglial cells. Alternatively, sections were incubated with rabbit anti-GAP43 (1:500; Abcam, Cambridge, Mass.) and sheep polyclonal calcitonin gene-related peptide (CGRP; 1:500; Abcam). The sections were then washed with PBS and incubated in secondary antibodies [Alexa Fluor® 594-conjugated donkey anti-rabbit or donkey anti-sheep (1:500; Invitrogen, Carlsbad, Calif.) and Alexa Fluor® 488-conjugated donkey anti-mouse or donkey anti-rabbit (1:500; Invitrogen)]. Primary antibody omission and normal serum controls were used to confirm the specificity of the immunoreaction. Slides were examined with an Olympus Optical Fluoview FV1000 confocal microscope. Unbiased stereological methods were followed as previously reported. Two blinded observers quantified the immunoreactivity in lamina I to III, which were identified by superimposing photomicrographs with spinal cord diagrams from the Watson, Paxinos, and Kayalioglu spinal cord atlas. For each animal, the p-p38-positive neurons were counted manually in at least 4 randomly selected areas of the superficial dorsal horns. The mean number of p-p38-expressing neurons was then tabulated for each animal and group. For fiber sprouting analyses, the CGRP-GAP43 double labeling immunoreactivity was quantitated by inverting merged images into black (marker-positive) and white in the NIH Image J program for measurement of positive pixels/area in the dorsal horns laminae I to III. Results were obtained by averaging measurements made by blinded investigators.

### Statistical Analysis

Statistical analyses were performed using SPSS version 20.0 (IBM: SPSS, Armonk, N.Y.), Prism 5 software v5d (GraphPad Software Inc., San Diego, Calif.), the “R” program (http://cran.r-project.org/), and metaboanalyst. Two-Way Analysis of Variance (ANOVA) followed by Bonferroni post-hoc comparisons was used to determine the effect of the diet type, injury, and time on hindpaw thermal withdrawal latencies and differences within and between groups. To determine the antinociceptive effects of dietary O3PUFAs we calculated the area under the nociception versus time curve (AUC) and subjected AUCs to student's t-tests. ANOVA contrasts were used to identify features that differed significantly between tested groups. All other data were assessed by Mann-Whitney U test. The Kolmogorov-Smirnov and Shapiro-Wilk normality tests together with the Grubbs' method, also known as ESD (extreme studentized deviate; www.graphpad.com), were used to investigate outliers and spread. Spearman's rank correlation tests were used to explore associations between detected metabolites and the sensory phenotype. Data are presented as mean±SEM. Statistical differences were considered significant at p<0.05 unless otherwise specified.

### Results

### General Conditions

Experimental spinal cord injury (SCI) leads to a marked docosahexaenoic acid (DHA) deficiency and motor and autonomic deficits, which were corrected by dietary omega-3 polyunsaturated fatty acids (O3PUFAs) prophylaxis. Dietary intervention can reduce thermal hypersensitivity in SCI. The antihyperalgesic effects of this dietary strategy were characterized and the extent to which dietary O3PUFAs impact the levels of bioactive metabolites and cellular targets associated with nociception and inflammation in the injured cord were investigated.

Preventive Dietary O3PUFAs Attenuate the Development of Below-Level Thermal Hyperalgesia after Injury to the Spinal Cord

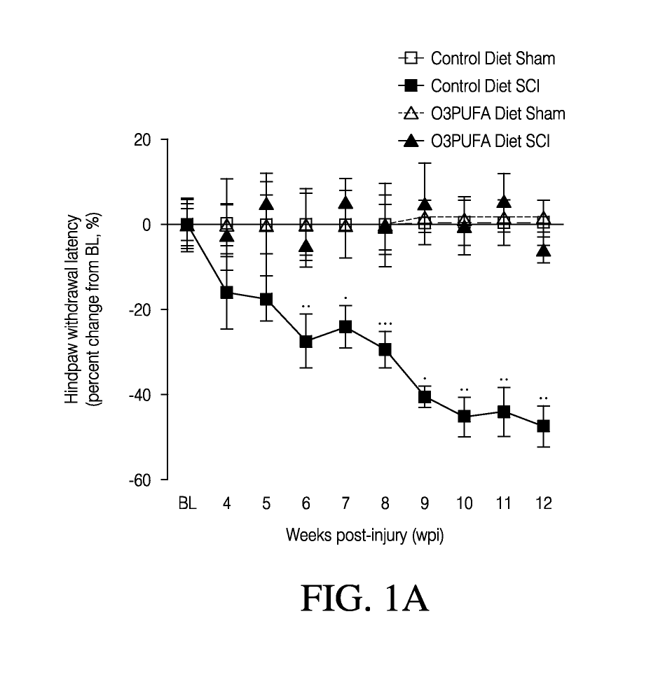

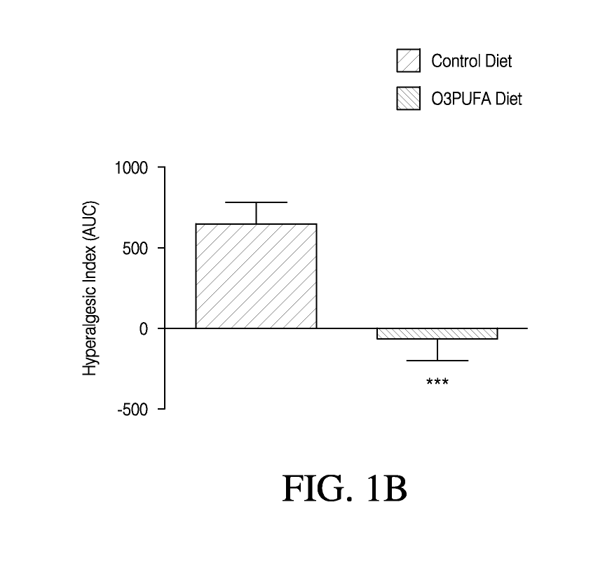

To examine the effect of dietary O3PUFAs on the onset and maintenance of neuropathic pain after SCI, Hargreaves testing can be used. This testing can include testing the paw thermal sensitivity to noxious heat. No differences in the average baseline hindpaw withdrawal latencies were found between groups (10.61±0.59 s for control-fed animals and 11.34±0.44 s for animals receiving O3PUFA diets; mean±SEM, p>0.05). Two-way ANOVA can be used to identify the diet type and operation as significant sources of variation [for the diet effect F(3,49253)=40.22, p=0.0001, n=8-18 rats per group]. When compared to individual baseline latencies, post hoc revealed a significant reduction in the thermal thresholds of rats consuming control diets at 6 wpi (p<0.05). This pain-like behavior was maintained until the completion of the study at 12 wpi. It was shown that the animals consuming diets rich in O3PUFAs showed no significant alterations in their hindpaw withdrawal latencies when compared to their individual baseline values as illustrated in FIG. 1A (p>0.05).

Post hoc comparisons between diet groups revealed that the most significant behavioral differences occurred between 8 and 12 wpi and hence this period can be the focus period for the observation and testing (p<0.01). Sham-operated rats did not show significant changes in their thermal thresholds when compared to their individual baseline values (p>0.05).

Calculation of the area under the thermal withdrawal latency change versus time curve (AUC) showed a potent antihyperalgesic effect of dietary O3PUFAs in SCI rats as shown in FIG. 1B (Mann Whitney U rank test p=0.0008; n=18).

FIG. 1A-B depicts the responsiveness to thermal stimulation in animals receiving control and omega-3 polyunsaturated fatty acid (“O3PUFA”)-enriched diets. FIG. 1A shows that the thoracic contusion to the spinal cord leads to below-level thermal hyperalgesia in animals receiving control diets. Hindpaw withdrawal latencies (averaged percent change from baseline) are plotted versus time (weeks post-injury, wpi). For each timepoint, the individual latencies were adjusted to the percent change from baseline observed in sham animals receiving the same diet using equation 1 as described in the Materials and Methods section. No significant latency alterations were observed in sham animals. Notably, dietary O3PUFAs prevented the development of thermal hyperalgesia (p>0.05 when compared to individual baseline values). TW-ANOVA identified the diet type and surgery as significant sources of variation [for diet/surgery F(3,49253)=40.22, p=0.0001, n=8-18]. Bonferroni's post hoc testing showed significant latency changes from baseline in the animals fed with control diets from 6-12 wpi (p<0.05). Asterisks represents the significance level after post hoc testing: (*)=p<0.05; (**)=p<0.01; (***)=p<0.001.

To investigate the overall effect of O3PUFA in thermal hindpaw sensitivities, the hyperalgesic Index (HI) was generated using the area under the curve (AUC) as shown in FIG. 1B. Analyses of the AUC revealed that the O3PUFA diet had a significant antihyperalgesic effect (Mann Whitney U rank test; p<0.001). Each bar represents mean±SEM; n=18.

Metabolomic Profiling Reveals Distinctive Endocannabinoid Signatures Associated with Chronic SCI and Dietary O3PUFAs

An untargeted metabolomics approach can be used to investigate the neurochemical consequences of O3PUFAs consumption on the endocannabinoid (eCB) metabolome. The eCB metabolome can be expanded to include the ethanolamines, glycerides, and metabolic precursors, intermediates, and derivatives. These metabolites have been implicated in regulating anti-inflammatory responses, but whether dietary PUFAs impact the levels of these bioactive lipids in SCI has not been comprehensively evaluated. To address this issue, both LC/MS and GC/MS-based metabolomics were employed on cord samples collected from sham and contusion SCI operated Sprague-Dawley rats that received either control or O3PUFA-enriched diets.

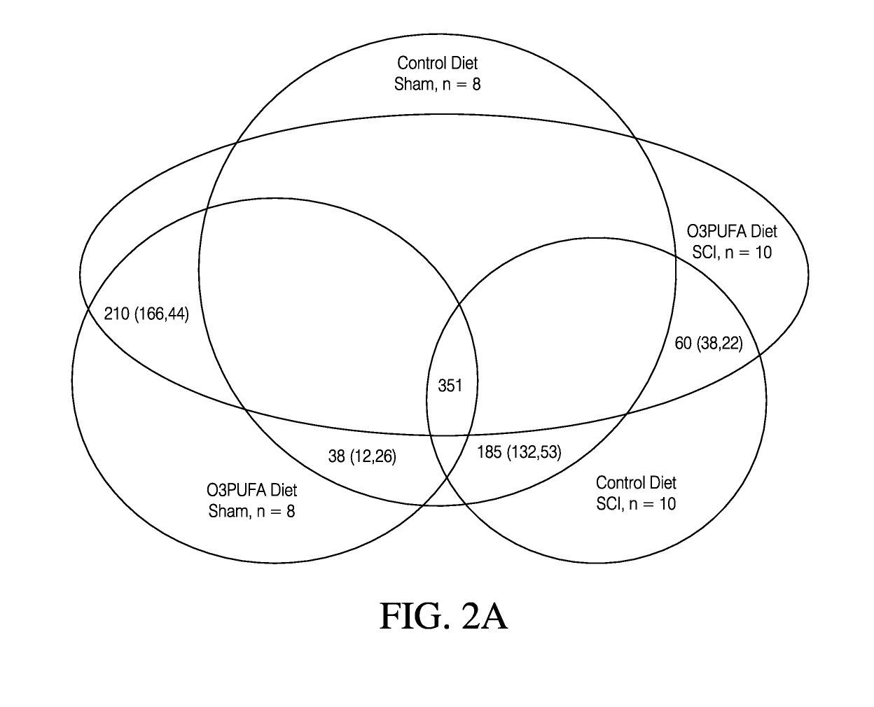

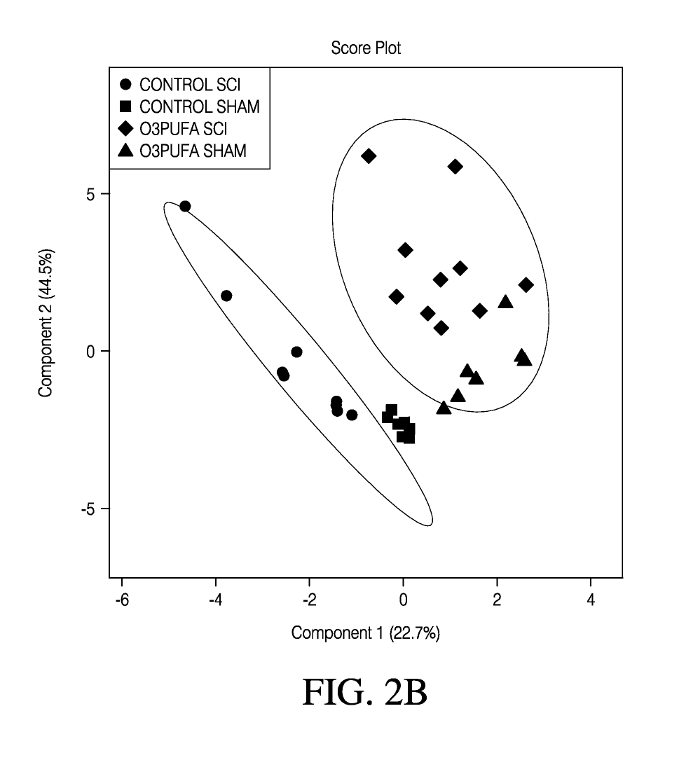

FIG. 2A-B show that SCI and dietary O3PUFAs modulate the endocannabinoid-related neurometabolome. FIG. 2A is a Venn diagram depicting the numerical interactions among data sets. ANOVA contrasts analyses were used to evaluate the regulation pattern differences between groups. The total number of features detected across 36 spinal cord tissue samples was 351 metabolites. The number of total metabolites was significantly altered between groups (e.g., the contrast between O3PUFA diet SCI and Control diet SCI revealed 60 significantly altered metabolites; 38 upregulated and 22 downregulated; p<0.05). FIG. 2B shows a partial least square discriminant analysis (PLS-DA) distinguished subgroups based on dietary intake at 8 weeks post-injury (wpi). A model was constructed using scaled intensity peaks of the detected features associated with the endocannabinoids (eCBs) system: classic eCBs, eCBs glycerols, and related N-acyl ethanolamines (NAEs) and metabolites. Projections provided statistically significant separations between subgroups.

FIG. 2A summarizes the four groups analyzed in this study and the metabolomic interactions between them. A total of 275 named metabolites and 76 unnamed biochemicals were detected and analyzed. The diet rich in O3PUFA significantly changed more than 20% of the detected metabolic features (74 total altered features: 40 upregulated, 34 downregulated, when compared to animals receiving control diets). More than 40% of these altered metabolites were associated with the endocannabinoid metabolome.

The partial least square-discriminant analysis (PLS-DA) score plot was obtained using the variation scores of the first two principal components, component 1 (22.7%) and component 2 (44.5%). As shown in FIG. 2B, these analyses revealed distinctive endocannabinoid-related profiles between groups. Each plot mark corresponds to an animal subject and the variability in relative metabolite levels detected for that animal. Hotelling's T² confidence ellipse, at a significance level of 0.05, showed no outliers.

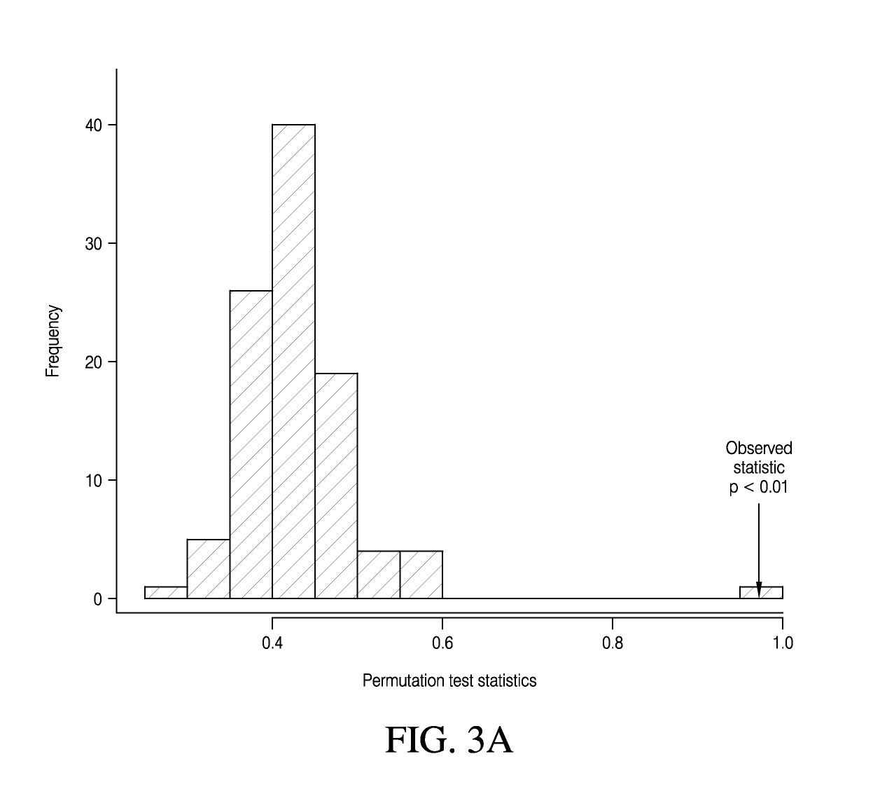

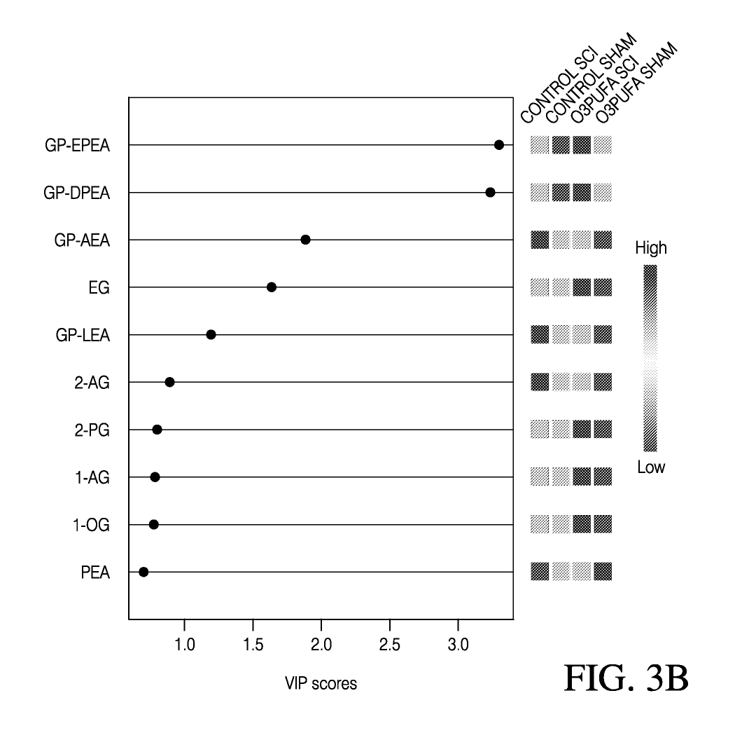

FIG. 3A-B depicts the PLS-DA model validation and metabolite impact. A prediction accuracy training permutation test validates the PLS-DA model by showing a significant observed statistic (p<0.01) as shown in FIG. 3A. The variable influence on projection (VIP) analyses, which reflect the relative importance of metabolites showed the significant contribution of selective NAEs, endocannabinoids, endocannabinoid glycerols, and O3PUFA-derived glycerophospho ethanolamines (GP-NAEs) in the PLS model as shown in FIG. 3B. The boxes on the right indicate the relative concentrations of the corresponding metabolite in each group under study. Abbreviations include: GP, glycerophospho; EPEA, eicosapentaenoyl ethanolamine; DPEA, docosapentaenoyl ethanolamine; AEA, arachidonoyl ethanolamine; EG, eicosenoyl glycerol; LEA, linoleoyl ethanolamine; 2-AG, 2-arachidonoyl glycerol; 2-PG, 2-palmitoyl glycerol; 1-OG, 1-oleoyl glycerol; PEA, palmitoyl ethanolamine.

Permutation analyses validated the class discrimination and neurometabolomic separation (observed test statistic p<0.01). Consequently, more than 67% (component 1+component 2) of the metabolomics differences can be explained with certainty by the generated PLS model. For simplicity, only the prediction accuracy during training result is shown in FIG. 3A.

Both chronic SCI and the preventive diet enriched in O3PUFAs had a significant impact in the levels of acyl glycerol class endocannabinoids and in the metabolism of N-acyl ethanolamines as shown in FIG. 3B. Also, the cord of injured animals receiving the O3PUFAs showed higher levels of eicosenoyl, palmitoyl, arachidonoyl, and oleoyl glycerols when compared to control fed injured animals. In general, the diet rich in O3PUFAs skewed the metabolomic profile towards increased levels of long-chain N-acyl ethanolamines. In particular, a selective group of glycerophospho-containing N-acyl ethanolamines (GP-NAEs) were identified. These molecules showed the strongest influence (highest variable importance in projection, VIP, values) to the observed metabolomics differences between groups.

### Dietary O3PUFA Leads to a Marked Accumulation of Diet-Derived N-Acyl Ethanolamine (NAEs) Precursors

The beneficial neurological effects of O3PUFAs are partly related to their anti-inflammatory properties, however the exact mechanisms behind these actions are being considered and mechanisms of action are described in more detail herein. A putative mechanism could be via their conversion to related N-acyl ethanolamines (NAEs). NAEs are a large class of long-chain signaling lipids implicated in diverse physiological processes, including inflammation, nociception, cognition, anxiety, and appetite. Notably, these fatty amides can exert cannabimimetic actions as endogenous agonist of cannabinoid receptors. The evidence demonstrates the significant impact of diet in the regulation of these bioactive lipids after chronic SCI.

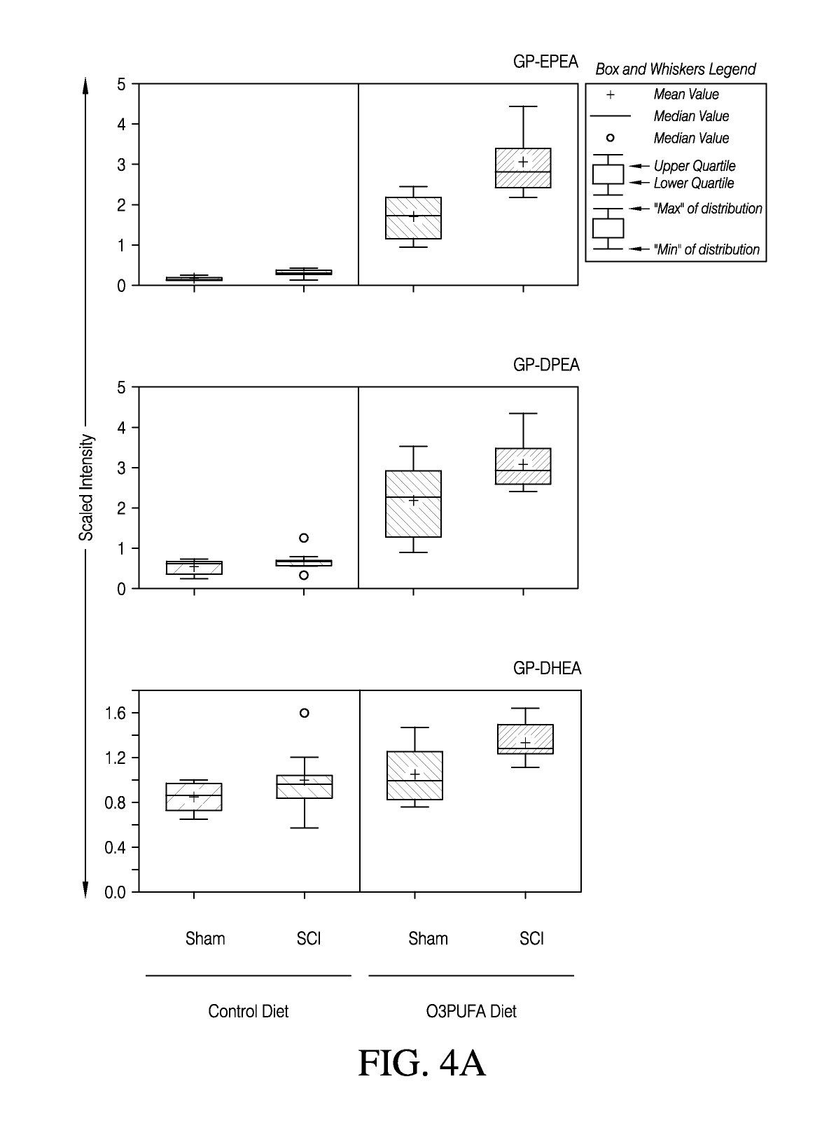

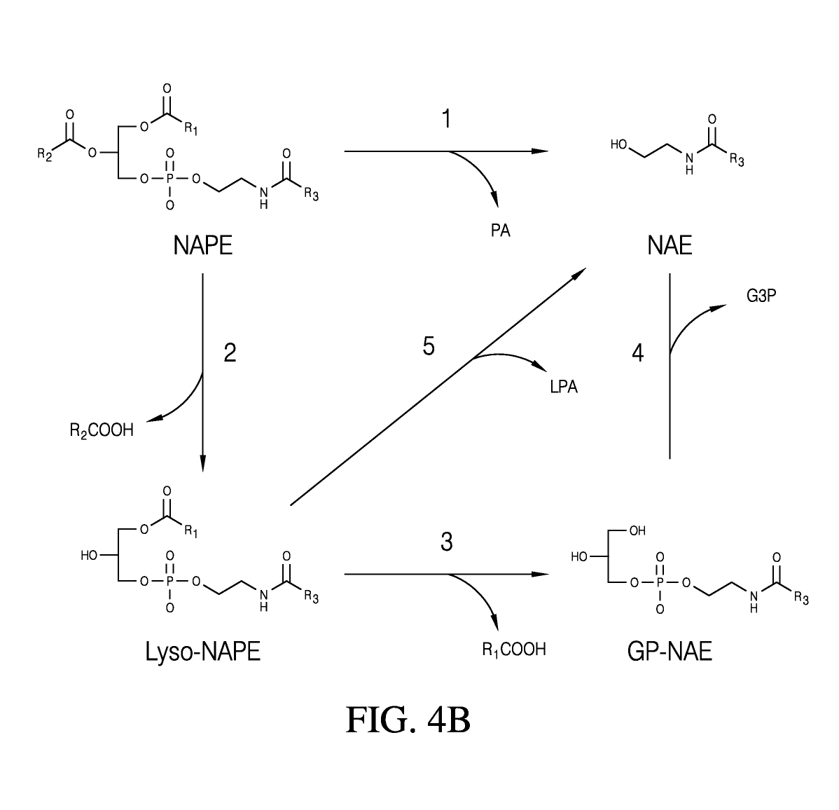

FIG. 4A-B depicts that chronic O3PUFAs consumption leads to a robust accumulation of diet-derived glycerophospho ethanolamines in the spinal cord. Box and whiskers graphs, as shown in FIG. 4A, illustrate a marked increase in the levels of O3PUFA-dervied GP-NAEs (relative to the median metabolite levels in each group). FIG. 4B shows the potential metabolic pathways for the biosynthesis of NAEs (Adapted and modified from Simon GM, Cravatt BF (2008) Anandamide biosynthesis catalyzed by the phosphodiesterase GDE1 and detection of glycerophospho-N-acyl ethanolamine precursors in mouse brain, J Biol Chem. pp. 9341-9349 and Simon GM, Cravatt BF (2006) Endocannabinoid biosynthesis proceeding through glycerophospho-N-acyl ethanolamine and a role for alpha/beta-hydrolase 4 in this pathway. J Biol Chem. pp. 26465-26472, both of which are herein incorporated by reference in their entirety). Reaction 1 is mediated by an NAPE-selective phospholipase D (PLD). Pathways 2-3-4 and 2-5 are NAPE-PLD independent. Recent studies suggest the involvement of a novel phospholipase A/B, named Abh4, α-β-hydrolase 4 in the NAPE conversion to GP-NAE (reaction 2 and 3). The secretory PLA2 can also release fatty acid from sn-2 position of NAPE (reaction 2). Reaction 4 is catalyzed by a new glycerophosphodiesterase, GDE1. Lyso-PLD catalyzes reaction 5. Abbreviations include: DHEA, docosahexaenoyl ethanolamine; DPEA, docosapentaenoyl ethanolamine; EPEA, eicosapentaenoyl ethanolamine; GP, glycerophospho, NAE, N-acyl ethanolamine; NAPE, N-acyl phosphatidyl ethanolamine; G3P, glycerol-3-phosphate; LPA, lyso-phosphatidic acid; PA, phosphatidic acid.

Dietary O3PUFAs lead to a robust accumulation of N-acyl ethanolamines glycerophospholipids containing DHA, DPA, and EPA fatty acids as shown in FIG. 4A. Interestingly, metabolomic analysis revealed very low abundance of these lipids in the cord of animals receiving control diets.

FIG. 4B illustrates the current knowledge on the NAEs biosynthetic pathways (adapted and modified from Simon GM, Cravatt BF (2008) Anandamide biosynthesis catalyzed by the phosphodiesterase GDE1 and detection of glycerophospho-N-acyl ethanolamine precursors in mouse brain, J Biol Chem. pp. 9341-9349 and Simon GM, Cravatt BF (2006) Endocannabinoid biosynthesis proceeding through glycerophospho-N-acyl ethanolamine and a role for alpha/beta-hydrolase 4 in this pathway, J Biol Chem. pp. 26465-26472, both of which are herein incorporated by reference in their entirety). It has been proposed that NAEs are biosynthesized from their corresponding N-acyl phosphatidyl ethanolamines (NAPEs). This can occur through a single NAPE-PLD-dependent pathway (NAPE-PLD). Alternatively, NAPE-PLD-independent multi-step processes have been recently reported and involve alpha/beta-hydrolase 4 (ABDH4 or Abh4) and the glycerophosphodiesterase, GDE1. The results presented herein suggest a marked activation of NAPE-PLD-independent pathways following chronic SCI.

The effects of the diet and chronic SCI on the detected metabolites associated with the endocannabinoid and related NAEs are summarized in Table 1. ANOVA contrasts were performed to determine statistical differences in metabolite relative amounts between groups (differences were considered significant when p<0.05; n=at least 8 rats per group).

Table 1 depicts that the endocannabinoid (eCB) metabolome is altered following chronic SCI and influenced by dietary O3PUFAs. ANOVA contrasts were performed to determine statistical differences in metabolite relative amounts (differences were considered significant when p<0.05; n=8 rats per sham group and 10 rats per injury group). Comparisons were made between the four studied groups: (1) sham control diet, (2) SCI control diet, (3) sham O3PUFA-rich diet, and (4) SCI O3PUFA-rich diet. Notably, in spinal cord injured animals, the O3PUFA diet decreased the levels of the glycerophospho 2-LEA and 2-AEA and dramatically increased the levels of O3PUFA-derived GP-NAEs. Numbers represent fold of change. Bold: indicates significant difference (p≦0.05) between the groups shown, metabolite ratio of <1.00 or narrowly missed statistical cutoff for significance 0.05<p<0.10, metabolite ratio of <1.00. Italics: indicates significant difference (p≦0.05) between the groups shown; metabolite ratio of ≧1.00 or narrowly missed statistical cutoff for significance 0.05<p<0.10, metabolite ratio of ≧1.00. No Format: mean values are not significantly different for that comparison. Abbreviations: LEA, linoleyl ethanolamine; AEA, arachidonoyl ethanolamine; GP-NAEs, glycerophospho n-acyl ethanolamines.

TABLE 1

| | | Fold of Change | | | |
|---|---|---|---|---|---|
| | | ANOVA Contrasts | | | |
| | | | O3PUFA- | O3PUFA- | O3PUFA- |
| | | Ctrl-SCI | SCI | Sham | SCI |
| | | Ctrl- | O3PUFA- | Ctrl- | Ctrl- |
| | Biochemical Name | Sham | Sham | Sham | SCI |
| PUFAs | eicosapentaenoate (EPA; 20:5) | 5.09 | 4.08 | 6.71 | 5.37 |
| | docosahexaenoate (DHA; 22:6) | 2.87 | 3.30 | 1.20 | 1.38 |
| | arachidonate (AA; 20:4) | 1.14 | 1.03 | −1.14 | −1.25 |
| eCBs | oleic ethanolamide (OEA) | −1.41 | −1.33 | −1.03 | 1.02 |
| | palmitoyl ethanolamide (PEA) | −1.92 | −1.61 | 1.05 | 1.24 |
| | 2-arachidonoyl glycerol (2-AG) | 2.21 | 2.37 | −1.18 | −1.10 |
| | 2-oleoylglycerol (2-OG) | 1.66 | 2.07 | −1.30 | −1.03 |
| Precursors, | ethanolamine | 1.34 | 1.69 | 1.08 | 1.36 |
| Derivatives, | phosphoethanolamine | 1.92 | 2.47 | −1.19 | 1.08 |
| Isomers, | glycerophosphoethanolamine | 1.12 | 1.93 | −1.04 | 1.64 |
| Candidates | glycerol | 1.02 | 1.01 | −1.04 | −1.04 |
| | glycerol 3-phosphate (G3P) | 1.41 | 1.90 | 0.94 | 1.26 |
| | 1-palmitoyl glycerophosphoethanolamine | 1.60 | 1.18 | 1.38 | 1.01 |
| | 2-palmitoyl glycerophosphoethanolamine | 1.10 | −1.04 | 1.05 | −1.09 |
| | 1-palmitoleoyl glycerophosphoethanolamine | 3.26 | 3.77 | 1.20 | 1.38 |
| | 2-palmitoleoyl glycerophosphoethanolamine | 1.43 | 1.44 | 1.24 | 1.25 |
| | 1-stearoyl glycerophosphoethanolamine | 1.59 | 1.40 | 1.22 | 1.07 |
| | 1-oleoyl glycerophosphoethanolamine | 2.33 | 2.36 | 1.08 | 1.10 |
| | 2-oleoyl glycerophosphoethanolamine | −1.12 | −1.20 | 1.10 | 1.02 |
| | 2-linoleoyl glycerophosphoethanolamine | 1.35 | 1.02 | −1.47 | −1.96 |
| | 1-arachidonoyl glycerophosphoethanolamine | 4.52 | 4.06 | −1.15 | −1.28 |
| | 2-arachidonoyl glycerophosphoethanolamine | 1.20 | 1.06 | −1.16 | −1.32 |
| | 2-docosapentaenoyl | 1.26 | 1.42 | 4.05 | 4.56 |
| | glycerophosphoethanolamine (GP-DPEA) | | | | |
| | 2-docosahexaenoyl | 1.18 | 1.28 | 1.24 | 1.34 |
| | glycerophosphoethanolamine (GP-DHEA) | | | | |
| | eicosapentaenoyl | 1.83 | 1.8 | 10.27 | 10.15 |
| | glycerophosphoethanolamine (GP-EPEA) | | | | |
| | 1-palmitoyl plasmenylethanolamine | 2.28 | 1.78 | 1.48 | 1.15 |
| | 1-palmitoyl glycerol (1-monopalmitin) | 2.53 | 2.53 | 1.10 | 1.10 |
| | 2-palmitoyl glycerol (2-monopalmitin) | 1.89 | 2.27 | −1.27 | −1.04 |
| | 1-stearoyl glycerol (1-monostearin) | 1.94 | 2.11 | 1.01 | 1.10 |
| | 1-oleoyl glycerol (1-monoolein) | 2.42 | 2.58 | −1.03 | 1.04 |
| | 1-arachidonyl glycerol | 1.70 | 2.64 | −1.56 | −1.01 |
| | eicosenoyl glycerol (monoeicosenoin) | 3.70 | 3.22 | −1.06 | −1.22 |

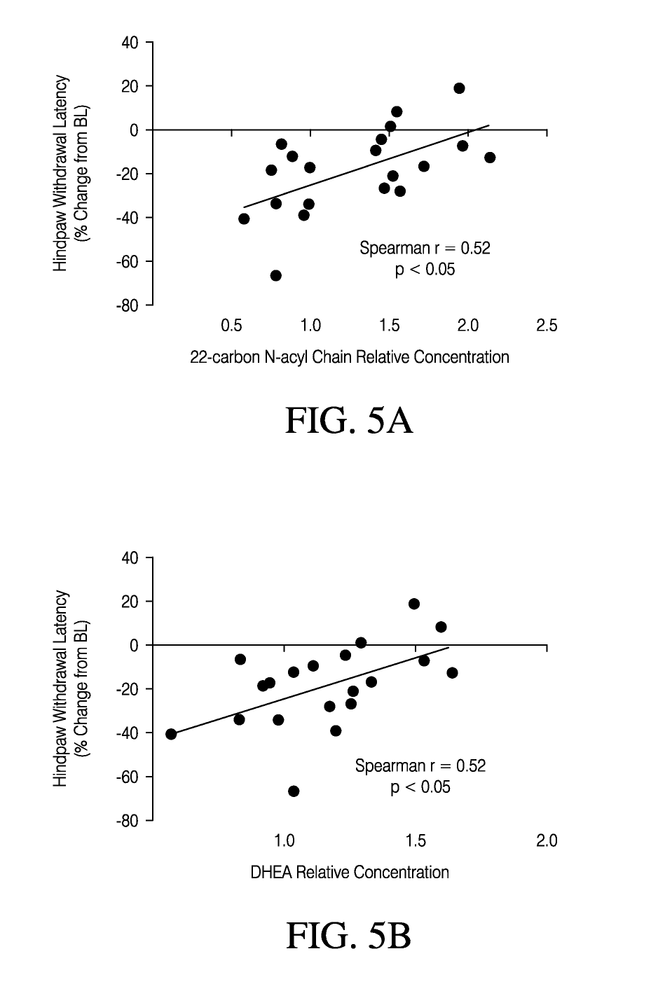

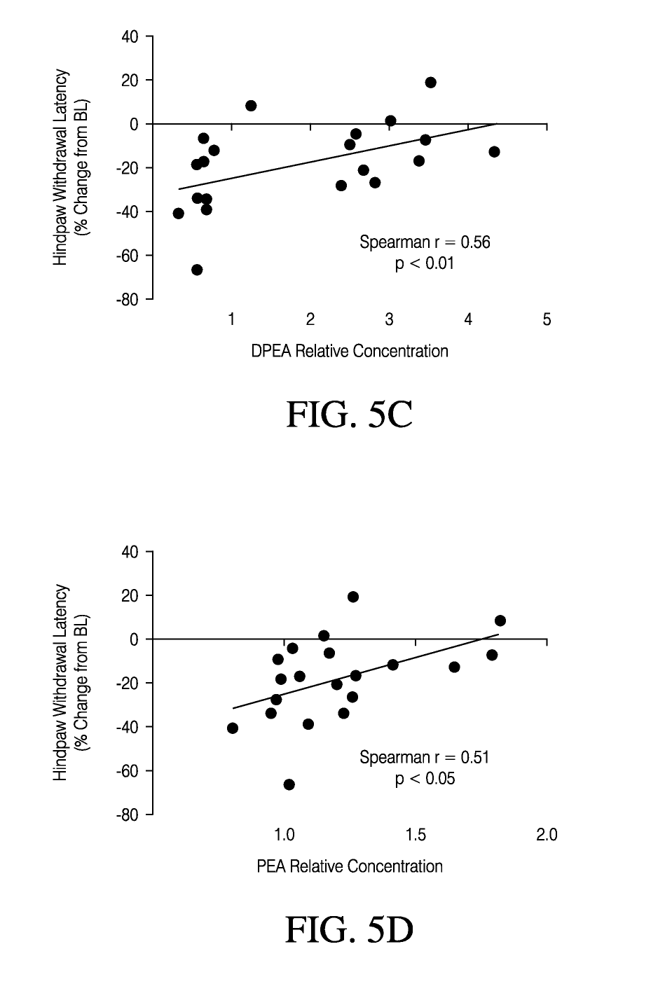

Spearman's correlation analyses were used to explore the linear trends between cord metabolite levels and changes in thermal thresholds. Remarkably, the relative levels of NAEs containing 22 carbons N-acyl chains and the glycerophospho NAEs of O3PUFAs were positively correlated with reduced thermal withdrawal latency changes as shown in FIGS. 5A-D (Spearman r values>0.50; p<0.05).

FIGS. 5A-D depicts metabolic features correlated with pain-like phenotypes. Scatter plot shows the significant relationship between the levels of ethanolamines containing 22-carbon N-acyl chains (FIG. 5A), GP-DHEA (FIG. 5B), GP-DPEA (FIG. 5C), PEA (FIG. 5D) and the hindpaw thermal withdrawal latency change from baseline. For every correlation, the Spearman r was higher than 0.50, with a p<0.05, XY=20 pairs.

A significant positive correlation between the relative levels of palmitoyl ethanolamine (PEA) and non-hyperalgesic responses were found, supporting its anti-inflammatory roles in SCI. Therefore, these diet-derived metabolites may play significant roles in antinociception.

### Functional Metabolomics Implicate the NAEs Biosynthetic Pathways in SCI-Induced CNP

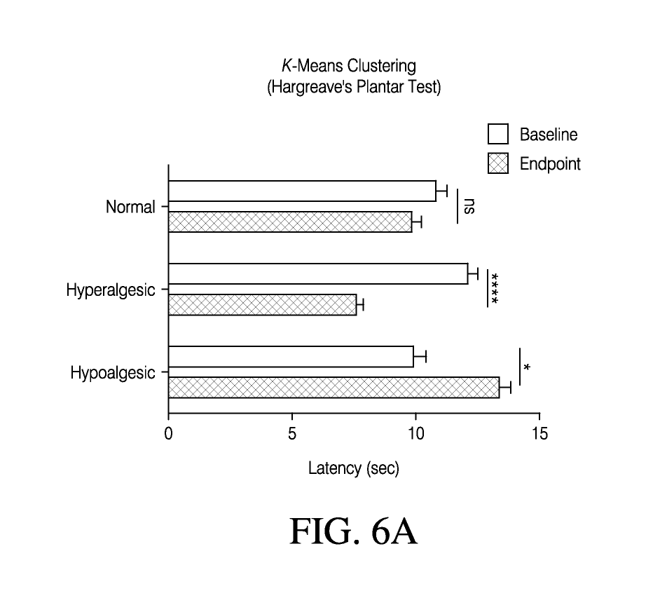

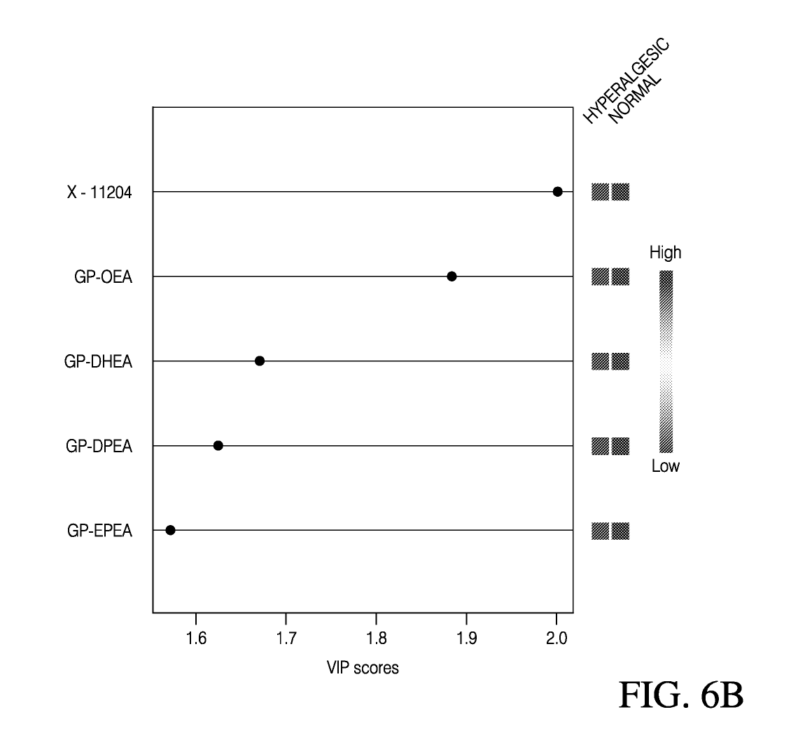

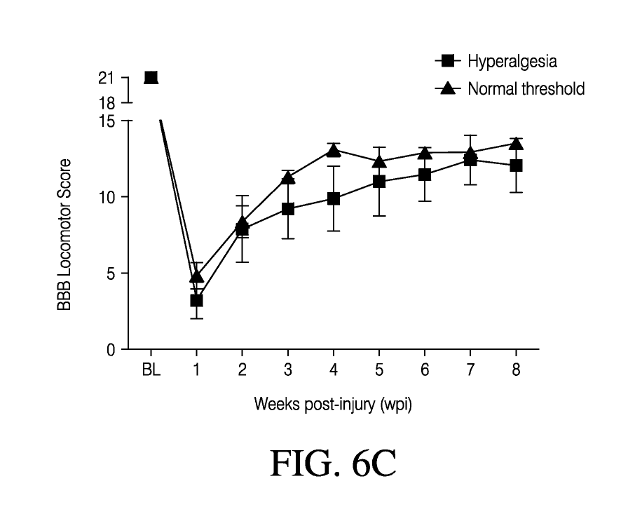

The biochemical basis and etiology underlying CNP remains poorly understood and has limited the development of effective interventions. FIG. 6A shows K-means clustering divided animal based on their nociceptive behavior (Δ latency=latency_{endpoint}−latency_{baseline}). A group of animals exhibited no significant Δ latency changes at endpoint (“normal” or non-hyperalgesic cluster). The clustering algorithm identified two additional groups: hyperalgesic rats (significant A latency decrease at endpoint) and hypoalgesic (increased Δ latency at endpoint). Data is presented as mean±SEM. Data was analyzed by TW-ANOVA followed by Bonferroni's post hoc: ns, not significant; (*)=p<0.05; (****)=p<0.0001. FIG. 6B shows metabolomic analyses using the normal and hyperalgesic clusters confirmed the potential role of the NAE metabolism in pain processing after SCI. In particular, the O3PUFA-derived NAEs were shown to have a significant impact in the metabolic differences observed between thermal pain behaviors. Notably, the animals showing hyperalgesic phenotypes exhibited reduced levels of these metabolites. Abbreviations include: X-11204, unnamed compound which has been tentatively identified as an unsaturated hydroxyl fatty acid with an empirical formula of C₁₃H₂₄O₃; GP, glycerophospho; OEA, oleoyl ethanolamine; DHEA, docosahexanoyl ethanolamine; DPEA, docosapentaenoyl ethanolamine; EPEA, eicosapentaenoyl ethanolamine FIG. 6C depicts that Basso, Beattie and Bresnahan locomotor scores measured in hyperalgesic (n=6) and non-hyperalgesic (n=13) rats showed no significant differences (p>0.05), indicating that the extent of the injury was similar in both groups. This observation proposes underlying neurochemical mechanisms that may be independent of the injury severity and to the amount of spared tissue. Data represents mean±SEM.

To characterize the unique lipidomic changes underlying CNP-like behaviors after contusive SCI as shown in FIG. 6A, the K-means clustering method was used to assign the animals to three groups according to their thermal threshold changes (FIG. 6A). This partitioning method identified a group of animals that showed no significant alterations in their thermal threshold (normal behavior, non-hyperalgesic; p>0.05). Another cluster was shown to exhibit significantly reduced latencies when compared to their baseline values (hyperalgesic behavior; p<0.05). The algorithm also identified animals with increased thermal latencies at 8 wpi when compared to baseline values (hypoalgesic behavior; p<0.05). The mean baseline values did not differ significantly between clustered groups (p>0.05), demonstrating that the different phenotypes developed after SCI.

Notably, the glycerophospho N-acyl ethanolamines (GP-NAEs) derived from the O3PUFA-rich diet were the most relevant metabolites for explaining the differences between non-hyperalgesic and hyperalgesic animals as shown in FIG. 6B. The metabolomics analyses revealed decreased levels of the O3PUFA-derived GP-NAEs in the hyperalgesic animals, suggesting a potential role for these in CNP.

Although additional factors contribute to the development of CNP following SCI, it is well established that the extent of cord damage is a major determinant. To determine the relative contribution of tissue spared to the observed metabolomics differences, we assessed the cord damage between non-CNP and CNP animals. Because the spinal cords were used for metabolomics studies, we could not determine the extent of injury using stereological techniques. However, the Basso, Beattie and Bresnahan (BBB) locomotor score provides a reliable indirect measure of the injury magnitude. We found that the BBB locomotor scores were not significantly different between clustered groups, indicating that the extent of injury (and repair) was similar as shown in FIG. 6C (p>0.05). This observation validates that additional processes such as chronic neuroinflammation and hyperexcitability play major roles in pathological nociception after SCI.

Animals Fed with a Diet Rich in O3PUFAs Exhibit Reduced Levels of P38 MAPK Expression in Dorsal Horn Neurons Following SCI

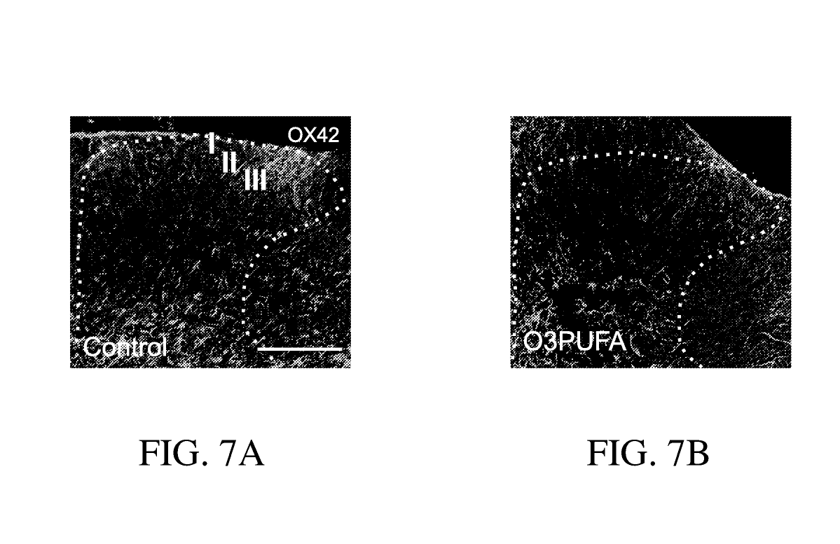

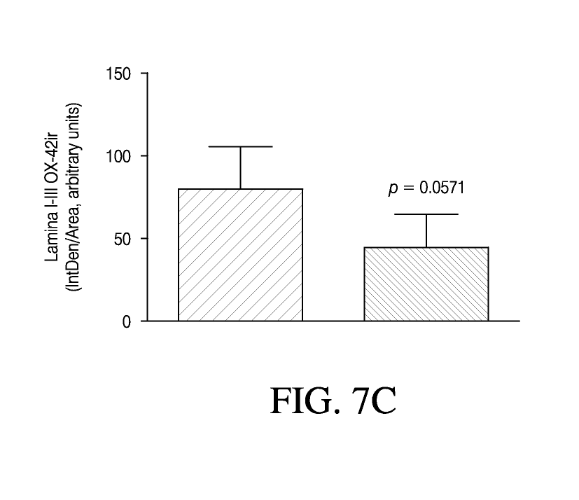

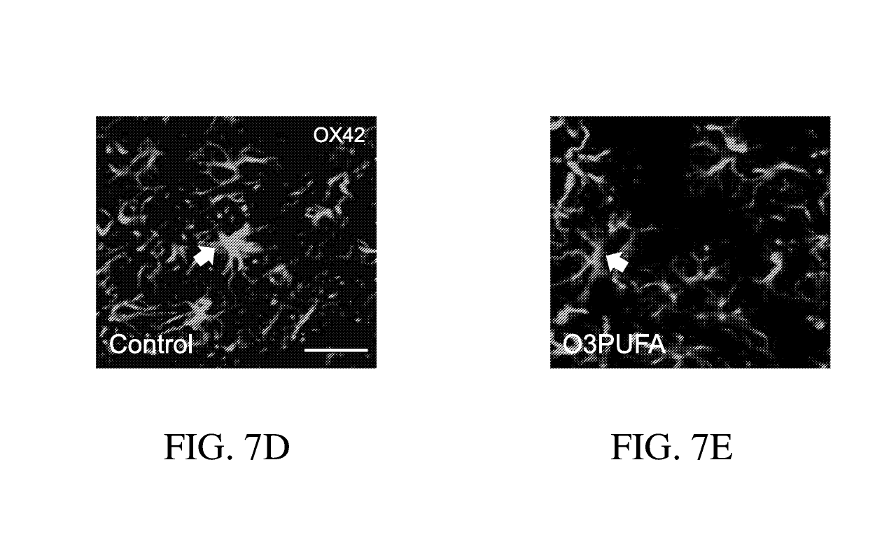

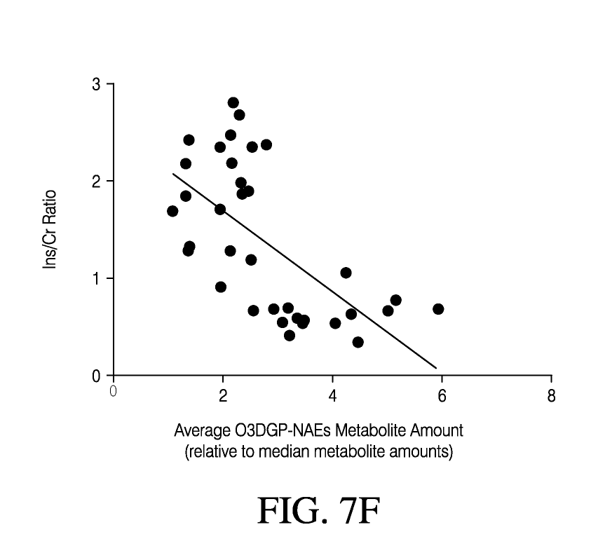

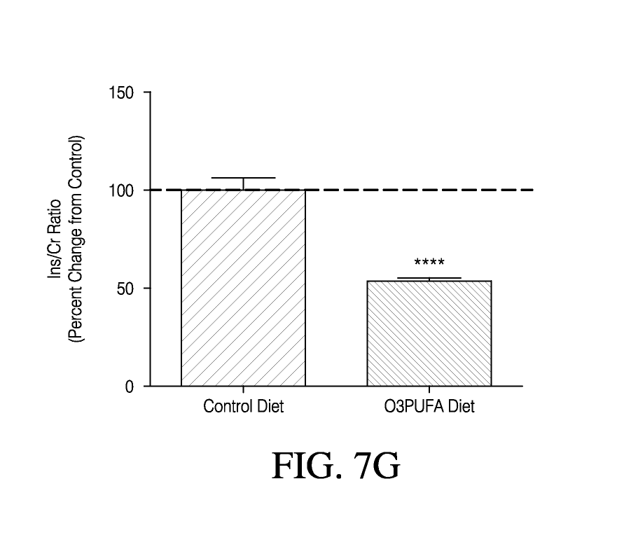

Studies show that inflammatory responses are implicated in the onset and progression of SCI-induced pain. To examine the anti-inflammatory effects of the O3PUFA-enriched diet, the mRNA levels of key cytokines, chemokines, and receptors that have been associated with inflammatory pain (e.g., IL-1β, IL-6, TNF-α, CCL2, CCR2, CX3CL1, and CX3CR1) were determined. The O3PUFA diet did not reduce the mRNA levels of these pro-inflammatory factors in below-level cord segments when compared to control-fed animals at 8 wpi (p>0.05; data not shown). Histological analyses showed no significant differences in the dorsal horn immunoreactivity (IR) to OX42 between treatment groups as shown in FIGS. 7A-C.

FIG. 7A-G demonstrates that dietary O3PUFA did not reduce microglial cell immunoreactivity in superficial dorsal horns following chronic SCI. Representative images from OX42 immunoreacted spinal cord injured caudal sections from animals receiving control (FIG. 7A) or O3PUFA (FIG. 7B) diets. Quantitative analyses showed no significant changes in inflammatory markers immunoreactivity between treatment groups in the dorsal horn laminae I-III at 12 weeks post-injury as shown in FIG. 7C.

These results do not contradict the well-established roles of dietary O3PUFAs but rather suggest that these anti-inflammatory effects may occur during the initial inflammatory trigger in a time- and context-dependent manner. This observation also points to the involvement of additional mechanisms. For instance, although significant differences in the expression of these biomarkers were not observed, qualitative differences were noticeable in cell morphology. In particular, microglia with morphological features typically implicated in activated states in the spinal cords of animals receiving control chow (e.g., hypertrophied cell bodies and thick processes) were found as shown in FIG. 7D. Interestingly, a few animals receiving the dietary O3PUFA intervention exhibited microglial cells with small soma containing thin and radially projecting processes as shown in FIG. 7E, confirming previous observations on the DHA-elicited immunomodulatory effects in microglia.

As shown in FIG. 7D, closer examination revealed that following chronic SCI, spinal microglia displayed hypertrophied cell bodies and thick processes, which are characteristic of their activated state. Interestingly, some animals treated with dietary O3PUFA showed microglial cells with small soma containing thin and radial projecting processes (resting state of microglia) as shown in FIG. 7E. Scale bar=A-B, 200 μm; D-E, 20 μm. The arrows indicate OX42-positive cell somata. Dietary O3PUFA-derived GP-NAEs levels are potentially implicated in anti-inflammatory responses. FIG. 7F is a scatter plot that shows the relationship between the levels of the O3PUFA-derived GP-NAEs (O3DGP-NAEs) and the total inositol-to-creatine levels (Ins/Cr). The Spearman r=−0.68, CI(−0.83 to −0.44), p=0.0001, XY=18 pairs includes sham and injury-operated animals fed with control diet. Dietary O3PUFAs resulted in a significant reduction in the Ins levels (Mann-Whitney U test, **** p<0.0001, n=10) as shown in FIG. 7G. Data represents mean±SEM.

LC/MS and GC/MS-based metabolomics provide a more sensitive and selective method to assess inflammatory biomarkers. Here, we measured the levels of inositol (Ins), a biomarker associated with inflammation in SCI. Inositols have been implicated as osmolytes and clinical metabolic markers of inflammation, SCI-mediated chronic pain, and recently as a marker of SCI progression. Since SCI-induced edema and water disturbances may introduce bias in the quantification of osmolytes, the relative levels of Ins were quantified relative to creatine levels (Ins/Cr ratio). Interestingly, the averaged relative levels of the major omega-3 PUFA-derived GP-NAEs were negatively associated with Ins relative levels (Spearman r=−0.68, p<0.0001) as shown in FIG. 7F. Further, dietary O3PUFAs significantly reduced the cord Ins levels as shown in FIG. 7B (p<0.0001, n=10). Altogether, this data support an anti-inflammatory and antinociceptive role for O3PUFAs in chronic SCI.

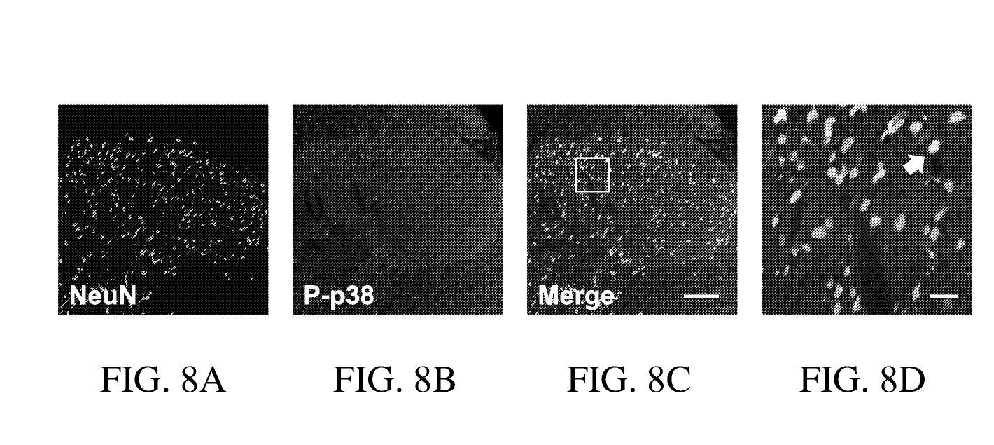

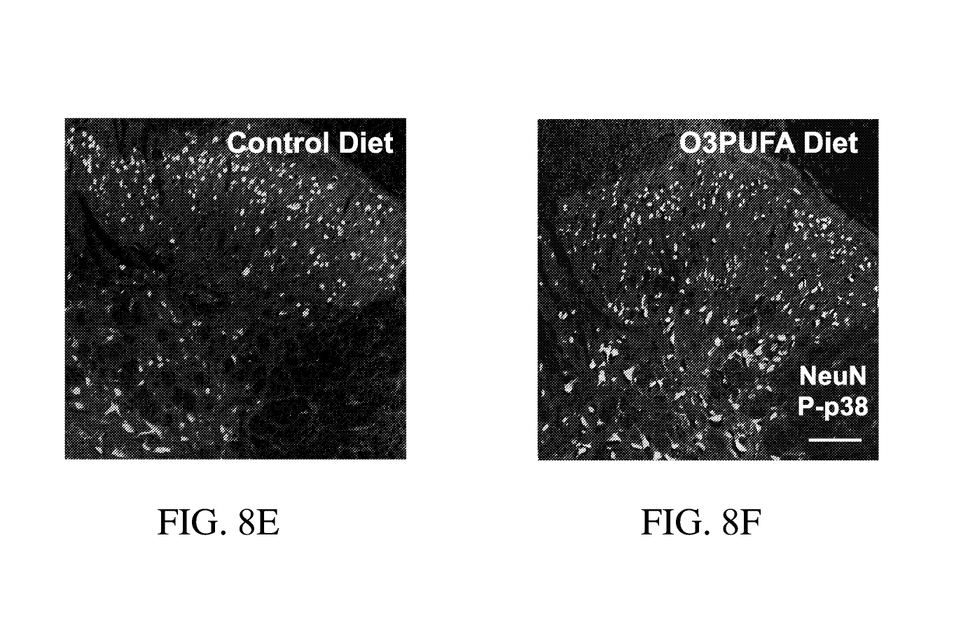

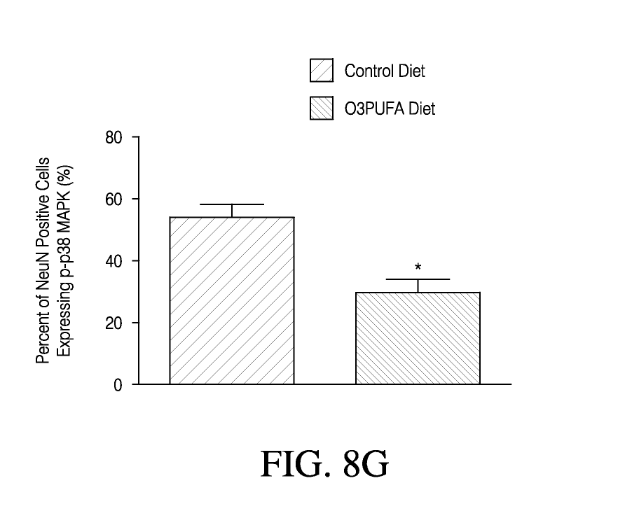

A number of pharmacological studies implicate the spinal p38 mitogen-activated protein kinase, p38 MAPK, as one important underlying mechanism of CNP and neuronal hyperexcitability in SCI Immunohistochemistry was used to examine the expression of this established pain biomarker in rats treated with dietary O3PUFAs relative to animals receiving control diets. The cords of animals receiving O3PUFAs had decreased phosphorylated p38-positive neurons in the superficial dorsal horns relative to controls at 12 wpi as depicted in FIGS. 8A-G (p<0.05; n=at least 4 animals). We found a significant positive correlation between the expression levels of neuronal p38 MAPK and the hyperalgesic behaviors.

FIG. 8A-G shows that preventive dietary O3PUFAs reduce the expression of phosphorylated p38 in below-level dorsal horn neurons. At 12 weeks post-injury, laser confocal microscopic evaluation revealed dorsal NeuN-positive neurons (FIG. 8A) containing the phosphorylated p38 MAPK (FIG. 8B). FIG. 8C shows merged photomicrographs and the FIG. 8D inset show distinctive neuronal subpopulations expressing this inflammatory marker after chronic SCI. Dorsal horn photomicrographs show noticeable differences in the number of p-p38-containing neurons when comparing dietary groups (control=Figure E vs. O3PUFA=Figure F). FIG. 8G shows that manual cell counts confirmed that the dietary O3PUFA intervention significantly reduced the percent of NeuN-positive cells expressing p38 MAPK in below-level dorsal horn superficial laminae (p<0.05; n=at least 4 animals). Scale bars=A-C and E-F, 100 μm; D, 20 μm.

### Dietary O3PUFA Reduces the Sprouting of CGRP-Containing Fibers in Chronic SCI

Calcitonin gene-related peptide (CGRP) has been proposed as a major nociceptive neurotransmitter in SCI. More recent studies support that pain after SCI is due, at least in part, to sprouting of CGRP pathways. The growth-associated protein 43 (GAP43) is highly enriched in growth cones and has been widely used as a marker of sprouting and neuropathic pain. Thus, whether the O3PUFA-enriched diet reduces the sprouting of CGRP-containing primary afferents following chronic SCI was tested.

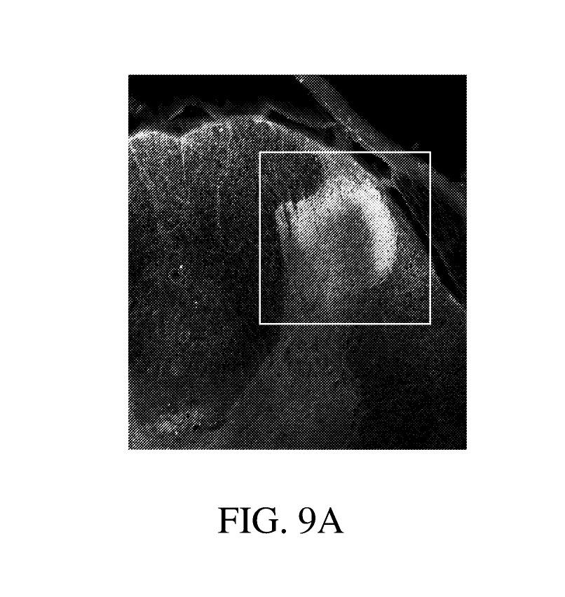

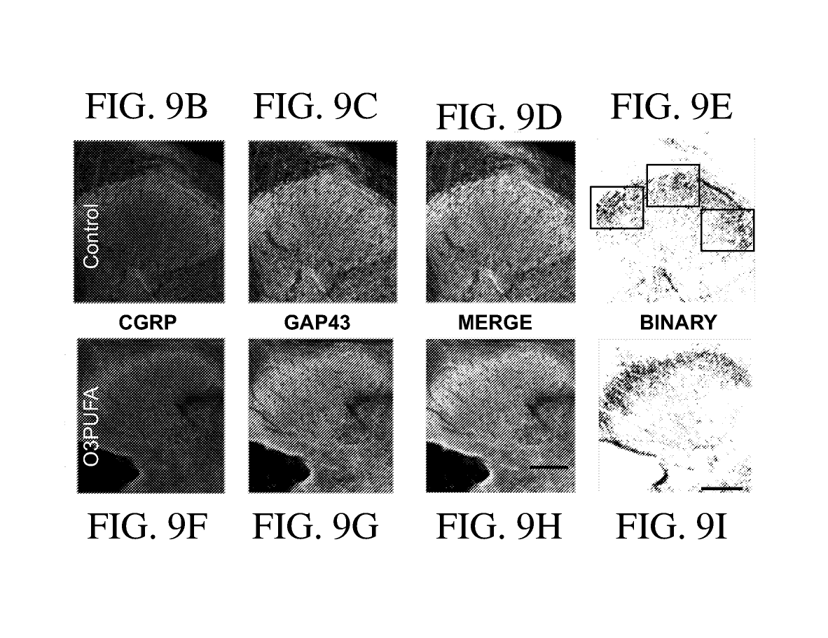

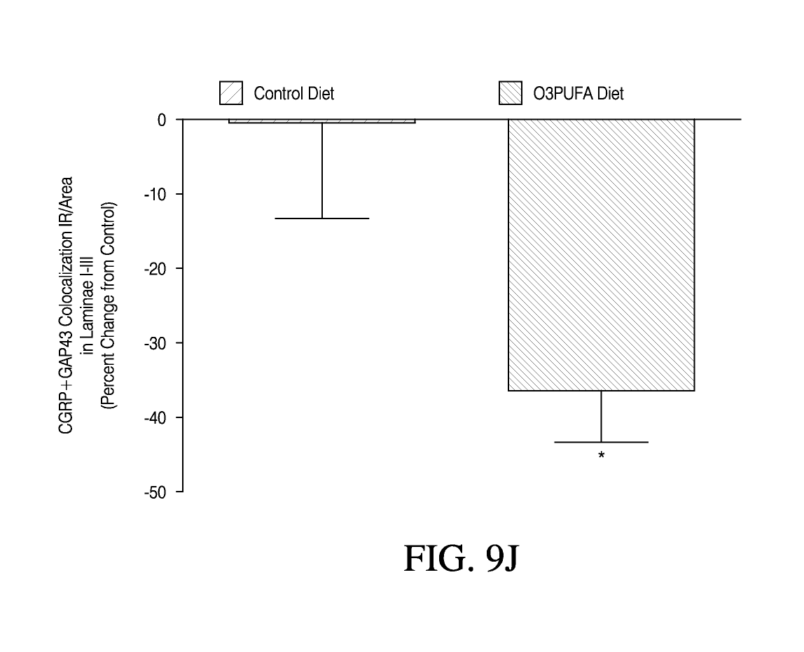

FIG. 9A-J shows that dietary O3PUFA-pretreatment reduces nociceptive fiber sprouting following chronic SCI. Double labeled spinal cord section showing calcitonin gene-related peptide (CGRP) and growth-associated protein 43 (GAP43) immunoreactivity (IR) in spinal cord is shown in FIG. 9A. FIG. 9B and FIG. 9C are confocal photomicrographs showing CGRP (FIG. 9B) and GAP43 (FIG. 9C) immunoreactivity in control chow-fed rat. FIG. 9D depicts merged images to quantify the immunoreactivity of CGRP-containing sprouting afferent fibers. Optical density was most intense in laminae I to III of the dorsal horns. As depicted in FIG. 9E, sections were morphometrically analyzed using stereological methods after thresholding binary images using ImageJ software. FIG. 9F-FIG. 9G are representative image from CGRP (FIG. 9F) and GAP43 (FIG. 9G) immunoreactivity in an animal fed O3PUFA-enriched diets. FIG. 9H and FIG. 9I are merged (FIG. 9H) and binary (FIG. 9I) images that depict CGRP-containing GAP43⁺ fibers in the dorsal horn. Boxes represent areas showing colocalization. Scale bar=100 μm. FIG. 9J shows results from quantification of binary particle counts of dorsal horn superficial laminae Analysis showed that O3PUFA-enriched diet significantly reduced the colocalization of GAP43 and CGRP, suggesting a reduction in nociceptive fiber sprouting. Bars represent means±standard error of the mean; Mann Whitney U test *p<0.05, n=at least 4 rats.

Laser confocal microscopy showed CGRP and GAP43 colocalization in spinal cord sections obtained from regions 3-5 mm caudal to the injury site as shown in FIG. 9A. CGRP (FIGS. 9B,F) and GAP43 (FIGS. 9C,G) labeled photomicrographs from animals receiving control chow (FIGS. 9B-E) or O3PUFA-rich (FIGS. 9F-I) diets were merged (FIGS. 9D,H) and subsequently converted to binary format to facilitate automated analyses (FIGS. 9E,I). Quantitative double labeling immunofluerescence revealed decreased sprouting of CGRP-positive primary afferents at 12 wpi (Mann Whitney U test p<0.05, n=at least 4 animals) as shown in FIG. 9J.

This shows that a preventive diet rich in omega-3 polyunsaturated fatty acids (O3PUFAs) can reduce thermal hyperalgesia in rats experiencing chronic spinal cord injury (SCI). The antihyperalgesic effect is directly correlated with the levels of a series of novel glycerophospho ethanolamines containing O3PUFA acyl chains. The anti-inflammatory effects of the O3PUFA-enriched diet are evident by a significant reduction in levels of inflammatory biomarkers, including cord inositols and the phosphorylated p38 MAPK in dorsal horn neurons.

Chronic neuropathic pain (CNP) is a debilitating co-morbidity associated with SCI and persists even at the later stages of recovery and rehabilitation. This condition often manifest as evoked pain, including hyperalgesia (amplified pain response to noxious stimuli) and/or allodynia (painful response to innocuous stimuli). The intensity and frequency of CNP is particularly influenced by trauma-induced neurochemical and neuroanatomical changes in synaptic circuitry and dorsal horn neuron hyperexcitability. Current approaches to treat CNP include behavioral, pharmacological, and surgical modalities, however, none of these interventions are regarded as highly effective. This could be partly due to patients being treated after considerable and perhaps irreversible changes have developed. There is thus a need to develop preventive approaches to build resilience to damage. Complementary with current strategies, this type of approach may be particularly important in individuals at high risk of traumatic injuries like those actively participating in contact sports, selected surgeries, first responders, and our men and women in the military service. The promise of using preventive approaches to treat CNP is supported by studies showing that central pain can be prevented by pre-administration of opiates, anti-inflammatory molecules, or by prophylactic cell transplantation strategies. Although it may seem unreasonable to use these approaches in individuals undergoing SCI due to potential and unwanted side effects, prophylactic treatment with dietary O3PUFAs offers an excellent profile of clinical safety and may be beneficial in preventing pain and dysesthesias in individuals at risk.

O3PUFAs, such as docosahexaenoic acid (DHA) and eicosapentaenoic acid (EPA), are ubiquitous lipid messengers that regulate crucial neural processes in health and disease. Dietary O3PUFAs exert a stringent control over phospholipid production and are principal determinants of the cord lipid composition following chronic SCI. When the spinal cord is enriched with O3PUFAs before SCI, these lipids mediate robust neuroprotection, recovery, and activate pro-restorative responses. Additionally, the importance of PUFAs in nociception has been shown, supporting the conclusion that dietary lipids are key elements of the nociceptive pathways. Consistent with this idea, the present results show that consumption of a diet rich in O3PUFAs produces significant antihyperalgesic effects in the rat contusion SCI model. In contrast, thoracic contusion to the cord resulted in a significant reduction in below-level thermal withdrawal latencies in animals receiving control chow. Although this latency changes may simply reflect hyperreflexic responses, these animals also exhibited significant sensory deficits in response to pressure after SCI. This paradoxical combination of sensory loss in similar regions where pain is felt discards hyperreflexia as a potential mechanism and further suggests that both neurodegenerative and pro-inflammatory responses may play a role in SCI-mediated neuropathic pain. Notably, comparable open-field locomotor scores were found when animals were grouped based on their thermal withdrawal phenotypes. While neurodegenerative differences between clusters may be subtle or undetectable as measured by the BBB locomotor scores, this finding is evidence supporting additional underlying causes of neuropathic pain.

The neurometabolomic etiology of CNP has been poorly understood and this has limited the development of effective therapeutics. N-Acylated ethanolamines (NAE) and related endocannabinoids (eCBs) are a large class of naturally occurring lipids that exhibit diverse bioactivities, including neuroprotective and antinociceptive responses. Notably, it has been shown that SCI rats exhibit profound alterations in the metabolic pathways associated with these lipids. These lipids are produced on demand from membrane phospholipids and glycerophospho-linked precursors by a series of intracellular enzymatic reactions, followed by immediate signaling and metabolism. The result described herein show that chronic SCI results in a marked deregulation in the metabolic pathways of NAEs and related eCBs. In particular, reduced levels of palmitoyl ethanolamine (PEA) were found in the chronically injured cord, which were correlated with hyperalgesic behaviors in SCI rats. PEA has been implicated in anti-inflammatory responses and functional recovery after SCI and reduces pain-like behaviors in experimental models of neuropathic pain. Notably, dietary prophylaxis with O3PUFAs sustained the levels of PEA after SCI. Further, the O3PUFA-derived NAEs identified in this study are precursors of docosahexaenoyl ethanolamine (DHEA; synaptamide) and eicosapentaenoyl ethanolamine (EPEA), which bind to cannabinoid receptors in rats and may contribute to the beneficial effects mediated by dietary O3PFAs. Although the metabolic pathways involved in NAE biosynthesis remain unclear, our results strongly suggest that NAPE-PLD-independent (N-acyl phosphatidyl ethanolamine phospholipase D) pathways are activated in chronic SCI and represent a promising therapeutic target. Because NAEs can modify the response to nociceptive stimuli and are tightly regulated by diet, O3PUFAs could have important implications for chronic pain management.

The neuroinflammatory milieu after SCI can be linked to changes in sensory electrical activity and pain-related behaviors. For instance, the activation of the spinal p38 mitogen-activated protein kinase, p38 MAPK, as a molecular mechanism underlying neuronal hyperexcitability and pain after SCI has been implicated. Animals consuming dietary O3PUFAs exhibited reduced numbers of dorsal neurons expressing p-p38, indicating a potential molecular link between dietary lipids and pain. This finding is supported by studies demonstrating that both DHA and EPA alone attenuate the activation of p38 in endothelial cells stimulated by TNF-α. DHA also impairs p38 MAPK signaling in microglial cultures. In agreement with these findings on the potential anti-inflammatory and antinociceptive roles of dietary O3PUFAs, a marked reduction in the levels of inositols and CGRP-positive sprouting fibers in the chronically injured cord is shown as described herein. The p38 MAPK has been linked to the regulation of inositol levels in human peripheral blood monocytes and macrophages and to the expression of calcitonin gene-related peptide in rats. The dynamic interplay among these biomarkers of the nociceptive system is shown. These molecular interactions can be evaluated and investingated to determine their potential as useful biomarkers to discriminate pain severity in SCI-related neuropathic pain.

In summary, dietary O3PUFAs prophylaxis attenuates the development of thermal hyperalgesia following SCI, possibly by providing a better bioavailability for anti-inflammatory lipid mediators. Even though recent advances in pain research suggest that combinatorial strategies to both prevent and combat CNP are a feasible goal, identifying targets with the intention of preventing pain is an enormous conceptual challenge that has so far stymied drug discovery. The treatment as described herein supports the use of preventive alternative approaches in individuals at risk of developing CNP and identifies diet as a potential risk factor for poor outcome. This treatment can have remarkable public health implications to reduce the burden of pain, particularly in populations at risk. Because dietary O3PUFAs are safe, well tolerated, and confer robust protection against experimental SCI they should be favored for early pain management in human SCI.

### Neurorestorative Targets of Dietary Long-Chain Omega-3 Fatty Acids in Treatment of Neuropathic Pain

Functional metabolomics studies were conducted to identify and define the dominant metabolic pathways targeted by dietary Long-chain omega-3 polyunsaturated fatty acids (LC-O3PUFAs). Sprague-Dawley rats were fed rodent purified chows containing menhaden fish oil-derived LC-O3PUFAs for 8 weeks before being subjected to sham or spinal cord contusion surgeries. Through untargeted metabolomics, that dietary LC-O3PUFAs regulate important biochemical signatures associated with amino acid metabolism and free radical scavenging in both the injured and sham-operated spinal cord are shown. Of particular significance, the spinal cord metabolome of animals fed with LC-O3PUFAs exhibited reduced glucose levels (−48%) and polar uncharged/hydrophobic amino acids (less than −20%) while showing significant increases in the levels of antioxidant/anti-inflammatory amino acids and peptides metabolites, including β-alanine (+24%), carnosine (+33%), homocarnosine (+27%), kynurenine (+88%), when compared to animals receiving control diets (p<0.05). Further, it was found that dietary LC-O3PUFAs impacted the levels of neurotransmitters and the mitochondrial metabolism, as evidenced by significant increases in the levels of N-acetylglutamate (+43%) and acetyl CoA levels (+27%), respectively. Interestingly, this dietary intervention resulted in a global correction of the pro-oxidant metabolic profile that characterized the SCI-mediated sensorimotor dysfunction. In summary, the significant benefits of metabolic homeostasis and increased antioxidant defenses unlock important neurorestorative pathways of dietary LC-O3PUFAs against SCI.

The initial physical insult sustained in SCI triggers a longer secondary damage that leads to inflammation, demyelination, and apoptosis ultimately leading to dysfunction. This secondary injury phase is characterized by metabolic alterations, glutamate-induced excitotoxicity, and oxidative stress. The levels of these reactive oxygen (ROS) and nitrogen species (NOS) increase considerably when the metabolism is compromised, which can result in irreversible damage to cell membrane lipids, proteins and nucleic acids. ROS scavengers, including catalase, glutathione (GSH), and superoxide dismutase (SOD), are endogenous defense mechanisms that combat oxidative damage. Although there is no current cure for SCI, accumulating clinical and experimental evidence support interventions that target these restorative pathways and hold tremendous promise in ameliorating neurological dysfunction.

Long-chain omega-3 polyunsaturated fatty acids (LC-O3PUFAs) modulate multiple pathways that contribute to secondary damage following SCI. The administration of LC-O3PUFAs restores the cord lipid homeostasis, confers neuroprotection, prevents sensorimotor dysfunction and neuropathic pain, and facilitates locomotor recovery following acute and chronic SCI when administered before the injury. However, there is very limited understanding of the pathways activated by dietary LC-O3PUFAs in the injured central nervous system.

Dietary fatty acids exert potent effects on cellular metabolism through tightly regulated mechanisms at the transcriptional, posttranscriptional, translational, or posttranslational levels. The global non-lipid targets of nutritional LC-O3PUFAs, which offers the advantage of linking dietary LC-O3PUFA-gene interactions to distinctive metabolites and small molecules was investigated. The biologically meaningful metabolic networks that are influenced by dietary LC-O3PUFAs during the acute and chronic injury phases following SCI are identified. In addition, the putative biochemical signatures associated with resiliency against SCI are identified.

### Materials and Methods

### Animals

Female Sprague-Dawley rats were received from Charles River Laboratories (Portage, Mich.) and single housed in environmentally enriched cages on alternating 12 hours light/dark cycles after being acclimated to the new environment for 1 week.

### Experimental Design and Diets

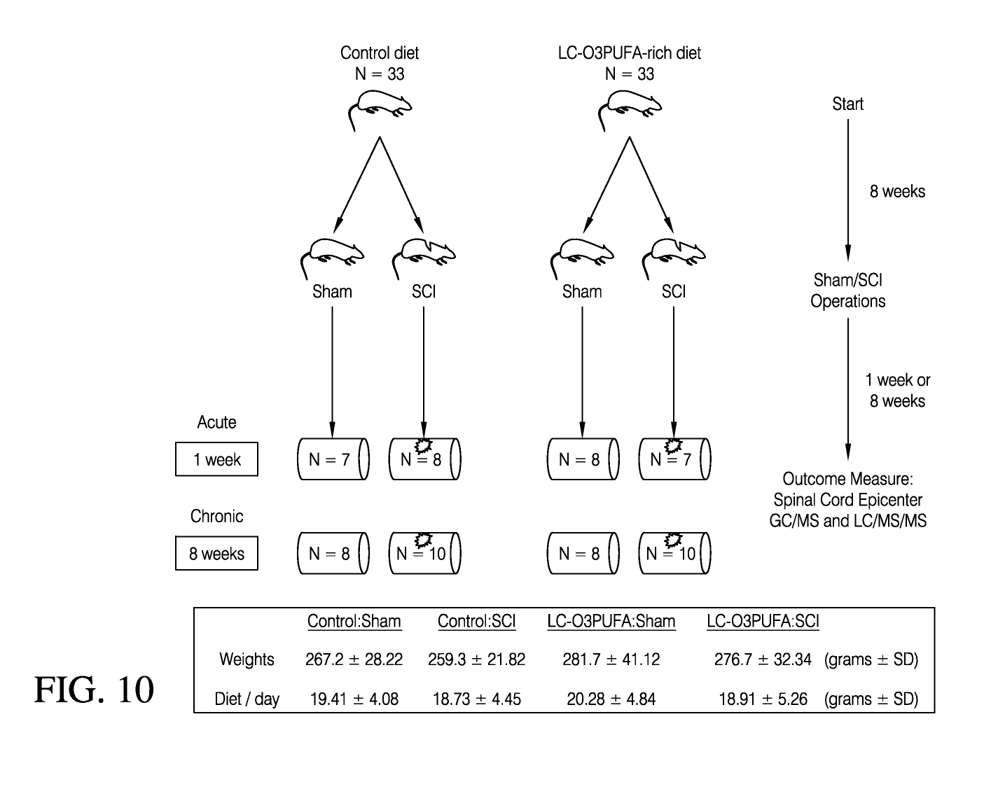

Young adult rats (185-200 g) were fed control or fish oil-enriched diets for 8 weeks, were subjected to sham injury or spinal cord injury, and subsequently allowed to recover for 1 or 8 weeks after trauma. Spinal cord tissue was collected for global metabolic profiling (n=7-10 samples per group). Two independent cohorts were used including: cohort 1: at least seven animals per diet group, allowed to survive until 1 week post-operation; and cohort 2: at least eight animals per diet group, allowed to survive 8 weeks post-operation. Behavioral data from rats in cohort 2 was previously reported. FIG. 10 summarizes the timeline outlining the experimental design. For each group, the average animal weight and daily food consumption is expressed in grams±standard deviation.

FIG. 10 depicts a timeline outlining experimental design and animal groups. Rats were fed control or LC-O3PUFA-enriched chows for 8 weeks before being subjected to sham or SCI operations. Rats were removed for terminal global metabolomics analyses during acute and chronic injury stages. Values for weights and food consumption are average grams (g)±S.D; n=at least 16 rats per group.

Custom AIN-93G-based diets were prepared with modifications to the omega-3 fatty acid source as described previously. Typical analysis of the AIN-93G formulation reveals 7.1% fat, containing cholesterol (0 ppm), linoleic acid (3.58%), linolenic acid (0.55%), arachidonic acid (0%), omega-3 fatty acids (0.55%), total saturated fatty acids (1.05%), total monosaturated fatty acids (1.54%), and poly-unsaturated fatty acids (3.78%). The dietary omega-3 fatty acids were supplied as either soybean oil (control chow) or menhaden fish oil (DHA=12.82 g and EPA=6.91 g per 100 g of fish oil). Because the fish oil-based diet contained 6.23 g of fish oil per 100 g of diet, it was estimated that feeding a 270-g rat with approximately 20 g of diets (or 1.25 g of fish oil per day) should result in a daily intake of approximately 60 mg of DHA and 32 mg of EPA per 100 g of body weight. The total absolute amount of ingested LC-O3PUFAs may vary when additional sources of omega-3 in the AIN-93G diet are considered. Mass-spec analysis further revealed that the level of cholesterol in the menhaden fish oil was 0.582 g/100 g. Cholesterol was added to control diets to match this level. The diets were stored at 4° C. and used fresh. The amount of food ingested was recorded daily during weekdays and averaged during weekends. Table 2 summarizes the composition of the diets.

Table 2 includes a detailed compositional analysis of AIN-93G-based diets. The level of dietary fat was approximately 6% of dry weight supplied as either soybean oil (control chow) or menhaden fish oil (LC-O3PUFA-enriched chow). Gas chromatography coupled with mass spectrometry demonstrated that the level of DHA and eicosapentaenoic acid (EPA) in the menhaden fish oil was 12.82-g and 6.91-g, respectively, per 100 g of diet. The level of cholesterol was 0.582-g/100 g. This amount was added to control diets to make the levels consistent with that of the fish oil diet.

TABLE 2

| | | AIN-93G |
|---|---|---|
| | AIN-93G | Fish oil- |
| Ingredient | Control diet (%) | enriched diet (%) |
| Casein | 20 | 20 |
| L-cystine | 0.3 | 0.3 |
| Corn starch | 39.7 | 39.7 |
| Maltodextrin | 13.2 | 13.2 |
| Sucrose | 10 | 10 |
| Fiber | 5 | 5 |
| Vitamin mix | 1 | 1 |
| Mineral mix | 3.5 | 3.5 |
| Choline bitartrate | 0.25 | 0.25 |
| tbBHQ | 0.0014 | 0.0014 |
| Soybean oil | 7 | 0.77 |
| Fish oil (DHA + EPA + | 0 | 6.23 |
| cholesterol) | | |
| Cholesterol (added to match | 0.0121 | 0 |
| fish oil levels) | | |
| Omega-3 fatty acids in oils | | |
| DHA | <1 g/100 g soybean | 12.82 g/100 g fish oil |
| EPA | <1 g/100 g soybean | 6.91 g/100 g fish oil |
| omega-6:omega-3 | 7.5:1 | 1:3.7 |

### Surgical and Postoperative Procedures

Eight weeks after the dietary intervention, animals were deeply anesthetized with a mixture of ketamine/xylazine (80 mg/kg and 10 mg/kg, respectively). The New York University (NYU) Impactor was used to generate a contusive lesion to the thoracic 10 level of the spinal cord. The spinal cord was subjected to weight drop impact using a 10-g rod released from a height of 12.5-mm. Sham animals received only a laminectomy surgery. The animals' body temperature was maintained at 37° C. during surgery. The muscle layers were then sutured and the skin layers closed. Postoperative care of SCI rats included manual bladder expression at least two times a day until the return of spontaneous urination. Cefazolin (Bristol Myers Squibb, New York, N.Y.; 25 mg/kg, s.q.) and Buprenex® (buprenorphine; Reckett and Colman Pharmaceuticals, Inc. Richmond, Va.; 0.05 mg/kg, s.c.) were also given to all rats for 5 and 3 consecutive days, respectively. Animals were allowed to survive for 1 or 8 weeks post-operation and the spinal cord tissue dissected for metabolomics analysis.

### Metabolomic Profiling

Global metabolic profiling was performed as described herein. Animals were deeply anesthetized and perfused with ice-cold PBS. The spinal cord tissue (75-100 mg) was dissected and put into liquid nitrogen and then stored at −80° C. until use. The tissue samples were homogenized in water and the protein precipitated with methanol containing four standards to report on extraction efficiency. The resulting supernatant was split into equal aliquots for analysis on the three independent platforms: ultrahigh performance liquid chromatography/tandem mass spectrometry (UHPLC/MS/MS2) optimized for basic species, UHPLC/MS/MS2 optimized for acidic species, and gas chromatography/mass spectrometry (GC/MS). The metabolites were identified by comparing the ion features in the experimental samples and to a reference library of chemical standards that includes retention time, molecular weight (m/z), preferred adducts, and in-source fragments as well as associated MS spectra. The biochemical features were curated by visual inspection for quality control using the software developed at Metabolon.

### Metabolomics Analyses

The false discovery rate (FDR) was calculated. The q value describes the false discovery rate and takes into account the multiple comparisons.

The partial least square-discriminant analysis (PLS-DA) was used to identify predictors between groups. This regression method provides information that can predict the class membership (Y) via linear combination of original variables (X). The separation distance permutation was performed to assess the significance of class discriminations between groups. The variable importance in projection (VIP) measures the impact of each metabolite in the model. VIP is a weighted sum of squares of the PLS loadings and takes into account the amount of explained Y-variation in each dimension. Biochemicals with values above 1 are considered important contributors to the group discriminations.

The metabolomic functional analyses were generated through the use of Ingenuity Pathways Analysis (IPA) (www.ingenuity.com). The relative metabolite ratios obtained from metabolomics analyses were converted to fold-change values by the IPA software. The data set was filtered to include metabolites that were associated with biological functions in the Ingenuity Pathways Knowledge Base. The IPA algorithm uses the p value (p<0.05) to determine the probability that each biological function assigned to that data set is due to random chance alone. This value is calculated by considering the number of metabolites in the dataset that participate in that function and the total number of features that are known to be associated with that function in the IPA knowledge database. The level of statistical significance was set at a p value less than 0.05, suggesting non-random associations. We also used established analytical approaches to aid in the interpretation of our large data set. For a biochemical reaction in which (A+B) substrates yield (C+D) products, then low levels of A and/or B with concomitant increases in C and/or D suggest increased metabolism towards formation of products. The inverse scenario was interpreted as accumulation of substrates.

### Statistical Analysis

Statistical analyses were performed using SPSS version 20.0 (IBM: SPSS, Armonk, N.Y.), Prism 5 software v5d (GraphPad Software Inc., San Diego, Calif.), the “R” program (http://cran.r-project.org/), metaboanalyst, and IPA. ANOVA contrasts were used to identify features that differed significantly between groups. Associations were made with the Spearman's rank correlation tests in order to explore relationships between the oxidative profile and the functional phenotype. Data are presented as mean±SD, unless otherwise specified. The differences were considered statistically significant at p<0.05.

### Results

Preventive administration of docosahexaenoic acid (DHA) or consumption of a diet rich in long-chain omega-3 polyunsaturated fatty acids (LC-O3PUFAs) confers potent prophylaxis against multiple SCI co-morbidities and improves functional recovery. However, the mechanisms underlying these beneficial effects remain largely unknown. The impact of LC-O3PUFA dietary supplementation on the spinal cord non-lipid metabolic responses during the acute and chronic phases of SCI recovery are characterized as well as in the sham-operated spinal cord.

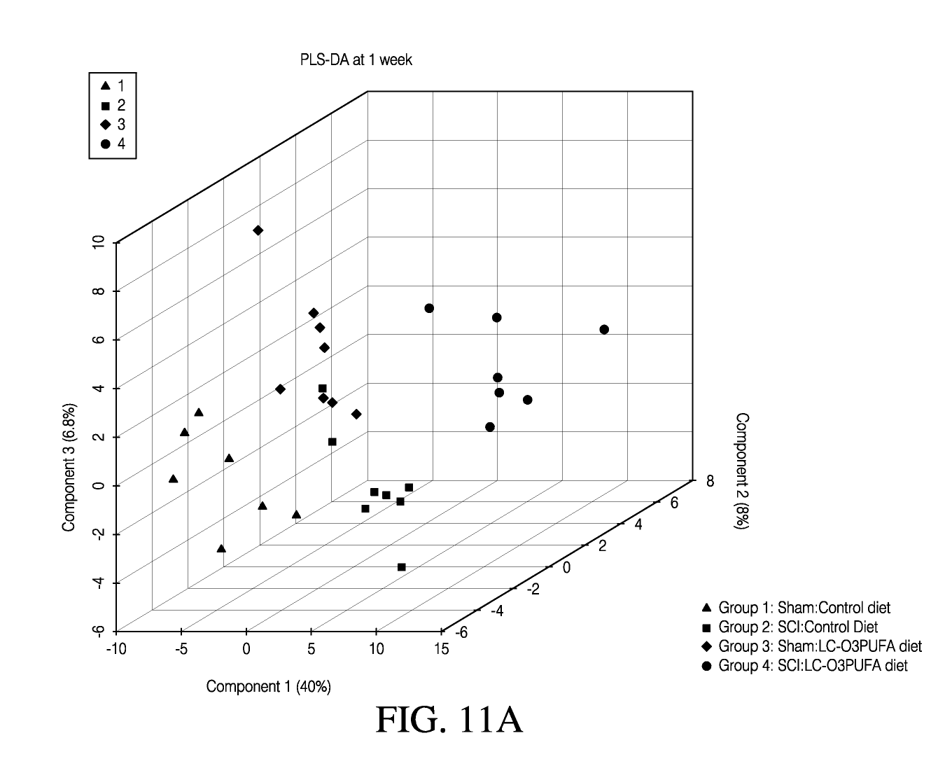

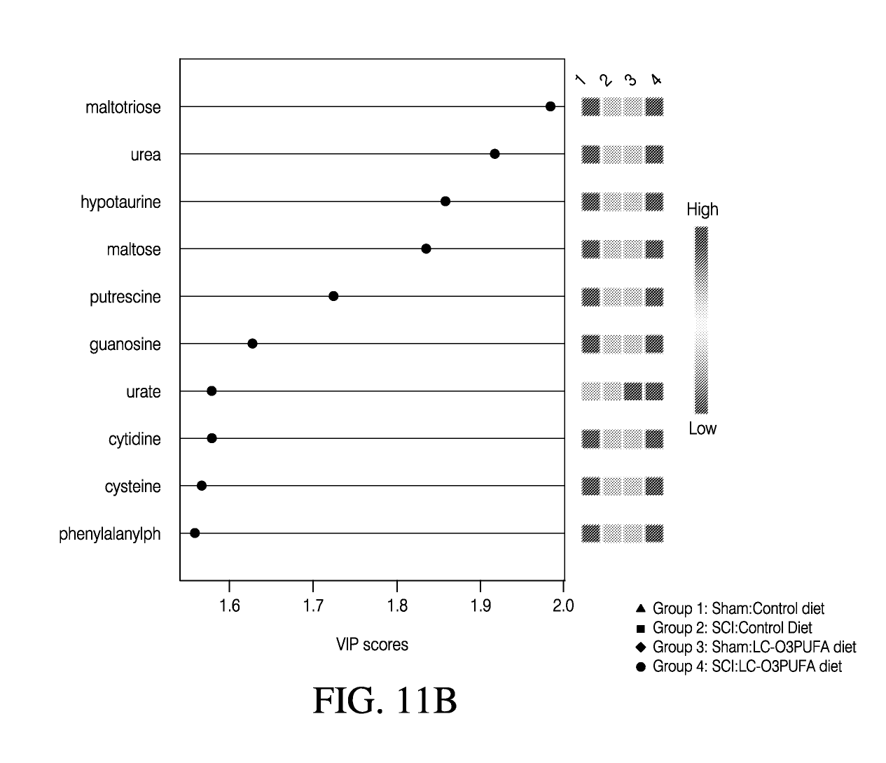

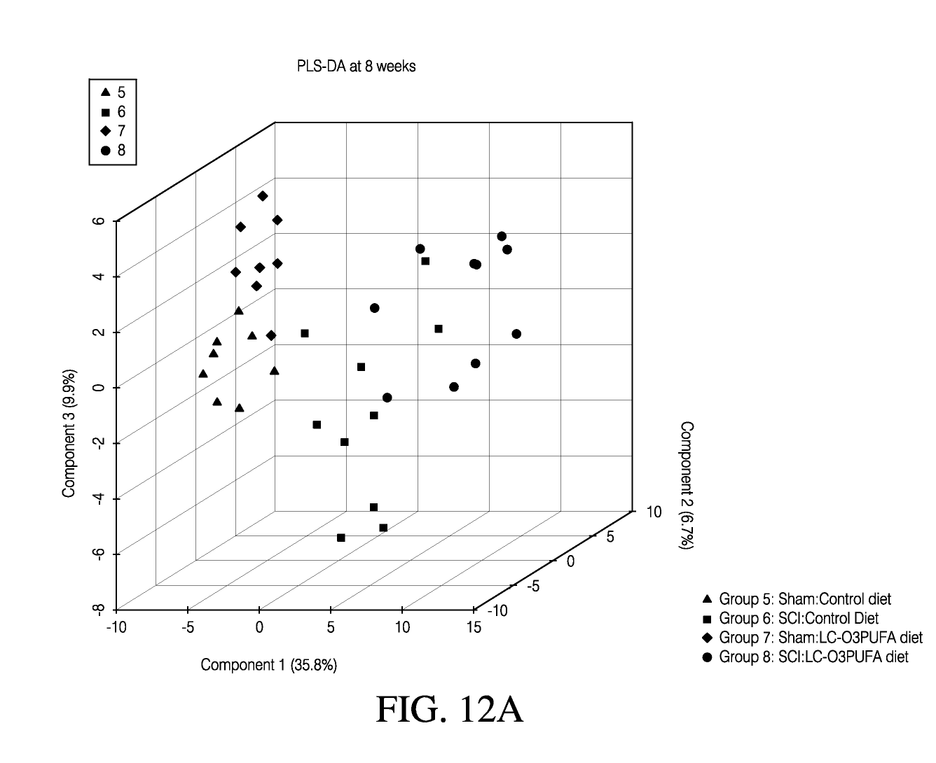

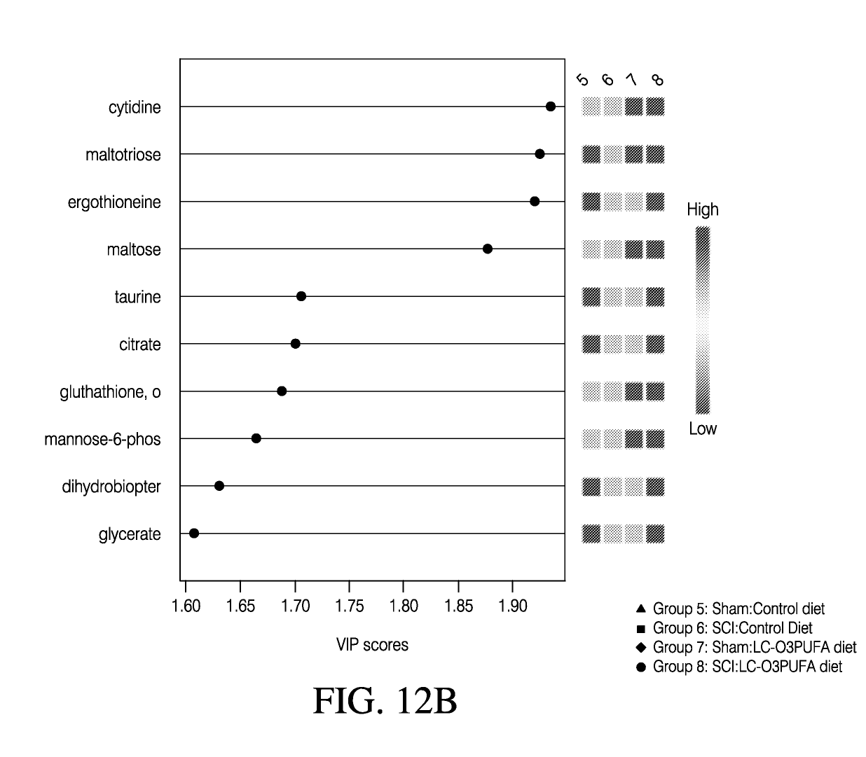

The ability to measure and study dietary LC-O3PUFA's targets and derivatives has been facilitated by the availability of untargeted metabolomics. Because the neurometabolome is tightly regulated, this technique allows for detection of very subtle alterations in biochemical pathways. The PLS-DA score plot was obtained using the variation scores of the first three principal components. In the generated regression models, these components explained more than 50% of the differences between groups at 1 and 8 weeks post-surgery as shown in FIGS. 11A and 12A. Each plot mark corresponds to a rat in the study and the variability in metabolite levels that were detected for that animal. Permutation analyses validated the class discrimination (observed test statistic p<0.05 in both models). PLS analyses revealed that the diet enriched in LC-O3PUFAs had a significant impact in the levels of selective carbohydrates, amino acids, and small peptides with antioxidant capabilities. These small molecules showed the strongest influence to the observed metabolomics differences between groups. This was evidenced by VIP values above 1 as shown in FIGS. 11B and 12B.

FIGS. 11A-11B shows that dietary LC-O3PUFAs significantly modulate the non-lipid spinal cord metabolome during acute injury stages. Partial least square-discriminant analysis (PLS-DA) distinguished subgroups based on operation and dietary intake at 1 week post-operation as shown in FIG. 11A. This model was constructed using scaled intensity peaks of the global non-lipid detected features. Permutation provided statistically significant separations between sub-groups (p<0.05; not shown). The variable influence on projection (VIP) reflects the importance of amino acids and antioxidant peptides in the generated PLS model as shown in FIG. 11B.

FIG. 12A-B shows that dietary LC-O3PUFAs significantly modulate the non-lipid spinal cord metabolome during chronic injury stages. Partial least square-discriminant analysis (PLS-DA) discriminated between groups based on dietary intake at 8 weeks post-operation as shown in FIG. 12A. The model was generated using scaled intensity peaks of the global non-lipid detected features. Permutation provided statistically significant separations between sub-groups (p<0.05; data not shown). The variable influence on projection (VIP) indicates the importance of carbohydrates, amino acids, and anti-oxidant peptides at 8 weeks post-operation as shown in FIG. 12B.

Here, LC-MS/MS data was analyzed using Ingenuity Pathway Analysis software, which as expected revealed that dietary LC-O3PUFAs preferentially target pathways associated with cellular homeostasis and neurological function as shown in FIG. 13. FIG. 13 shows the results of LC-MS/MS data analysis using Ingenuity Pathways Analysis (IPA) software. The data set was filtered for non-lipid small molecules that met the 1.5-fold cut-off criteria. These metabolites were associated with biological functions in the IPA Knowledge Base. The p value was calculated using right-tailed Fischer's exact test and represents the probability that each biological function assigned to that data set is due to random chance alone. p value<0.05 were considered statistically significant.

In particular, the most significant regulated functions in animals fed diets with enriched in LC-O3PUFAs were related to the transport and metabolism of amino acids and neurotransmitter systems and Ca2+-mediated cell signaling, supporting the role of LC-O3PUFAs as crucial regulators of the neuron-glia signaling network. In addition, the dietary LC-O3PUFAs had a robust impact on the metabolism of peptides and amino acids implicated in the regulation of reactive oxygen and nitrogen species. Table 3 summarizes the most significant functions and the number of molecules that were altered by the LC-O3PUFA-rich diet in sham and injured animals. Table 3 shows the major pathways and functions associated with dietary consumption of LC-O3PUFAs in rats.

TABLE 3

| | Diseases or | | Number of |
|---|---|---|---|
| Category | functions annotation | p value | molecules |
| Diet effect in sham rats | | | |
| Cellular function and maintenance | Cellular homeostasis | 1.94E−04 | 17 |
| Molecular transport | Transport of molecule | 8.00E−04 | 16 |
| Small-molecule biochemistry | Synthesis of nitric oxide | 1.19E−09 | 15 |
| Cell signaling | Synthesis of nitric oxide | 1.19E−09 | 15 |
| Free radical scavenging | Metabolism of reactive | 1.68E−06 | 15 |
| | oxygen species | | |
| Amino acid metabolism | Transport amino acids | 2.84E−11 | 12 |
| Molecular transport | Transport amino acids | 2.84E−11 | 12 |
| Small-molecule biochemistry | Transport amino acids | 2.84E−11 | 12 |
| Molecular transport | Quantity of Ca2⁺ | 5.72E−06 | 12 |
| Cell signaling | Quantity of Ca2⁺ | 5.72E−06 | 12 |
| Diet effect in SCI rats | | | |
| Cellular function and maintenance | Cellular homeostasis | 2.93E−05 | 14 |
| Free radical scavenging | Synthesis of reactive | 9.76E−07 | 12 |
| | oxygen species | | |
| Protein synthesis | Metabolism of protein | 1.02E−06 | 12 |
| Molecular transport | Transport of molecule | 6.82E−04 | 12 |
| Small-molecule biochemistry | Synthesis of nitric oxide | 3.62E−08 | 11 |
| Cell signaling | Synthesis of nitric oxide | 3.62E−08 | 11 |
| Cell-to-cell signaling and interaction | Activation of blood cells | 4.15E−06 | 11 |
| Cell death and survival | Cell survival | 1.64E−03 | 11 |
| Cellular growth and proliferation | Growth of bacteria | 7.29E−11 | 10 |
| Cell signaling | quantity od Ca2⁺ | 2.45E−06 | 10 |

### Dietary LC-O3PUFAs Improve Cellular Bioenergetics and Antioxidant Metabolism in Sham Animals

To gain insight into the potential biochemical targets implicated in the neuroprophylactic responses, the neurometabolome of sham-operated animals was investigated. The diet rich in LC-O3PUFAs significantly altered the metabolism of distinctive amino acids and carbohydrates when compared to sham animals receiving control diets (p<0.05). For instance, ANOVA contrasts revealed that the LC-O3PUFA-rich diet increased the spinal cord levels of carnosine (+33%), homocarnosine (+27%), and the major protective precursor, β-alanine (+24%). The levels of 4-guanidinobutanoate (+34%), which is a common byproduct of arginine metabolism and distant precursor of homocarnosine, were increased in the spinal cord of sham-operated rats fed with LC-O3PUFAs when compared to control animals. Recent evidence suggests that the biochemical pathways implicated in the metabolism of these neuroprotective peptides may protect the spinal cord from inflammation and tissue damage after SCI. The diet slightly decreased the levels of the precursor/metabolite amino acid glutamine (−14%) while glutamate levels remained stable and led to significantly higher Glu/Gln ratios (data not shown), suggesting reduced glutamine synthesis or accelerated recycling/catabolism. The dietary intervention regulated the tryptophan metabolism, as evidenced by increased levels of kynurenine (88%) in the spinal cord of animals fed LC-O3PUFA-rich diets when compared to controls. Kynurenines are important modulators of oxidative stress and neurodegeneration.

A significant reduction in key metabolites implicated in the pentose phosphate pathway, including glucose (−48%), glucose-6-phosphate (−24%) and sedoheptulose-7-phosphate (−46%) was demonstrated. Further, the diet increased the levels of acetyl CoA (27%) when compared to the control diet fed animals. Together with the findings showing a reduction in the levels of polar uncharged and hydrophobic side chain amino acid derivatives, including threonine levels (−28% from control), phosphoserine (−33%), betaine (−23%), and 3-hydroxyisobutyrate (−31%), these results suggest an increased protein turnover and/or amino acid shunting to energetic pathways.

The diet rich in LC-O3PUFA had significantly lower levels of ergothioneine (−30%) at 8 weeks post-operation, suggesting differences in the levels of this naturally occurring amino acid between the dietary oils. The major metabolomic changes observed in sham-operated rats consuming LC-O3PUFAs are summarized in Table 4.

Table 4 shows significant metabolites targeted by dietary LC-O3PUFAs in sham rats. Fold changes when compared to sham animals receiving control diets. The additional columns depict the individual effects of diet and SCI in the modulating the levels of each feature.

TABLE 4

| | Fold- | Diet | SCI |
|---|---|---|---|
| Metabolite | change | Effect | Effect |
| One week post-sham operation | | | |
| (LC-O3PUFA diet/control diet) | | | |
| Threonine | 0.72 | ↓ | = |
| Beta alanine | 1.24 | ↑ | = |
| Glutamine | 0.86 | ↓ | = |
| 2-Aminoadipate | 0.61 | ↓ | = |
| Kynurenine | 1.88 | ↑ | = |
| Urea | 1.44 | ↑ | = |
| Carnosine | 1.33 | ↑ | ↓ |
| Homocarnosine | 1.27 | ↑ | ↓ |
| Eight weeks post-sham operation | | | |
| (LC-O3PUFA diet/control diet) | | | |
| Amino acids with polar uncharged | <0.80 | ↓ | = |
| and hydrophobic side chains; | | | |
| serine, threonine and valine, | | | |
| leucine, isoleucine metabolism | | | |
| 4-Guanidinobutanoate | 1.34 | ↑ | = |
| Fructose | 0.71 | ↓ | = |
| Glucose-SP | 0.76 | ↓ | ↑ |
| Glocose | 0.52 | ↓ | ↑ |
| Sedoheptulose-7-phosphate | 0.54 | ↓ | ↑ |
| N1-methyladenosine | 0.74 | ↓ | = |
| Adenosine 2′-monophosphate (Z-AMP) | 0.79 | ↓ | ↑ |
| Acetyl CoA | 1.27 | ↑ | ↓ |
| Ergothioneine | 0.70 | ↓ | = |

Up arrow upregulation;
down arrow downregulation;
equals symbol not significant change

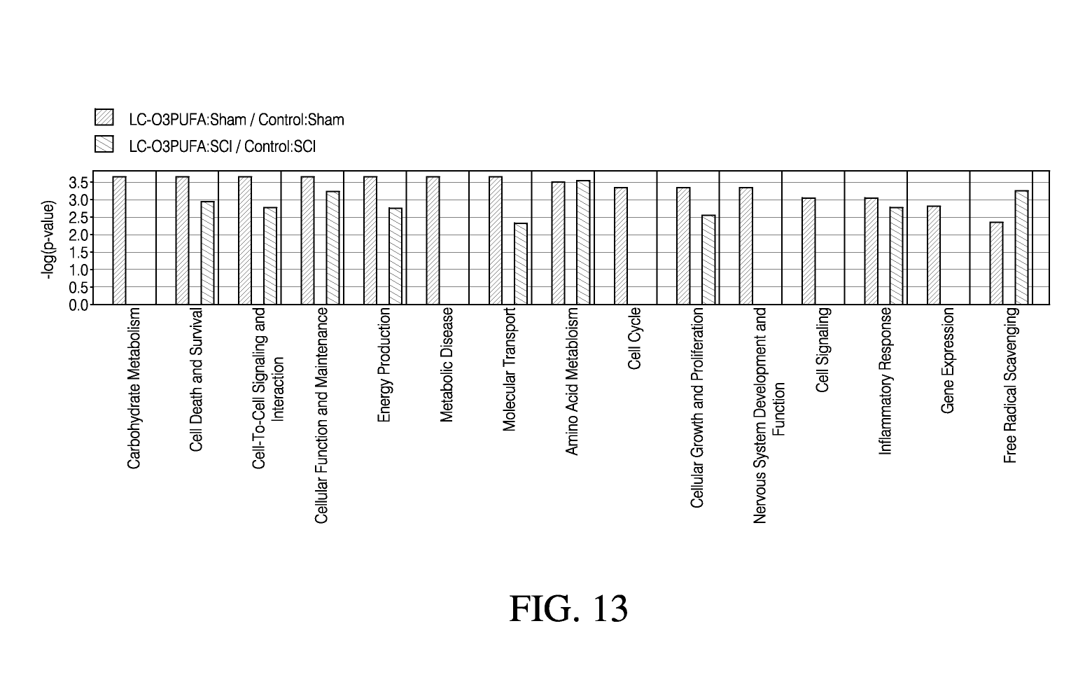

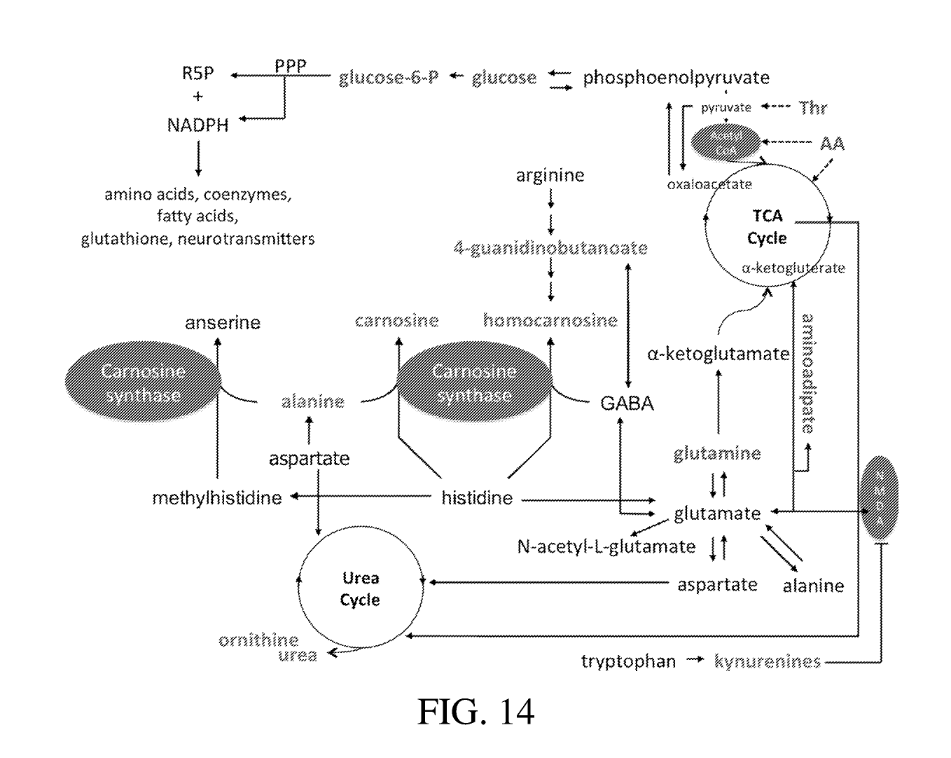

Both IPA and metaboanalyst bioinformatics software were employed to gain insights into the major metabolic targets of dietary LC-O3PUFAs in the sham-operated spinal cord as shown in FIG. 14.

FIG. 14 shows the metabolic pathways targeted by dietary LC-O3PUFA in the sham rat spinal cord. Dietary LC-O3PUFAs target the metabolism of carnosine and homocarnosine, as evidenced by increased levels of alanine, carnosine, homocarnosine, and 4-guanidinobutanoate in the spinal cord of animals fed with LC-O3PUFAs. The diet rich in LC-O3PUFAs also altered the glutamine-glutamate cycling. A distinctive group of amino acid systems were affected by the diet, including threonine and tryptophan. In particular, animals fed with LC-O3PUFAs showed dramatic increases in the levels of kynurenines, which can regulate mitochondrial homeostasis and oxidative stress, inflammation, and glutamate excitotoxicity through NMDA receptor inhibition. Notably, the diet increased the levels of ornithine and urea, while decreasing glucose and glucose-6-P levels, showing selective alterations in the spinal cord cell bioenergetics. This support that LC-O3PUFAs fuel energy production largely by oxidative phosphorylation via the tricarboxylic acid (TCA) cycle and pentose phosphate pathway (PPP) rather than glycolysis, which are essential pathways for the synthesis of necessary macromolecules (i.e., amino acids, neurotransmitters, glutathione, nucleosides and lipids required for assembling new cells). These pathways may represent important mechanisms by which dietary LC-O3PUFAs confer prophylaxis against neurodegeneration and dysfunction in SCI. This reservoir of protective molecules and antioxidant bioavailability is expected to make neurons and glia more resistant against calcium overload, glutamate toxicity, and cell death following SCI. Metabolites such as alanine, carnosine, homocarnosine, 4-guanidinobutanoate, kynurenines, and ornithine urea increased with the dietary intervention. Features such as glucose-6-P, glucose, Thr, AA, aminoadipate, and glutamine decreased with the LC-O3PUFA diet when compared to controls. Putative enzymatic/receptor targets are highlighted in ovals and can include carnosine synthase, NMDA, and Acetyl CoA. Abbreviations include: AA amino acid (polar); GABA gamma-aminobutyrate; NADPH nicotinamide adenine dinucleotide phosphate; NMDA N-methyl-D-aspartate; PPP pentose phosphate pathway; R5P ribose 5-phosphate; Thr threonine.

### Dietary LC-O3PUFAs Target Amino Acid Systems and Complex Carbohydrates at 1-Week Post-SCI

The diet rich in LC-O3PUFAs selectively targeted the metabolism of molecules implicated in oxidative protection and amino acid turnover at 1 week post-injury. In particular, the rats fed with LC-O3PUFAs showed increased levels of cystathionine (+43%), ornithine (+76%), urea (+36%), and hippurate (+219) when compared to injured animals fed control diets.

Another novel finding of this study is that dietary LC-O3PUFAs dramatically upregulated the levels of heme (+292%) in the spinal cord of injured rats at 1 week post-injury (wpi), suggesting increased levels of proteins containing protective heme groups.

Similar to the diet effects in sham-operated rats, we found that the dietary intervention resulted in selective modulation of carbohydrates and the glutamate neurotransmitter system. For instance, dietary LC-O3PUFAs increased the levels of the glucose-containing saccharides maltose (+62%) and maltoriose (+218%). The diet rich in LC-O3PUFAs increased the levels of N-acetylglutamate (+43%) when compared to injured animals fed control diets.

The dietary intervention slightly decreased the levels of methionine (−9%), arginine (−10%), hypoxanthine (−10%), and phosphopantetheine (−16%) when compared to controls at 1-week post-SCI (p<0.05). The metabolomic alterations at 1 wpi are summarized in Table 5.

Table 5 includes important small-molecule targets of LC-O3PUFAs in spinal cord injured rats. Fold changes when compared to injured rats receiving control diets. The additional columns illustrate the individual effects of diet and SCI in the regulating the levels of each biochemical.

TABLE 5

| | Fold- | Diet | SCI |
|---|---|---|---|
| Metabolite | change | Effect | Effect |
| One week post-SCI operation | | | |
| (LC-O3PUFA diet/control diet) | | | |
| N-acetylglutamate | 1.43 | ↑ | ↓ |
| Cystathionine | 1.43 | ↑ | = |
| Methionine and | 0.91, 0.88 | ↑ | ↑ |
| acetylmethionine | | | |
| Arginine | 0.90 | ↓ | ↑ |
| Ornithine | 1.76 | ↑ | ↑ |
| Urea | 1.36 | ↑ | = |
| Maltose and maltotriose | 1.62, 2.18 | ↑ | ↑ |
| Hypoxanthine | 0.90 | ↓ | = |
| Heme | 2.92 | ↑ | = |
| Phosphopantetheine | 0.84 | ↓ | ↓ |
| Hippurate | 2.19 | ↑ | = |
| Eight weeks post-SCI | | | |
| Asparagine | 0.75 | ↓ | = |
| N2-acetyllysine | 1.49 | ↑ | = |
| 2-Aminobutyrate | 0.54 | ↓ | = |
| 5-Methylthioadenosine (MTA) | 1.19 | ↑ | = |
| 4-Guanidinobutanoate | 1.27 | ↑ | = |
| Glutathione reduced (GSH) | 1.42 | ↑ | = |
| Glutathione oxidized (GSSG) | 1.34 | ↑ | ↑ |
| Anserine | 0.50 | ↓ | = |
| Gamma-glutamylglutamine | 1.15 | ↑ | ↓ |
| Maltose | 1.71 | ↑ | ↑ |
| Maltriose | 1.17 | ↑ | = |
| Citrate | 1.30 | ↑ | ↑ |
| Cytidine | 1.20 | ↑ | ↑ |
| Uridine | 1.20 | ↑ | = |
| Methylphosphate | 1.26 | ↑ | = |
| Ergothioneine | 0.52 | ↓ | = |

Up arrow upregulation;
down arrow downregulation;
equals symbol not significant change

### Dietary LC-O3P UFAs Increase Antioxidant Defenses□ and Prevent GSH Depletion in the Chronically Injured Spinal Cord

The animals receiving the LC-O3PUFA-rich diet showed increased glutathione turnover (GSH, +42% and GSSH, +34%) at 8 wpi, suggesting improved antioxidant defenses. The diet slightly increased the levels of γ-glutamylglutamine (+15). This finding provides further evidence of the impact of dietary LC-O3PUFAs in the modulation of the glutamatergic system.

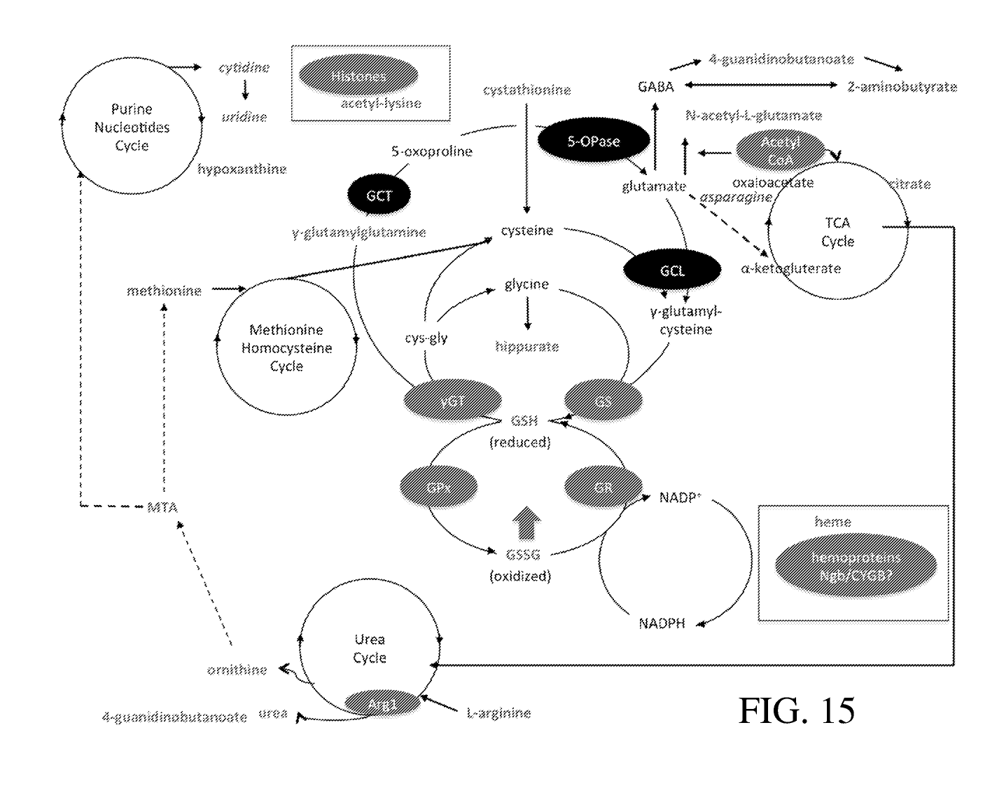

Interestingly, the LC-O3PUFA-rich diet increased the levels of biomarkers associated with cell proliferation and epigenetic mechanism, including cytidine (+20%), uridine (+15%), and N-acetyllysine (+49%) when compared to control fed rats at 8 wpi. Decreased levels of asparagine (−25%), 2-aminobutyrate (−46%), anserine (−50%), and ergothioneine (−48%) were detected in rats consuming LC-O3PUFAs at 8 weeks post-SCI. The metabolic signatures of dietary LC-O3PUFAs at 8 wpi are summarized in Table 5. IPA-assisted metabolic maps were generated to facilitate the understanding of the complex pathways regulated by dietary LC-O3PUFAs as shown in FIG. 15.

FIG. 15 shows the metabolic pathways targeted by dietary LC-O3PUFA in the injured spinal cord. Although we did not characterize the specific sources of ROS in the present study, our metabolomics dataset supports the role of mitochondrial dysfunction as a major source during both acute and chronic injury stages. Interestingly, we identified glutathione (GSH) metabolism as a molecular target of dietary LC-O3PUFAs. For instance, the animals receiving the dietary intervention showed increased spinal cord levels of γ-glutamylglutamine, cystathione, hippurate, GSH, and GSSH, suggesting increased production and/or reduced depletion of antioxidant pools after SCI. Notably, the levels of heme were increased in the spinal cord of rats exposed to the LC-O3PUFA-rich diet, proposing a novel protective mechanism for LC-O3PUFAs. Similar to the findings observed in sham rats, LC-O3PUFAs altered the TCA and Urea cycle. The increased levels of purine nucleotides and acetyllysine suggests a mechanism for which chronic dietary supplementation with LC-O3PUFAs modulates plasticity, growth, and gene expression. Metabolites, such as cytidine, uridine, acetyl-lysine, cystathionine, 4-guanidinobutanoate, N-acetyl-L-glutamate, citrate, γ-glutamylglutamine, hippurate, GSH, GSSSG, heme, MTA, ornithine, 4-guanidinobutanoate, and urea, increased with the dietary intervention. Features, such as L-arginine, methionine, hypoxanthine, asparagine, and 2-aminobutyrate decreased with the LC-O3PUFA diet when compared to controls. Putative enzymatic and protein targets are highlighted in ovals and can include histones, acetyl-CoA, γGT, GS, GPx, GR, hemoproteins Ngb/CYGB, and Arg1. Abbreviations include: 5-OPase 5-oxoprolinase; Arg1 arginase; CYGB cytoglobin; cys-gly cysteine-glycine; GSH glutathione, reduced; GSSH glutathione disulfide, oxidized; GCL γ-glutamylcysteine ligase; GS glutathione synthase; GPx glutathione peroxidase; GR glutathione reductase; GST glutathione-S-transferase; γGT γ-glutamyltransferase; GCT γ-glutamylcylotransferase; MTA 5′-methylthioadenosine; Ngb neuroglobin; TCA tricarboxylic acid.

The complete non-lipid metabolomic profile detected in the spinal cord of the studied groups is represented in Tables 6 and 7. Table 6 includes the complete non-lipid metabolomic profile at 1 week post-operation. Data represents averages of scaled metabolite amount±standard deviation. For each detected metabolite, the raw area counts were resealed to set the median metabolite relative amount equal to 1. The Human Metabolome Database (HMDB) identifier has been provided. The bold indicates averaged median values equal or lower than 1.5-fold from the median metabolite amount of 1, whereas italics indicates averaged median values equal or higher than 1.5-fold from the median metabolite amount.

TABLE 6

| | | | | Control: SCI | | Control: Sham | | O3PUFA: SCI | | O3PUFA: Sham | |
|---|---|---|---|---|---|---|---|---|---|---|---|
| | | | | Average | | Average | | Average | | Average | |
| | | | | Median | | Median | | Median | | Median | |
| | Metabolite | | | Metabolite | | Metabolite | Standard | Metabolite | Standard | Metabolite | Standard |
| | 1 week | HMDB | Subpathway | Levels | Standard Deviation | Levels | Deviation | Levels | Deviation | Levels | Deviation |
| Amino Acids | 2- | HMDB00510 | Lysine | 1.18 | 0.28 | 1.07 | 0.25 | 1.07 | 0.25 | 0.65 | 0.27 |
| | aminoadipate | | metabolism | | | | | | | | |
| | 2- | HMDB00650 | Butanoate | 1.66 | 0.90 | 0.79 | 0.29 | 0.79 | 0.29 | 0.69 | 0.35 |
| | aminobutyrate | | metabolism | | | | | | | | |
| | 2- | HMDB00378 | Valine, leucine | 1.14 | 0.36 | 0.38 | 0.03 | 0.38 | 0.03 | 0.38 | 0.04 |
| | methylbutyroyl | | and isoleucine | | | | | | | | |
| | carnitine (C5) | | metabolism | | | | | | | | |
| | 3-(4- | HMDB00755 | Phenylalanine & | 1.41 | 0.36 | 0.74 | 0.21 | 0.74 | 0.21 | 0.75 | 0.08 |
| | hydroxyphenyl)lactate | | tyrosine | | | | | | | | |
| | (HPLA) | | metabolism | | | | | | | | |
| | 3- | HMDB00336 | Valine, leucine | 1.04 | 0.26 | 0.84 | 0.36 | 0.84 | 0.36 | 0.79 | 0.29 |
| | hydroxyisobutyrate | | and isoleucine | | | | | | | | |
| | | | metabolism | | | | | | | | |
| | 3- | HMDB00272 | Glycine, serine | 0.94 | 0.37 | 1.01 | 0.33 | 1.01 | 0.33 | 1.01 | 0.28 |
| | phosphoserine | | and threonine | | | | | | | | |
| | | | metabolism | | | | | | | | |
| | 4- | HMDB03464 | Guanidino and | 1.22 | 0.18 | 0.80 | 0.26 | 0.80 | 0.26 | 0.82 | 0.20 |
| | guanidinobutanoate | | acetamido | | | | | | | | |
| | | | metabolism | | | | | | | | |
| | 5- | HMDB03355 | Urea cycle; | 1.12 | 0.12 | 0.80 | 0.15 | 0.80 | 0.15 | 0.79 | 0.16 |
| | aminovalerate | | arginine-, | | | | | | | | |
| | | | proline-, | | | | | | | | |
| | | | metabolism | | | | | | | | |
| | 5- | HMDB01173 | Polyamine | 0.88 | 0.16 | 1.08 | 0.17 | 1.08 | 0.17 | 1.11 | 0.14 |
| | methylthioadenosine (MTA) | | metabolism | | | | | | | | |
| | 5-oxoproline | HMDB00267 | Glutathione | 0.93 | 0.07 | 1.08 | 0.09 | 1.08 | 0.09 | 1.09 | 0.13 |
| | | | metabolism | | | | | | | | |
| | alanine | HMDB00161 | Alanine and | 1.17 | 0.16 | 0.78 | 0.10 | 0.78 | 0.10 | 0.82 | 0.10 |
| | | | aspartate | | | | | | | | |
| | | | metabolism | | | | | | | | |
| | arginine | HMDB00517 | Urea cycle; | 1.08 | 0.10 | 0.97 | 0.08 | 0.97 | 0.08 | 0.96 | 0.07 |
| | | | arginine-, | | | | | | | | |
| | | | proline-, | | | | | | | | |
| | | | metabolism | | | | | | | | |
| | asparagine | HMDB00168 | Alanine and | 1.34 | 0.21 | 0.77 | 0.19 | 0.77 | 0.19 | 0.77 | 0.12 |
| | | | aspartate | | | | | | | | |
| | | | metabolism | | | | | | | | |
| | aspartate | HMDB00191 | Alanine and | 0.81 | 0.06 | 1.26 | 0.17 | 1.26 | 0.17 | 1.39 | 0.13 |
| | | | aspartate | | | | | | | | |
| | | | metabolism | | | | | | | | |
| | beta-alanine | HMDB00056 | Alanine and | 0.92 | 0.10 | 0.90 | 0.23 | 0.90 | 0.23 | 1.12 | 0.24 |
| | | | aspartate | | | | | | | | |
| | | | metabolism | | | | | | | | |
| | betaine | HMDB00043 | Glycine, serine | 1.42 | 0.11 | 0.65 | 0.15 | 0.65 | 0.15 | 0.70 | 0.11 |
| | | | and threonine | | | | | | | | |
| | | | metabolism | | | | | | | | |
| | C- | | Tryptophan | 1.41 | 0.59 | 0.93 | 0.23 | 0.93 | 0.23 | 0.94 | 0.18 |
| | glycosyltryptophan* | | metabolism | | | | | | | | |
| | citrulline | HMDB00904 | Urea cycle; | 1.64 | 0.35 | 0.61 | 0.15 | 0.61 | 0.15 | 0.53 | 0.10 |
| | | | arginine-, | | | | | | | | |
| | | | proline-, | | | | | | | | |
| | | | metabolism | | | | | | | | |
| | creatine | HMDB00064 | Creatine | 0.88 | 0.12 | 1.05 | 0.17 | 1.05 | 0.17 | 1.06 | 0.21 |
| | | | metabolism | | | | | | | | |
| | creatinine | HMDB00562 | Creatine | 0.95 | 0.18 | 1.10 | 0.22 | 1.10 | 0.22 | 1.14 | 0.19 |
| | | | metabolism | | | | | | | | |
| | cystathionine | HMDB00099 | Cysteine, | 0.94 | 0.26 | 1.06 | 0.24 | 1.06 | 0.24 | 1.01 | 0.17 |
| | | | methionine, | | | | | | | | |
| | | | SAM, taurine | | | | | | | | |
| | | | metabolism | | | | | | | | |
| | cysteine | HMDB00574 | Cysteine, | 1.04 | 0.15 | 0.90 | 0.15 | 0.90 | 0.15 | 0.95 | 0.09 |
| | | | methionine, | | | | | | | | |
| | | | SAM, taurine | | | | | | | | |
| | | | metabolism | | | | | | | | |
| | cysteine- | HMDB00656 | Glutathione | 1.49 | 0.43 | 0.81 | 0.12 | 0.81 | 0.12 | 0.79 | 0.23 |
| | glutathione | | metabolism | | | | | | | | |
| | disulfide | | | | | | | | | | |
| | cysteine | HMDB00192 | Cysteine, | 0.99 | 0.35 | 0.99 | 0.32 | 0.99 | 0.32 | 0.72 | 0.15 |
| | | | methionine, | | | | | | | | |
| | | | SAM, taurine | | | | | | | | |
| | | | metabolism | | | | | | | | |
| | gamma- | HMDB00112 | Glutamate | 0.72 | 0.09 | 1.29 | 0.15 | 1.29 | 0.15 | 1.40 | 0.23 |
| | aminobutyrate | | metabolism | | | | | | | | |
| | (GABA) | | | | | | | | | | |
| | glutamate | HMDB03339 | Glutamate | 0.86 | 0.07 | 1.10 | 0.07 | 1.10 | 0.07 | 1.14 | 0.11 |
| | | | metabolism | | | | | | | | |
| | glutamine | HMDB00641 | Glutamate | 0.96 | 0.11 | 1.05 | 0.11 | 1.05 | 0.11 | 0.90 | 0.14 |
| | | | metabolism | | | | | | | | |
| | glutathione, | HMDB03337 | Glutathione | 1.80 | 1.87 | 0.95 | 0.27 | 0.95 | 0.27 | 0.97 | 0.35 |
| | oxidized | | metabolism | | | | | | | | |
| | (GSSG) | | | | | | | | | | |
| | glutathione, | HMDB00125 | Glutathione | 1.06 | 0.34 | 1.07 | 0.26 | 1.07 | 0.26 | 1.15 | 0.23 |
| | reduced (GSH) | | metabolism | | | | | | | | |
| | glycine | HMDB00123 | Glycine, serine | 0.95 | 0.12 | 1.08 | 0.14 | 1.08 | 0.14 | 1.15 | 0.13 |
| | | | and threonine | | | | | | | | |
| | | | metabolism | | | | | | | | |
| | histidine | HMDB00177 | Histidine | 1.45 | 0.26 | 0.72 | 0.08 | 0.72 | 0.08 | 0.75 | 0.09 |
| | | | metabolism | | | | | | | | |
| | hydroxyisovaleroylcarnitine (C5) | | Valine, leucine | 1.68 | 0.32 | 0.67 | 0.14 | 0.67 | 0.14 | 0.67 | 0.16 |
| | | | and isoleucine | | | | | | | | |
| | | | metabolism | | | | | | | | |
| | hypotaurine | HMDB00965 | Cysteine, | 1.74 | 0.28 | 0.54 | 0.14 | 0.54 | 0.14 | 0.62 | 0.11 |
| | | | methionine, | | | | | | | | |
| | | | SAM, taurine | | | | | | | | |
| | | | metabolism | | | | | | | | |
| | isoleucine | HMDB00172 | Valine, leucine | 1.43 | 0.15 | 0.82 | 0.07 | 0.82 | 0.07 | 0.82 | 0.07 |
| | | | and isoleucine | | | | | | | | |
| | | | metabolism | | | | | | | | |
| | isovalerylcarnitine | HMDB00698 | Valine, leucine | 1.41 | 0.55 | 0.23 | 0.11 | 0.23 | 0.11 | 0.19 | 0.10 |
| | (C5) | | and isoleucine | | | | | | | | |
| | | | metabolism | | | | | | | | |
| | kynurenine | HMDB00684 | Tryptophan | 2.15 | 3.86 | 2.58 | 5.01 | 2.58 | 5.01 | 4.84 | 4.10 |
| | | | metabolism | | | | | | | | |
| | leucine | HMDB00687 | Valine, leucine | 1.43 | 0.10 | 0.79 | 0.05 | 0.79 | 0.05 | 0.80 | 0.07 |
| | | | and isoleucine | | | | | | | | |
| | | | metabolism | | | | | | | | |
| | lysine | HMDB00182 | Lysine | 1.22 | 0.31 | 0.89 | 0.11 | 0.89 | 0.11 | 0.93 | 0.05 |
| | | | metabolism | | | | | | | | |
| | methionine | HMDB00696 | Cysteine, | 1.28 | 0.11 | 0.86 | 0.06 | 0.86 | 0.06 | 0.88 | 0.06 |
| | | | methionine, | | | | | | | | |
| | | | SAM, taurine | | | | | | | | |
| | | | metabolism | | | | | | | | |
| | N-acetyl- | HMDB01067 | Glutamate | 0.71 | 0.05 | 1.18 | 0.06 | 1.18 | 0.06 | 1.14 | 0.05 |
| | aspartyl- | | metabolism | | | | | | | | |
| | glutamate (NAAG) | | | | | | | | | | |
| | N- | HMDB00765 | Alanine and | 1.25 | 0.15 | 0.88 | 0.10 | 0.86 | 0.10 | 0.95 | 0.05 |
| | acetylalanine | | aspartate | | | | | | | | |
| | | | metabolism | | | | | | | | |
| | N- | HMDB00812 | Alanine and | 0.49 | 0.16 | 1.46 | 0.16 | 1.46 | 0.16 | 1.31 | 0.23 |
| | acetylaspartate | | aspartate | | | | | | | | |
| | (NAA) | | metabolism | | | | | | | | |
| | N- | HMDB01138 | Glutamate | 0.61 | 0.20 | 1.23 | 0.35 | 1.23 | 0.35 | 1.34 | 0.28 |
| | acetylglutamate | | metabolism | | | | | | | | |
| | N- | HMDB11745 | Cysteine, | 1.58 | 0.16 | 0.62 | 0.04 | 0.62 | 0.04 | 0.67 | 0.09 |
| | acetylmethionine | | methionine, | | | | | | | | |
| | | | SAM, taurine | | | | | | | | |
| | | | metabolism | | | | | | | | |
| | N- | | Glycine, serine | 1.27 | 0.29 | 0.78 | 0.10 | 0.78 | 0.10 | 0.77 | 0.11 |
| | acetylthreonine | | and threonine | | | | | | | | |
| | | | metabolism | | | | | | | | |
| | N2- | HMDB00446 | Lysine | 1.04 | 0.28 | 1.18 | 0.22 | 1.18 | 0.22 | 1.08 | 0.24 |
| | acetyllysine | | metabolism | | | | | | | | |
| | N6- | HMDB00206 | Lysine | 1.12 | 0.18 | 0.91 | 0.24 | 0.91 | 0.24 | 0.93 | 0.13 |
| | acetyllysine | | metabolism | | | | | | | | |
| | ornithine | HMDB03374 | Urea cycle; | 1.82 | 0.65 | 0.84 | 0.11 | 0.84 | 0.11 | 0.85 | 0.08 |
| | | | arginine-, | | | | | | | | |
| | | | proline-, | | | | | | | | |
| | | | metabolism | | | | | | | | |
| | phenylalanine | HMDB00159 | phenylalanine & | 1.44 | 0.15 | 0.83 | 0.09 | 0.83 | 0.09 | 0.83 | 0.08 |
| | | | tyrosine | | | | | | | | |
| | | | metabolism | | | | | | | | |
| | pipecolate | HMDB00070 | Lysine | 0.99 | 0.29 | 0.72 | 0.36 | 0.72 | 0.36 | 0.66 | 0.28 |
| | | | metabolism | | | | | | | | |
| | proline | HMDB00162 | Urea cycle; | 1.27 | 0.12 | 0.88 | 0.08 | 0.88 | 0.08 | 0.83 | 0.06 |
| | | | arginine-, | | | | | | | | |
| | | | proline-, | | | | | | | | |
| | | | metabolism | | | | | | | | |
| | putrescine | HMDB01414 | Polyamine | 1.42 | 0.24 | 0.42 | 0.05 | 0.42 | 0.05 | 0.52 | 0.17 |
| | | | metabolism | | | | | | | | |
| | S- | HMDB00939 | Cysteine, | 0.84 | 0.12 | 1.14 | 0.12 | 1.14 | 0.12 | 1.10 | 0.10 |
| | adenosylhomocysteine (SAH) | | methionine, | | | | | | | | |
| | | | SAM, taurine | | | | | | | | |
| | | | metabolism | | | | | | | | |
| | sarcosine (N- | HMDB00271 | Glycine, serine | 0.97 | 0.13 | 1.06 | 0.15 | 1.06 | 0.15 | 1.01 | 0.23 |
| | Methylglycine) | | and threonine | | | | | | | | |
| | | | metabolism | | | | | | | | |
| | serine | HMDB00187 | Glycine, serine | 1.21 | 0.19 | 0.77 | 0.13 | 0.77 | 0.13 | 0.80 | 0.10 |
| | | | and threonine | | | | | | | | |
| | | | metabolism | | | | | | | | |
| | spermidine | HMDB01257 | Polyamine | 1.18 | 0.74 | 0.95 | 0.11 | 0.95 | 0.11 | 1.14 | 0.30 |
| | | | metabolism | | | | | | | | |
| | taurine | HMDB00251 | Cysteine, | 1.70 | 0.51 | 0.72 | 0.22 | 0.72 | 0.22 | 0.78 | 0.18 |
| | | | methionine, | | | | | | | | |
| | | | SAM, taurine | | | | | | | | |
| | | | metabolism | | | | | | | | |
| | threonine | HMDB00167 | Glycine, serine | 1.11 | 0.13 | 0.98 | 0.21 | 0.98 | 0.21 | 0.71 | 0.16 |
| | | | and threonine | | | | | | | | |
| | | | metabolism | | | | | | | | |
| | hydroxyproline | HMDB00725 | Urea cycle; | 1.48 | 0.67 | 0.64 | 0.23 | 0.64 | 0.23 | 0.48 | 0.15 |
| | | | arginine-, | | | | | | | | |
| | | | proline-, | | | | | | | | |
| | | | metabolism | | | | | | | | |
| | tryptophan | HMDB00929 | Tryptophan | 1.23 | 0.10 | 0.82 | 0.11 | 0.82 | 0.11 | 0.83 | 0.13 |
| | | | metabolism | | | | | | | | |
| | tyrosine | HMDB00158 | Phenylalanine & | 1.37 | 0.13 | 0.75 | 0.08 | 0.75 | 0.08 | 0.77 | 0.07 |
| | | | tyrosine | | | | | | | | |
| | | | metabolism | | | | | | | | |
| | urea | HMDB00294 | Urea cycle; | 0.85 | 0.28 | 0.75 | 0.21 | 0.75 | 0.21 | 1.07 | 0.26 |
| | | | arginine-, | | | | | | | | |
| | | | proline-, | | | | | | | | |
| | | | metabolism | | | | | | | | |
| | valine | HMDB00883 | Valine, leucine | 1.42 | 0.11 | 0.84 | 0.06 | 0.84 | 0.06 | 0.85 | 0.06 |
| | | | and isoleucine | | | | | | | | |
| | | | metabolism | | | | | | | | |
| Carbohydrates | dihydroxyacetone | HMDB01882 | Glycolysis, | 1.18 | 0.41 | 0.84 | 0.51 | 0.84 | 0.51 | 1.22 | 0.59 |
| | | | gluconeogenesis, | | | | | | | | |
| | | | pyruvate | | | | | | | | |
| | | | metabolism | | | | | | | | |
| | 1,5- | HMDB02712 | Glycolysis, | 1.24 | 0.23 | 0.74 | 0.22 | 0.74 | 0.22 | 0.81 | 0.29 |
| | anhydroglucitol | | gluconeogenesis, | | | | | | | | |
| | (1,5-AG) | | pyruvate | | | | | | | | |
| | | | metabolism | | | | | | | | |
| | 3- | HMDB00807 | Glycolysis, | 0.99 | 0.20 | 1.03 | 0.14 | 1.03 | 0.14 | 0.93 | 0.16 |
| | phosphoglycerate | | gluconeogenesis, | | | | | | | | |
| | | | pyruvate | | | | | | | | |
| | | | metabolism | | | | | | | | |
| | arabinose | HMDB00646 | Nucleotide | 0.98 | 0.15 | 0.98 | 0.14 | 0.98 | 0.14 | 1.08 | 0.18 |
| | | | sugars, pentose | | | | | | | | |
| | | | metabolism | | | | | | | | |
| | arabitol | HMDB01851 | Nucleotide | 1.05 | 0.52 | 1.21 | 0.36 | 1.21 | 0.35 | 0.99 | 0.29 |
| | | | sugars, pentose | | | | | | | | |
| | | | metabolism | | | | | | | | |
| | dihydroxyacetone | HMDB01473 | Glycolysis, | 0.92 | 0.22 | 0.94 | 0.19 | 0.94 | 0.19 | 1.00 | 0.22 |
| | phosphate | | gluconeogenesis, | | | | | | | | |
| | (DHAP) | | pyruvate | | | | | | | | |
| | | | metabolism | | | | | | | | |
| | erythronate* | HMDB00613 | Aminosugars | 1.26 | 0.29 | 1.02 | 0.14 | 1.02 | 0.14 | 0.80 | 0.11 |
| | | | metabolism | | | | | | | | |
| | fructose | HMDB00660 | Fructose, | 1.24 | 0.40 | 0.79 | 0.13 | 0.79 | 0.13 | 0.91 | 0.34 |
| | | | mannose, | | | | | | | | |
| | | | galactose, | | | | | | | | |
| | | | starch, and | | | | | | | | |
| | | | sucrose | | | | | | | | |
| | | | metabolism | | | | | | | | |
| | fructose 6- | HMDB00124 | Glycolysis, | 1.62 | 0.47 | 0.80 | 0.10 | 0.80 | 0.10 | 0.82 | 0.26 |
| | phosphate | | gluconeogenesis, | | | | | | | | |
| | | | pyruvate | | | | | | | | |
| | | | metabolism | | | | | | | | |
| | glucose | HMDB00122 | Glycolysis, | 2.26 | 1.26 | 0.22 | 0.06 | 0.22 | 0.06 | 0.38 | 0.36 |
| | | | gluconeogenesis, | | | | | | | | |
| | | | pyruvate | | | | | | | | |
| | | | metabolism | | | | | | | | |
| | glucose 6- | HMDB01401 | Glycolysis, | 1.54 | 0.47 | 0.68 | 0.10 | 0.68 | 0.10 | 0.72 | 0.24 |
| | phosphate | | gluconeogenesis, | | | | | | | | |
| | | | pyruvate | | | | | | | | |
| | | | metabolism | | | | | | | | |
| | glycerate | HMDB00139 | Glycolysis, | 1.28 | 0.31 | 0.95 | 0.32 | 0.95 | 0.32 | 0.93 | 0.20 |
| | | | gluconeogenesis, | | | | | | | | |
| | | | pyruvate | | | | | | | | |
| | | | metabolism | | | | | | | | |
| | Isobar: | | Glycolysis, | 1.00 | 0.39 | 0.86 | 0.26 | 0.86 | 0.25 | 1.00 | 0.32 |
| | hexose | | gluconeogenesis, | | | | | | | | |
| | diphosphates | | pyruvate | | | | | | | | |
| | | | metabolism | | | | | | | | |
| | Isobar: | | Nucleotide | 1.05 | 0.24 | 0.94 | 0.09 | 0.94 | 0.09 | 0.96 | 0.18 |
| | ribulose 5- | | sugars, pentose | | | | | | | | |
| | phosphate, | | metabolism | | | | | | | | |
| | xylulose 5- | | | | | | | | | | |
| | phosphate | | | | | | | | | | |
| | lactate | HMDB00190 | Glycolysis, | 1.01 | 0.12 | 0.99 | 0.11 | 0.99 | 0.11 | 1.04 | 0.13 |
| | | | gluconeogenesis, | | | | | | | | |
| | | | pyruvate | | | | | | | | |
| | | | metabolism | | | | | | | | |
| | maltose | HMDB00163 | Fructose, | 1.53 | 0.72 | 0.44 | 0.07 | 0.44 | 0.07 | 0.47 | 0.08 |
| | | | mannose, | | | | | | | | |
| | | | galactose, | | | | | | | | |
| | | | starch, and | | | | | | | | |
| | | | sucrose | | | | | | | | |
| | | | metabolism | | | | | | | | |
| | maltotriose | HMDB01262 | Fructose, | 0.84 | 0.55 | 0.26 | 0.01 | 0.26 | 0.01 | 0.28 | 0.04 |
| | | | mannose, | | | | | | | | |
| | | | galactose, | | | | | | | | |
| | | | starch, and | | | | | | | | |
| | | | sucrose | | | | | | | | |
| | | | metabolism | | | | | | | | |
| | mannitol | HMDB00765 | Fructose, | 0.84 | 0.50 | 0.62 | 0.22 | 0.62 | 0.22 | 0.80 | 0.38 |
| | | | mannose, | | | | | | | | |
| | | | galactose, | | | | | | | | |
| | | | starch, and | | | | | | | | |
| | | | sucrose | | | | | | | | |
| | | | metabolism | | | | | | | | |
| | mannose | HMDB00169 | Fructose, | 1.65 | 0.45 | 0.57 | 0.14 | 0.57 | 0.14 | 0.61 | 0.16 |
| | | | mannose, | | | | | | | | |
| | | | galactose, | | | | | | | | |
| | | | starch, and | | | | | | | | |
| | | | sucrose | | | | | | | | |
| | | | metabolism | | | | | | | | |
| | mannose 6- | HMDB01078 | Fructose, | 1.61 | 0.47 | 0.72 | 0.14 | 0.72 | 0.14 | 0.74 | 0.26 |
| | phosphate | | mannose, | | | | | | | | |
| | | | galactose, | | | | | | | | |
| | | | starch, and | | | | | | | | |
| | | | sucrose | | | | | | | | |
| | | | metabolism | | | | | | | | |
| | N- | HMDB00230 | Aminosugars | 1.03 | 0.11 | 1.04 | 0.20 | 1.04 | 0.20 | 0.87 | 0.18 |
| | acetylneuraminate | | metabolism | | | | | | | | |
| | ribitol | HMDB00508 | Nucleotide | 1.02 | 0.37 | 1.05 | 0.33 | 1.05 | 0.33 | 0.96 | 0.24 |
| | | | sugars, pentose | | | | | | | | |
| | | | metabolism | | | | | | | | |
| | ribose | HMDB00283 | Nucleotide | 0.99 | 0.17 | 1.16 | 0.31 | 1.16 | 0.31 | 1.45 | 0.60 |
| | | | sugars, pentose | | | | | | | | |
| | | | metabolism | | | | | | | | |
| | ribulose | HMDB00621 | Nucleotide | 1.11 | 0.37 | 0.76 | 0.31 | 0.76 | 0.31 | 0.81 | 0.33 |
| | | | sugars, pentose | | | | | | | | |
| | | | metabolism | | | | | | | | |
| | sedoheptulose- | HMDB01068 | Nucleotide | 1.37 | 0.55 | 0.61 | 0.36 | 0.61 | 0.36 | 0.94 | 0.41 |
| | 7- | | sugars, pentose | | | | | | | | |
| | phosphate | | metabolism | | | | | | | | |
| | sorbitol | HMDB00247 | Fructose, | 1.13 | 0.40 | 0.86 | 0.25 | 0.86 | 0.25 | 0.93 | 0.27 |
| | | | mannose, | | | | | | | | |
| | | | galactose, | | | | | | | | |
| | | | starch, and | | | | | | | | |
| | | | sucrose | | | | | | | | |
| | | | metabolism | | | | | | | | |
| | sucrose | HMDB00258 | Fructose, | 0.76 | 0.15 | 0.72 | 0.03 | 0.72 | 0.03 | 1.02 | 0.82 |
| | | | mannose, | | | | | | | | |
| | | | galactose, | | | | | | | | |
| | | | starch, and | | | | | | | | |
| | | | sucrose | | | | | | | | |
| | | | metabolism | | | | | | | | |
| Cofactors and vitamins | 3- | HMDB01373 | Pantothenate | 0.79 | 0.34 | 1.20 | 0.41 | 1.20 | 0.41 | 1.52 | 0.66 |
| | dephosphocoenzyme A | | and CoA | | | | | | | | |
| | | | metabolism | | | | | | | | |
| | alpha- | HMDB01893 | Tocopherol | 1.23 | 0.15 | 0.91 | 0.08 | 0.91 | 0.08 | 0.93 | 0.24 |
| | tocopherol | | metabolism | | | | | | | | |
| | ascorbate | HMDB00044 | Ascorbate and | 1.23 | 0.23 | 0.77 | 0.14 | 0.77 | 0.14 | 0.85 | 0.15 |
| | (Vitamin C) | | aldarate | | | | | | | | |
| | | | metabolism | | | | | | | | |
| | CoA | HMDB01423 | Pantothenate | 0.76 | 0.32 | 1.58 | 0.77 | 1.58 | 0.77 | 1.74 | 0.87 |
| | | | and CoA | | | | | | | | |
| | | | metabolism | | | | | | | | |
| | dehydroascorbate | HMDB01264 | Ascorbate and | 1.03 | 0.48 | 0.92 | 0.22 | 0.92 | 0.22 | 0.90 | 0.35 |
| | | | aldarate | | | | | | | | |
| | | | metabolism | | | | | | | | |
| | dihydrobiopterin | HMDB00038 | Tetrahydrobiopterin | 1.11 | 0.28 | 0.71 | 0.15 | 0.71 | 0.15 | 0.62 | 0.00 |
| | | | metabolism | | | | | | | | |
| | FAD | HMDB01248 | Riboflavin | 1.00 | 0.10 | 0.95 | 0.08 | 0.95 | 0.08 | 1.02 | 0.08 |
| | | | metabolism | | | | | | | | |
| | heme | HMDB03178 | Hemoglobin | 0.71 | 0.12 | 0.68 | 0.05 | 0.68 | 0.05 | 0.80 | 0.27 |
| | | | and porphyrin | | | | | | | | |
| | | | metabolism | | | | | | | | |
| | nicotinamide | HMDB01406 | Nicotinate and | 0.88 | 0.07 | 1.06 | 0.07 | 1.06 | 0.07 | 1.09 | 0.08 |
| | | | nicotinamide | | | | | | | | |
| | | | metabolism | | | | | | | | |
| | NAD+ | HMDB00902 | Nicotinate and | 0.88 | 0.13 | 1.07 | 0.21 | 1.07 | 0.21 | 1.07 | 0.17 |
| | | | nicotinamide | | | | | | | | |
| | | | metabolism | | | | | | | | |
| | pantothenate | HMDB00210 | Pantothenate | 1.00 | 0.16 | 1.00 | 0.16 | 1.00 | 0.16 | 1.08 | 0.20 |
| | (Vitamin B5) | | and CoA | | | | | | | | |
| | | | metabolism | | | | | | | | |
| | phosphopantetheine | HMDB01416 | Pantothenate | 0.91 | 0.10 | 1.12 | 0.13 | 1.12 | 0.13 | 1.18 | 0.14 |
| | | | and CoA | | | | | | | | |
| | | | metabolism | | | | | | | | |
| | pyridoxal | HMDB01545 | Pyridoxal | 1.02 | 0.17 | 0.97 | 0.31 | 0.97 | 0.31 | 1.06 | 0.29 |
| | | | metabolism | | | | | | | | |
| | riboflavin | HMDB00244 | Riboflavin | 1.11 | 0.12 | 0.84 | 0.12 | 0.84 | 0.12 | 0.92 | 0.16 |
| | (Vitamin B2) | | metabolism | | | | | | | | |
| Energy | cis-aconitate | HMDB00072 | Krebs cycle | 1.49 | 0.18 | 0.61 | 0.09 | 0.61 | 0.09 | 0.55 | 0.10 |
| | citrate | HMDB00094 | Krebs cycle | 1.66 | 0.38 | 0.65 | 0.09 | 0.65 | 0.09 | 0.69 | 0.10 |
| | fumarate | HMDB00134 | Krebs cycle | 1.01 | 0.22 | 1.04 | 0.17 | 1.04 | 0.17 | 1.15 | 0.23 |
| | malate | HMDB00156 | Krebs cycle | 1.10 | 0.16 | 0.96 | 0.07 | 0.96 | 0.07 | 1.01 | 0.14 |
| | phosphate | HMDB01429 | Oxidative | 0.97 | 0.04 | 1.02 | 0.03 | 1.02 | 0.03 | 1.07 | 0.05 |
| | | | phosphorylation | | | | | | | | |
| | pyrophosphate | HMDB00250 | Oxidative | 0.85 | 0.50 | 0.75 | 0.41 | 0.75 | 0.41 | 1.03 | 0.66 |
| | (PPI) | | phosphorylation | | | | | | | | |
| | succinylcarnitine | | Krebs cycle | 1.20 | 0.30 | 0.76 | 0.19 | 0.76 | 0.19 | 0.92 | 0.13 |
| | (C4) | | | | | | | | | | |
| Nucleotides | 2′- | HMDB00014 | Pyrimidine | 1.45 | 0.20 | 0.64 | 0.09 | 0.64 | 0.09 | 0.67 | 0.13 |
| | deoxycytidine | | metabolism, | | | | | | | | |
| | | | cytidine | | | | | | | | |
| | | | containing | | | | | | | | |
| | 2′- | HMDB00071 | Purine | 1.15 | 0.44 | 0.46 | 0.00 | 0.46 | 0.00 | 0.46 | 0.00 |
| | deoxyinosine | | metabolism, | | | | | | | | |
| | | | (hypo)xanthine/inosine | | | | | | | | |
| | | | containing | | | | | | | | |
| | 5,6- | HMDB00076 | Pyrimidine | 1.04 | 0.39 | 1.08 | 0.18 | 1.08 | 0.18 | 0.95 | 0.23 |
| | dihydrouracil | | metabolism, | | | | | | | | |
| | | | uracil containing | | | | | | | | |
| | adenine | HMDB00034 | Purine | 0.78 | 0.15 | 1.29 | 0.15 | 1.29 | 0.15 | 1.23 | 0.22 |
| | | | metabolism, | | | | | | | | |
| | | | adenine | | | | | | | | |
| | | | containing | | | | | | | | |
| | adenosine | HMDB00050 | Purine | 0.33 | 0.16 | 1.56 | 0.43 | 1.56 | 0.43 | 1.75 | 0.39 |
| | | | metabolism, | | | | | | | | |
| | | | adenine | | | | | | | | |
| | | | containing | | | | | | | | |
| | 2′-AMP | HMDB11617 | Purine | 1.13 | 0.48 | 0.85 | 0.22 | 0.85 | 0.22 | 0.82 | 0.28 |
| | | | metabolism, | | | | | | | | |
| | | | adenine | | | | | | | | |
| | | | containing | | | | | | | | |
| | 3′-AMP | HMDB03540 | Purine | 1.18 | 0.14 | 0.76 | 0.16 | 0.76 | 0.16 | 0.75 | 0.18 |
| | | | metabolism, | | | | | | | | |
| | | | adenine | | | | | | | | |
| | | | containing | | | | | | | | |
| | AMP | HMDB00045 | Purine | 0.80 | 0.15 | 1.17 | 0.36 | 1.17 | 0.36 | 1.34 | 0.23 |
| | | | metabolism, | | | | | | | | |
| | | | adenine | | | | | | | | |
| | | | containing | | | | | | | | |
| | allantoin | HMDB00462 | Purine | 1.16 | 0.58 | 0.87 | 0.35 | 0.87 | 0.35 | 1.00 | 0.56 |
| | | | metabolism, | | | | | | | | |
| | | | urate | | | | | | | | |
| | | | metabolism | | | | | | | | |
| | cytidine | HMDB00089 | Pyrimidine | 1.09 | 0.07 | 0.94 | 0.04 | 0.94 | 0.04 | 0.96 | 0.06 |
| | | | metabolism, | | | | | | | | |
| | | | cytidine | | | | | | | | |
| | | | containing | | | | | | | | |
| | 3′-CMP | | Pyrimidine | 0.92 | 0.15 | 0.98 | 0.15 | 0.98 | 0.15 | 1.09 | 0.18 |
| | | | metabolism, | | | | | | | | |
| | | | cytidine | | | | | | | | |
| | | | containing | | | | | | | | |
| | guanosine | HMDB00133 | Purine | 0.54 | 0.09 | 1.33 | 0.10 | 1.33 | 0.10 | 1.25 | 0.18 |
| | | | metabolism, | | | | | | | | |
| | | | guanine | | | | | | | | |
| | | | containing | | | | | | | | |
| | hypoxanthine | HMDB00157 | Purine | 1.02 | 0.07 | 1.00 | 0.09 | 1.00 | 0.09 | 1.02 | 0.10 |
| | | | metabolism, | | | | | | | | |
| | | | (hypo)xanthine/inosine | | | | | | | | |
| | | | containing | | | | | | | | |
| | inosine | HMDB00195 | Purine | 0.79 | 0.07 | 1.05 | 0.06 | 1.05 | 0.06 | 1.09 | 0.04 |
| | | | metabolism, | | | | | | | | |
| | | | (hypo)xanthine/inosine | | | | | | | | |
| | | | containing | | | | | | | | |
| | methylphosphate | | Purine and | 0.94 | 0.14 | 1.07 | 0.11 | 1.07 | 0.11 | 1.09 | 0.13 |
| | | | pyrimidine | | | | | | | | |
| | | | metabolism | | | | | | | | |
| | 1- | HMDB03331 | Purine | 1.21 | 0.31 | 0.86 | 0.18 | 0.86 | 0.18 | 0.90 | 0.35 |
| | methyladinosine | | metabolism, | | | | | | | | |
| | | | adenine | | | | | | | | |
| | | | containing | | | | | | | | |
| | pseudouridine | HMDB00767 | Pyrimidine | 1.36 | 0.31 | 0.76 | 0.19 | 0.76 | 0.19 | 0.81 | 0.08 |
| | | | metabolism, | | | | | | | | |
| | | | uracil containing | | | | | | | | |
| | uracil | HMDB00300 | Pyrimidine | 1.30 | 0.17 | 0.76 | 0.14 | 0.76 | 0.14 | 0.78 | 0.12 |
| | | | metabolism, | | | | | | | | |
| | | | uracil containing | | | | | | | | |
| | urate | HMDB00289 | Purine | 1.64 | 0.37 | 0.54 | 0.24 | 0.54 | 0.24 | 0.48 | 0.12 |
| | | | metabolism, | | | | | | | | |
| | | | urate | | | | | | | | |
| | | | metabolism | | | | | | | | |
| | uridine | HMDB00296 | Pyrimidine | 0.77 | 0.10 | 1.17 | 0.12 | 1.17 | 0.12 | 1.10 | 0.18 |
| | | | metabolism, | | | | | | | | |
| | | | uracil containing | | | | | | | | |
| | xanthine | HMDB00292 | Purine | 1.12 | 0.16 | 0.94 | 0.09 | 0.94 | 0.09 | 0.94 | 0.09 |
| | | | metabolism, | | | | | | | | |
| | | | (hypo)xanthine/inosine | | | | | | | | |
| | | | containing | | | | | | | | |
| | xanthosine | HMDB00298 | Purine | 1.26 | 0.25 | 0.78 | 0.14 | 0.78 | 0.14 | 0.72 | 0.15 |
| | | | metabolism, | | | | | | | | |
| | | | (hypo)xanthine/inosine | | | | | | | | |
| | | | containing | | | | | | | | |
| Peptides | anserine | HMDB00194 | Dipeptide | 0.70 | 0.31 | 1.61 | 1.53 | 1.61 | 1.53 | 2.15 | 1.47 |
| | | | derivative | | | | | | | | |
| | carnosine | HMDB00033 | Dipeptide | 0.58 | 0.04 | 0.99 | 0.31 | 0.99 | 0.31 | 1.31 | 0.23 |
| | | | derivative | | | | | | | | |
| | gamma- | | gamma- | 1.37 | 0.23 | 0.80 | 0.15 | 0.80 | 0.15 | 0.80 | 0.22 |
| | glutamylalanine | | glutamyl | | | | | | | | |
| | gamma- | | gamma- | 0.85 | 0.11 | 1.15 | 0.15 | 1.15 | 0.15 | 1.19 | 0.19 |
| | glutamylglutamate | | glutamyl | | | | | | | | |
| | gamma- | HMDB11738 | gamma- | 1.02 | 0.21 | 0.92 | 0.10 | 0.92 | 0.10 | 0.94 | 0.14 |
| | glutamylglutamine | | glutamyl | | | | | | | | |
| | gamma- | HMDB11667 | gamma- | 0.71 | 0.21 | 1.11 | 0.25 | 1.11 | 0.25 | 1.31 | 0.31 |
| | glutamylglycine | | glutamyl | | | | | | | | |
| | gamma- | HMDB11171 | gamma- | 1.20 | 0.15 | 0.75 | 0.21 | 0.75 | 0.21 | 0.80 | 0.10 |
| | glutamyleucine | | glutamyl | | | | | | | | |
| | gamma- | | gamma- | 1.18 | 0.16 | 0.92 | 0.18 | 0.92 | 0.18 | 0.81 | 0.20 |
| | glutamylmethionine | | glutamyl | | | | | | | | |
| | gamma- | HMDB00594 | gamma- | 1.11 | 0.23 | 0.89 | 0.16 | 0.89 | 0.16 | 0.89 | 0.16 |
| | glutamylphenylalanine | | glutamyl | | | | | | | | |
| | gamma- | | gamma- | 1.09 | 0.17 | 0.97 | 0.11 | 0.97 | 0.11 | 0.89 | 0.20 |
| | glutamylthreonine* | | glutamyl | | | | | | | | |
| | gamma- | | gamma- | 1.08 | 0.11 | 0.82 | 0.33 | 0.82 | 0.33 | 0.91 | 0.29 |
| | glutamyltyrosine | | glutamyl | | | | | | | | |
| | glycylglycine | HMDB11733 | Dipeptide | 1.26 | 0.34 | 0.93 | 0.18 | 0.93 | 0.18 | 0.90 | 0.07 |
| | glycylleucine | HMDB00759 | Dipeptide | 1.17 | 0.21 | 0.90 | 0.09 | 0.90 | 0.09 | 0.97 | 0.15 |
| | glycylphenylalanine | | Dipeptide | 1.08 | 0.11 | 0.79 | 0.22 | 0.79 | 0.22 | 0.77 | 0.23 |
| | hemocamosine | HMDB00745 | Dipeptide | 0.82 | 0.17 | 1.07 | 0.13 | 1.07 | 0.13 | 1.36 | 0.17 |
| | | | derivative | | | | | | | | |
| | isoleucylglycine | | Dipeptide | 1.15 | 0.27 | 0.95 | 0.12 | 0.95 | 0.12 | 0.93 | 0.13 |
| | leucylglycine | | Dipeptide | 1.00 | 0.29 | 1.08 | 0.28 | 1.08 | 0.28 | 1.25 | 0.37 |
| | leucylserine | | Dipeptide | 1.56 | 0.24 | 0.76 | 0.19 | 0.76 | 0.19 | 0.62 | 0.22 |
| | phenylalanylglycine | | Dipeptide | 1.23 | 0.42 | 0.28 | 0.06 | 0.28 | 0.06 | 0.28 | 0.05 |
| | phenylalanylphenylalanine | | Dipeptide | 1.30 | 0.31 | 0.31 | 0.08 | 0.31 | 0.08 | 0.46 | 0.23 |
| | phenylalanylserine | | Dipeptide | 2.04 | 0.57 | 0.82 | 0.16 | 0.82 | 0.16 | 0.90 | 0.08 |
| | prolylmethionine | | Dipeptide | 1.35 | 0.34 | 0.85 | 0.14 | 0.85 | 0.14 | 0.86 | 0.19 |
| | theorylalanine | | Dipeptide | 0.98 | 0.27 | 0.98 | 0.30 | 0.98 | 0.30 | 1.11 | 0.37 |
| | tryptophylglycine | | Dipeptide | 1.37 | 0.33 | 0.91 | 0.13 | 0.91 | 0.13 | 0.81 | 0.21 |
| | tyrosylglycine | | Dipeptide | 1.77 | 0.29 | 0.62 | 0.12 | 0.62 | 0.12 | 0.64 | 0.14 |
| | tyrosyleucine | | Dipeptide | 1.55 | 0.29 | 0.63 | 0.09 | 0.63 | 0.09 | 0.68 | 0.17 |
| Xenobiotics | 2- | HMDB02039 | Chemical | 1.10 | 0.50 | 1.46 | 0.68 | 1.46 | 0.68 | 1.45 | 1.17 |
| | pyrrolidinone | | | | | | | | | | |
| | dihydrokaempferol | | Food | 1.00 | 0.00 | 1.00 | 0.00 | 1.00 | 0.00 | 1.00 | 0.00 |
| | | | component/Plant | | | | | | | | |
| | ergothioneine | HMDB03045 | Food | 1.30 | 0.23 | 0.89 | 0.25 | 0.89 | 0.25 | 0.91 | 0.30 |
| | | | component/Plant | | | | | | | | |
| | erythritol | HMDB02994 | Sugar, sugar | 0.95 | 0.23 | 1.13 | 0.19 | 1.13 | 0.19 | 1.06 | 0.21 |
| | | | substitute, starch | | | | | | | | |
| | glycerol 2-phosphate | HMDB02520 | Chemical | 1.12 | 0.35 | 0.90 | 0.24 | 0.90 | 0.24 | 0.97 | 0.32 |
| | hippurate | HMDB00714 | Benzoate | 0.74 | 0.44 | 0.90 | 0.32 | 0.90 | 0.32 | 1.08 | 0.69 |
| | | | metabolism | | | | | | | | |
| | ketamine | | Drug | 1.05 | 1.14 | 0.72 | 0.69 | 0.72 | 0.69 | 0.80 | 92 |
| | pentobarbital | | Drug | 0.71 | 0.61 | 1.02 | 0.62 | 1.02 | 0.62 | 1.33 | 0.92 |

Table 7 includes a complete non-lipid metabolomic profile at 8 weeks post-operation. Data represents averages of scaled metabolite amount±standard deviation. For each small molecule, the raw area counts were resealed to set the median metabolite amount equal to 1. The Human Metabolome Database (HMDB) identifier has been provided. The bold indicates averaged median values equal or lower than 1.5-fold from the median metabolite amount of 1, whereas italics indicates averaged median values equal or higher than 1.5-fold from the median metabolite amount.

TABLE 7

| | | | | Control: SCI | | Control: Sham | | O3PUFA: SCI | | O3PUFA: Sham | |
|---|---|---|---|---|---|---|---|---|---|---|---|
| | | | | Average | | Average | | Average | | Average | |
| | | | | Median | | Median | | Median | | Median | |
| | Metabolite | | | Metabolite | | Metabolite | | Metabolite | Standard | Metabolite | Standard |
| | 8 weeks | HMDB | Subpathway | Levels | Standard Deviation | Levels | Standard Deviation | Levels | Deviation | Levels | Deviation |
| Amino Acids | 2- | HMDB00510 | Lysine | 1.06 | 0.44 | 1.20 | 0.46 | 1.20 | 0.40 | 1.08 | 0.50 |
| | aminoadipate | | metabolism | | | | | | | | |
| | 2- | HMDB00650 | Butanoate | 1.64 | 0.80 | 0.89 | 0.68 | 0.89 | 0.68 | 0.79 | 0.48 |
| | aminobutyrate | | metabolism | | | | | | | | |
| | 2- | HMDB00008 | Cysteine, | 1.14 | 0.32 | 0.96 | 0.31 | 0.96 | 0.31 | 0.68 | 0.30 |
| | hydroxybutyrate | | methionine, | | | | | | | | |
| | (AHB) | | SAM, taurine | | | | | | | | |
| | | | metabolism | | | | | | | | |
| | 2- | HMDB00378 | Valine | 0.92 | 0.41 | 1.04 | 0.30 | 1.04 | 0.30 | 0.60 | 0.22 |
| | methylbutyroyl | | leucine and | | | | | | | | |
| | carnitine | | isoleucine | | | | | | | | |
| | | | metabolism | | | | | | | | |
| | 3-(4- | HMDB00755 | Phenylalanine | 0.91 | 0.22 | 0.92 | 0.23 | 0.92 | 0.23 | 0.90 | 0.26 |
| | hydroxyphenyl)lactate | | & tyrosine | | | | | | | | |
| | | | metabolism | | | | | | | | |
| | 3- | HMDB00336 | Valine, | 0.87 | 0.27 | 0.93 | 0.29 | 0.93 | 0.29 | 0.76 | 0.21 |
| | hydroxyisobutyrate | | leucine and | | | | | | | | |
| | | | isoleucine | | | | | | | | |
| | | | metabolism | | | | | | | | |
| | 3- | HMDB00272 | Glycine, | 0.95 | 0.25 | 1.17 | 0.42 | 1.17 | 0.42 | 0.61 | 0.29 |
| | phosphoserine | | serine and | | | | | | | | |
| | | | threonine | | | | | | | | |
| | | | metabolism | | | | | | | | |
| | 4- | HMDB03464 | Guanidino | 0.84 | 0.23 | 1.06 | 0.19 | 1.06 | 0.19 | 1.29 | 0.39 |
| | guanidinobutanoate | | and | | | | | | | | |
| | | | acetamido | | | | | | | | |
| | | | metabolism | | | | | | | | |
| | 5- | HMDB01173 | Polyamine | 0.88 | 0.13 | 1.05 | 0.19 | 1.05 | 0.19 | 1.04 | 0.14 |
| | methylthioadenosine (MTA) | | metabolism | | | | | | | | |
| | 5-oxoproline | HMDB00267 | Glutathione | 1.10 | 0.32 | 1.18 | 0.12 | 1.18 | 0.12 | 0.90 | 0.11 |
| | | | metabolism | | | | | | | | |
| | agmatine | HMDB01432 | Polyamine | 1.16 | 0.40 | 1.47 | 0.58 | 1.47 | 0.58 | 0.87 | 0.27 |
| | | | metabolism | | | | | | | | |
| | alanine | HMDB00161 | Alanine and | 1.21 | 0.22 | 1.29 | 0.21 | 1.29 | 0.21 | 0.85 | 0.08 |
| | | | aspartate | | | | | | | | |
| | | | metabolism | | | | | | | | |
| | arginine | HMDB00517 | Urea cycle; | 1.01 | 0.16 | 0.95 | 0.18 | 0.95 | 0.18 | 1.00 | 0.07 |
| | | | arginine-, | | | | | | | | |
| | | | proline-, | | | | | | | | |
| | | | metabolism | | | | | | | | |
| | asparagine | HMDB00168 | Alanine and | 1.00 | 0.22 | 0.75 | 0.26 | 0.75 | 0.29 | 1.10 | 0.15 |
| | | | aspartate | | | | | | | | |
| | | | metabolism | | | | | | | | |
| | aspartate | HMDB00191 | Alanine and | 0.82 | 0.23 | 0.79 | 0.12 | 0.79 | 0.12 | 1.27 | 0.15 |
| | | | aspartate | | | | | | | | |
| | | | metabolism | | | | | | | | |
| | beta-alanine | HMDB00056 | Alanine and | 1.08 | 0.42 | 0.92 | 0.37 | 0.92 | 0.37 | 1.02 | 0.52 |
| | | | aspartate | | | | | | | | |
| | | | metabolism | | | | | | | | |
| | betaine | HMDB00043 | Glycine, | 1.20 | 0.33 | 1.37 | 0.28 | 1.37 | 0.28 | 0.58 | 0.14 |
| | | | serine and | | | | | | | | |
| | | | threonine | | | | | | | | |
| | | | metabolism | | | | | | | | |
| | C- | | Tryptophan | 1.09 | 0.32 | 1.19 | 0.20 | 1.19 | 0.20 | 0.96 | 0.12 |
| | glycosyltryptophan* | | metabolism | | | | | | | | |
| | citrulline | HMDB00904 | Urea cycle; | 1.19 | 0.25 | 1.33 | 0.25 | 1.33 | 0.25 | 0.30 | 0.03 |
| | | | arginine-, | | | | | | | | |
| | | | proline-, | | | | | | | | |
| | | | metabolism | | | | | | | | |
| | creatine | HMDB00064 | Creatine | 0.90 | 0.16 | 0.95 | 0.07 | 0.95 | 0.07 | 1.07 | 0.07 |
| | | | metabolism | | | | | | | | |
| | creatinine | HMDB00562 | Creatine | 0.95 | 0.21 | 1.03 | 0.14 | 1.03 | 0.14 | 1.01 | 0.22 |
| | | | metabolism | | | | | | | | |
| | cystathionine | HMDB00099 | Cysteine, | 0.91 | 0.16 | 1.01 | 0.25 | 1.01 | 0.25 | 1.10 | 0.21 |
| | | | methionine, | | | | | | | | |
| | | | SAM, taurine | | | | | | | | |
| | | | metabolism | | | | | | | | |
| | cysteine | HMDB00574 | Cysteine, | 1.11 | 0.40 | 1.38 | 0.39 | 1.38 | 0.39 | 0.83 | 0.15 |
| | | | methionine, | | | | | | | | |
| | | | SAM, taurine | | | | | | | | |
| | | | metabolism | | | | | | | | |
| | cysteine- | HMDB00658 | Glutathione | 1.33 | 0.39 | 1.40 | 0.16 | 1.40 | 0.16 | 0.83 | 0.08 |
| | glutathione | | metabolism | | | | | | | | |
| | disulfide | | | | | | | | | | |
| | cystine | HMDB00192 | Cysteine, | 1.34 | 0.63 | 1.33 | 0.35 | 1.33 | 0.35 | 0.75 | 0.12 |
| | | | methionine, | | | | | | | | |
| | | | SAM, taurine | | | | | | | | |
| | | | metabolism | | | | | | | | |
| | gamma- | HMDB00112 | Glutamate | 0.87 | 0.32 | 0.73 | 0.22 | 0.73 | 0.22 | 1.44 | 0.46 |
| | aminobutyrate | | metabolism | | | | | | | | |
| | (GABA) | | | | | | | | | | |
| | glutamate | HMDB03339 | Glutamate | 0.80 | 0.14 | 0.86 | 0.09 | 0.86 | 0.09 | 1.19 | 0.10 |
| | | | metabolism | | | | | | | | |
| | glutamine | HMDB00641 | Glutamate | 0.96 | 0.21 | 1.08 | 0.16 | 1.08 | 0.16 | 1.00 | 0.12 |
| | | | metabolism | | | | | | | | |
| | glutathione, | HMDB03337 | Glutathione | 1.14 | 0.39 | 1.54 | 0.21 | 1.54 | 0.21 | 0.70 | 0.15 |
| | oxidized | | metabolism | | | | | | | | |
| | (GSSG) | | | | | | | | | | |
| | glutathione, | HMDB00125 | Glutathione | 0.89 | 0.14 | 1.25 | 0.30 | 1.25 | 0.30 | 0.99 | 0.17 |
| | reduced | | metabolism | | | | | | | | |
| | (GSH) | | | | | | | | | | |
| | glycine | HMDB00123 | Glycine, | 0.77 | 0.22 | 0.81 | 0.14 | 0.81 | 0.14 | 1.23 | 0.14 |
| | | | serine and | | | | | | | | |
| | | | threonine | | | | | | | | |
| | | | metabolism | | | | | | | | |
| | histidine | HMDB00177 | Histidine | 1.14 | 0.16 | 1.13 | 0.13 | 1.13 | 0.13 | 0.73 | 0.04 |
| | | | metabolism | | | | | | | | |
| | hydroxyisovaleroyl | | Valine, | 1.12 | 0.41 | 1.37 | 0.24 | 1.37 | 0.24 | 0.85 | 0.22 |
| | carnitine | | leucine and | | | | | | | | |
| | | | isoleucine | | | | | | | | |
| | | | metabolism | | | | | | | | |
| | hypotaurine | HMDB00965 | Cysteine, | 1.50 | 0.67 | 1.58 | 0.61 | 1.58 | 0.61 | 0.84 | 0.35 |
| | | | methionine, | | | | | | | | |
| | | | SAM, taurine | | | | | | | | |
| | | | metabolism | | | | | | | | |
| | isoleucine | HMDB00172 | Valine, | 1.23 | 0.24 | 1.13 | 0.13 | 1.13 | 0.13 | 0.69 | 0.06 |
| | | | leucine and | | | | | | | | |
| | | | isoleucine | | | | | | | | |
| | | | metabolism | | | | | | | | |
| | isovalerylcamitine | HMDB00688 | Valine, | 1.04 | 0.42 | 1.20 | 0.39 | 1.20 | 0.39 | 0.89 | 0.28 |
| | | | leucine and | | | | | | | | |
| | | | isoleucine | | | | | | | | |
| | | | metabolism | | | | | | | | |
| | kynurenine | HMDB00684 | Tryptophan | 0.87 | 0.66 | 0.74 | 0.57 | 0.74 | 0.57 | 0.96 | 0.58 |
| | | | metabolism | | | | | | | | |
| | leucine | HMDB00687 | Valine, | 1.21 | 0.24 | 1.14 | 0.13 | 1.14 | 0.13 | 0.71 | 0.08 |
| | | | leucine and | | | | | | | | |
| | | | isoleucine | | | | | | | | |
| | | | metabolism | | | | | | | | |
| | lysine | HMDB00182 | Lysine | 1.22 | 0.30 | 1.34 | 0.15 | 1.34 | 0.15 | 0.91 | 0.09 |
| | | | metabolism | | | | | | | | |
| | methionine | HMDB00696 | Cysteine, | 1.14 | 0.18 | 1.11 | 0.12 | 1.11 | 0.12 | 0.76 | 0.08 |
| | | | methionine, | | | | | | | | |
| | | | SAM, taurine | | | | | | | | |
| | | | metabolism | | | | | | | | |
| | N-acetyl- | HMDB01067 | Glutamate | 0.79 | 0.19 | 0.81 | 0.10 | 0.81 | 0.10 | 1.77 | 0.17 |
| | aspartyl- | | metabolism | | | | | | | | |
| | glutamate | | | | | | | | | | |
| | (NAAG) | | | | | | | | | | |
| | N- | HMDB00766 | Alanine and | 1.05 | 0.32 | 0.92 | 0.14 | 0.92 | 0.14 | 1.10 | 0.16 |
| | acetylalanine | | aspartate | | | | | | | | |
| | | | metabolism | | | | | | | | |
| | N- | HMDB00812 | Alanine and | 0.77 | 0.22 | 0.82 | 0.09 | 0.82 | 0.09 | 1.55 | 0.07 |
| | acetylaspartate | | aspartate | | | | | | | | |
| | (NAA) | | metabolism | | | | | | | | |
| | N- | HMDB01138 | Glutamate | 0.77 | 0.21 | 0.73 | 0.15 | 0.73 | 0.15 | 1.47 | 0.19 |
| | acetylglutamate | | metabolism | | | | | | | | |
| | N- | HMDB11745 | Cysteine, | 1.40 | 0.31 | 1.22 | 0.17 | 1.22 | 0.17 | 0.49 | 0.08 |
| | acetylmethionine | | methionine, | | | | | | | | |
| | | | SAM, taurine | | | | | | | | |
| | | | metabolism | | | | | | | | |
| | N- | | Trytophan | 1.00 | 0.00 | 1.00 | 0.00 | 1.00 | 0.00 | 1.00 | 0.00 |
| | acetyltryptophan | | metabolism | | | | | | | | |
| | N2- | HMDB00446 | Lysine | 0.93 | 0.25 | 1.38 | 0.38 | 1.38 | 0.38 | 1.03 | 0.46 |
| | acetyllysine | | metabolism | | | | | | | | |
| | ophthalmate | HMDB05765 | Glutathione | 1.21 | 0.61 | 0.95 | 0.58 | 0.95 | 0.58 | 0.93 | 0.41 |
| | | | metabolism | | | | | | | | |
| | ornithine | HMDB03374 | Urea cycle; | 1.07 | 0.31 | 1.19 | 0.20 | 1.19 | 0.20 | 0.87 | 0.22 |
| | | | arginine-, | | | | | | | | |
| | | | proline-, | | | | | | | | |
| | | | metabolism | | | | | | | | |
| | phenylalanine | HMDB00159 | Phenylalanine | 1.21 | 0.22 | 1.13 | 0.14 | 1.13 | 0.14 | 0.86 | 0.09 |
| | | | & tyrosine | | | | | | | | |
| | | | metabolism | | | | | | | | |
| | pipecolate | HMDB00070 | Lysine | 1.25 | 0.38 | 1.28 | 0.25 | 1.28 | 0.25 | 0.93 | 0.24 |
| | | | metabolism | | | | | | | | |
| | proline | HMDB00162 | Urea cycle; | 1.13 | 0.11 | 1.15 | 0.15 | 1.15 | 0.15 | 0.79 | 0.05 |
| | | | arginine-, | | | | | | | | |
| | | | proline-, | | | | | | | | |
| | | | metabolism | | | | | | | | |
| | putrescine | HMDB01414 | Polyamine | 1.90 | 0.71 | 1.99 | 0.73 | 1.99 | 0.73 | 0.62 | 0.19 |
| | | | metabolism | | | | | | | | |
| | S- | HMDB00939 | Cysteine, | 0.90 | 0.23 | 0.88 | 0.15 | 0.88 | 0.15 | 1.10 | 0.15 |
| | adenosylhomocysteine | | methionine, | | | | | | | | |
| | (SAH) | | SAM, taurine | | | | | | | | |
| | | | metabolism | | | | | | | | |
| | S- | HMDB02108 | Cysteine, | 0.86 | 0.42 | 0.98 | 0.33 | 0.95 | 0.33 | 0.69 | 0.16 |
| | methylcysteine | | methionine, | | | | | | | | |
| | | | SAM, taurine | | | | | | | | |
| | | | metabolism | | | | | | | | |
| | saccharopine | HMDB00279 | Lysine | 0.65 | 0.01 | 0.75 | 0.21 | 0.75 | 0.21 | 0.91 | 0.40 |
| | | | metabolism | | | | | | | | |
| | sarcosine (N- | HMDB00271 | Glycine, | 1.00 | 0.37 | 1.05 | 0.32 | 1.05 | 0.32 | 1.07 | 0.13 |
| | Methylglycine) | | serine and | | | | | | | | |
| | | | threonine | | | | | | | | |
| | | | metabolism | | | | | | | | |
| | serine | HMDB03406 | Glycine, | 1.16 | 0.23 | 1.22 | 0.19 | 1.22 | 0.19 | 0.87 | 0.10 |
| | | | serine and | | | | | | | | |
| | | | threonine | | | | | | | | |
| | | | metabolism | | | | | | | | |
| | spermidine | HMDB01257 | Polyamine | 0.88 | 0.25 | 0.97 | 0.19 | 0.97 | 0.19 | 1.00 | 0.12 |
| | | | metabolism | | | | | | | | |
| | taurine | HMDB00251 | Cysteine, | 1.24 | 0.30 | 1.47 | 0.28 | 1.47 | 0.28 | 0.76 | 0.16 |
| | | | methionine, | | | | | | | | |
| | | | SAM, taurine | | | | | | | | |
| | | | metabolism | | | | | | | | |
| | threonine | HMDB00167 | Glycine, | 1.04 | 0.19 | 1.04 | 0.15 | 1.04 | 0.15 | 0.86 | 0.14 |
| | | | serine and | | | | | | | | |
| | | | threonine | | | | | | | | |
| | | | metabolism | | | | | | | | |
| | trans-4- | HMDB00725 | Urea cycle; | 0.87 | 0.61 | 0.75 | 0.59 | 0.75 | 0.59 | 0.29 | 0.11 |
| | hydroxyproline | | arginine-, | | | | | | | | |
| | | | proline-, | | | | | | | | |
| | | | metabolism | | | | | | | | |
| | tryptophan | HMDB00929 | Tryptophan | 1.10 | 0.19 | 1.10 | 0.13 | 1.10 | 0.13 | 0.97 | 0.07 |
| | | | metabolism | | | | | | | | |
| | tyrosine | HMDB00158 | Phenylalanine | 1.17 | 0.21 | 1.18 | 0.14 | 1.18 | 0.14 | 0.64 | 0.06 |
| | | | & tyrosine | | | | | | | | |
| | | | metabolism | | | | | | | | |
| | urea | HMDB00294 | Urea cycle; | 0.76 | 0.49 | 0.98 | 0.54 | 0.98 | 0.54 | 1.24 | 0.47 |
| | | | arginine-, | | | | | | | | |
| | | | proline-, | | | | | | | | |
| | | | metabolism | | | | | | | | |
| | valine | HMDB00883 | Valine, | 1.21 | 0.27 | 1.16 | 0.15 | 1.16 | 0.15 | 0.69 | 0.07 |
| | | | leucine and | | | | | | | | |
| | | | isoleucine | | | | | | | | |
| | | | metabolism | | | | | | | | |
| Carbohydrates | 1,5- | HMDB02712 | Glycolysis, | 1.14 | 0.52 | 1.04 | 0.28 | 1.04 | 0.28 | 0.83 | 0.21 |
| | anhydroglucitol | | gluconeogenesis, | | | | | | | | |
| | (1,5-AG) | | pyruvate | | | | | | | | |
| | | | metabolism | | | | | | | | |
| | 1,6- | HMDB00640 | Glycolysis, | 0.57 | 0.27 | 0.90 | 0.55 | 0.90 | 0.55 | 0.64 | 0.34 |
| | anhydroglucose | | gluconeogenesis, | | | | | | | | |
| | | | pyruvate | | | | | | | | |
| | | | metabolism | | | | | | | | |
| | arabinose | HMDB00646 | Nucleotide | 0.52 | 0.29 | 0.69 | 0.53 | 0.69 | 0.53 | 0.66 | 0.46 |
| | | | sugars, | | | | | | | | |
| | | | pentose | | | | | | | | |
| | | | metabolism | | | | | | | | |
| | arabitol | HMDB01861 | Nucleotide | 0.91 | 0.26 | 1.02 | 0.36 | 1.02 | 0.36 | [symbol] | 0.31 |
| | | | sugars, | | | | | | | | |
| | | | pentose | | | | | | | | |
| | | | metabolism | | | | | | | | |
| | dihydroxyacetone | HMDB01473 | Glycolysis, | 1.08 | 0.29 | 1.07 | 0.28 | 1.07 | 0.28 | 0.88 | 0.17 |
| | phosphate | | gluconeogenesis, | | | | | | | | |
| | (DHAP) | | pyruvate | | | | | | | | |
| | | | metabolism | | | | | | | | |
| | erythronate* | HMDB00613 | Aminosugars | 1.12 | 0.36 | 1.15 | 0.23 | 1.15 | 0.23 | 0.92 | 0.17 |
| | | | metabolism | | | | | | | | |
| | fructose | HMDB00660 | Fructose, | 1.28 | 0.47 | 1.38 | 0.31 | 1.38 | 0.31 | 0.69 | 0.15 |
| | | | mannose, | | | | | | | | |
| | | | galactose, | | | | | | | | |
| | | | starch, and | | | | | | | | |
| | | | sucrose | | | | | | | | |
| | | | metabolism | | | | | | | | |
| | fructose 1- | HMDB01076 | Glycolysis, | 1.24 | 0.63 | 1.45 | 0.61 | 1.45 | 0.61 | 0.67 | 0.22 |
| | phosphate | | gluconeogenesis, | | | | | | | | |
| | | | pyruvate | | | | | | | | |
| | | | metabolism | | | | | | | | |
| | fructose-6- | HMDB00124 | Glycolysis, | 1.46 | 0.49 | 1.72 | 0.43 | 1.72 | 0.43 | 0.25 | 0.08 |
| | phosphate | | gluconeogenesis, | | | | | | | | |
| | | | pyruvate | | | | | | | | |
| | | | metabolism | | | | | | | | |
| | galactose | HMDB00143 | Fructose, | 1.31 | 0.37 | 1.39 | 0.27 | 1.39 | 0.27 | 0.29 | 0.09 |
| | | | mannose, | | | | | | | | |
| | | | galactose, | | | | | | | | |
| | | | starch, and | | | | | | | | |
| | | | sucrose | | | | | | | | |
| | | | metabolism | | | | | | | | |
| | glucose | HMDB00122 | Glycolysis, | 1.61 | 0.72 | 2.07 | 0.70 | 2.07 | 0.70 | 0.13 | 0.04 |
| | | | gluconeogenesis, | | | | | | | | |
| | | | pyruvate | | | | | | | | |
| | | | metabolism | | | | | | | | |
| | glucose-6- | HMDB01401 | Glycolysis, | 1.44 | 0.47 | 1.69 | 0.41 | 1.69 | 0.41 | 0.23 | 0.07 |
| | phosphate | | gluconeogenesis, | | | | | | | | |
| | (G6P) | | pyruvate | | | | | | | | |
| | | | metabolism | | | | | | | | |
| | glycerate | HMDB00139 | Glycolysis, | 0.86 | 0.23 | 1.13 | 0.48 | 1.13 | 0.48 | 0.67 | 0.34 |
| | | | gluconeogenesis, | | | | | | | | |
| | | | pyruvate | | | | | | | | |
| | | | metabolism | | | | | | | | |
| | Isobar: | | Glycolysis, | 0.97 | 0.22 | 0.91 | 0.13 | 0.91 | 0.13 | 0.98 | 0.16 |
| | fructose 1,6- | | gluconeogenesis, | | | | | | | | |
| | diphosphate, | | pyruvate | | | | | | | | |
| | glucose 1,6- | | metabolism | | | | | | | | |
| | diphosphate, | | | | | | | | | | |
| | myo-inositol | | | | | | | | | | |
| | 1,4 or 1 | | | | | | | | | | |
| | Isobar: | | Nucleotide | 1.40 | 0.57 | 1.57 | 0.41 | 1.57 | 0.41 | 0.55 | 0.11 |
| | ribulose 5- | | sugars, | | | | | | | | |
| | phosphate, | | pentose | | | | | | | | |
| | xylulose 5- | | metabolism | | | | | | | | |
| | phosphate | | | | | | | | | | |
| | lactate | HMDB00190 | Glycolysis, | 1.01 | 0.24 | 1.09 | 0.18 | 1.09 | 0.18 | [symbol] | 0.13 |
| | | | gluconeogenesis, | | | | | | | | |
| | | | pyruvate | | | | | | | | |
| | | | metabolism | | | | | | | | |
| | maltose | HMDB00163 | Fructose, | 1.30 | 0.41 | 2.21 | 1.04 | 2.21 | 1.04 | 0.39 | 0.07 |
| | | | mannose, | | | | | | | | |
| | | | galactose, | | | | | | | | |
| | | | starch, and | | | | | | | | |
| | | | sucrose | | | | | | | | |
| | | | metabolism | | | | | | | | |
| | maltotriose | HMDB01262 | Fructose, | 0.64 | 0.18 | 1.40 | 0.70 | 1.40 | 0.70 | 0.52 | 0.00 |
| | | | mannose, | | | | | | | | |
| | | | galactose, | | | | | | | | |
| | | | starch, and | | | | | | | | |
| | | | sucrose | | | | | | | | |
| | | | metabolism | | | | | | | | |
| | mannitol | HMDB00765 | Fructose, | 0.98 | 0.46 | 0.88 | 0.49 | 0.88 | 0.49 | 0.75 | 0.26 |
| | | | mannose, | | | | | | | | |
| | | | galactose, | | | | | | | | |
| | | | starch, and | | | | | | | | |
| | | | sucrose | | | | | | | | |
| | | | metabolism | | | | | | | | |
| | mannose | HMDB00169 | Fructose, | 1.39 | 0.37 | 1.52 | 0.27 | 1.52 | 0.27 | 0.29 | 0.03 |
| | | | mannose, | | | | | | | | |
| | | | galactose, | | | | | | | | |
| | | | starch, and | | | | | | | | |
| | | | sucrose | | | | | | | | |
| | | | metabolism | | | | | | | | |
| | mannose-6- | HMDB01078 | Fructose, | 1.43 | 0.45 | 1.76 | 0.43 | 1.76 | 0.43 | 0.25 | 0.07 |
| | phosphate | | mannose, | | | | | | | | |
| | | | galactose, | | | | | | | | |
| | | | starch, and | | | | | | | | |
| | | | sucrose | | | | | | | | |
| | | | metabolism | | | | | | | | |
| | N- | HMDB02817 | Aminosugars | 0.99 | 0.43 | 1.35 | 0.42 | 1.35 | 0.42 | 0.83 | 0.36 |
| | acetylglucosamine | | metabolism | | | | | | | | |
| | 6- | | | | | | | | | | |
| | phosphate | | | | | | | | | | |
| | N- | HMDB00230 | Aminosugars | 1.08 | 0.26 | 1.09 | 0.15 | 1.09 | 0.15 | 0.83 | 0.11 |
| | acetylneuraminate | | metabolism | | | | | | | | |
| | ribose | HMDB00283 | Nucleotide | 0.90 | 0.32 | 1.09 | 0.35 | 1.09 | 0.35 | 1.01 | 0.13 |
| | | | sugars, | | | | | | | | |
| | | | pentose | | | | | | | | |
| | | | metabolism | | | | | | | | |
| | ribulose | HMDB00621, | Nucleotide | 1.31 | 0.55 | 1.34 | 0.70 | 1.34 | 0.70 | 0.62 | 0.32 |
| | | HMDB03371 | sugars, | | | | | | | | |
| | | | pentose | | | | | | | | |
| | | | metabolism | | | | | | | | |
| | sedoheptulose- | HMDB01068 | Nucleotide | 1.44 | 0.64 | 1.52 | 0.53 | 1.52 | 0.53 | 0.24 | 0.11 |
| | 7- | | sugars, | | | | | | | | |
| | phosphate | | pentose | | | | | | | | |
| | | | metabolism | | | | | | | | |
| | sorbitol | HMDB00247 | Fructose, | 1.19 | 0.43 | 1.23 | 0.36 | 1.23 | 0.36 | 0.78 | 0.23 |
| | | | mannose, | | | | | | | | |
| | | | galactose, | | | | | | | | |
| | | | starch, and | | | | | | | | |
| | | | sucrose | | | | | | | | |
| | | | metabolism | | | | | | | | |
| Cofactors and vitamins | 3′- | HMDB01373 | Pantothenate | 0.73 | 0.26 | 0.79 | 0.27 | 0.79 | 0.27 | 1.30 | 0.35 |
| | dephosphocoenzyme A | | and CoA | | | | | | | | |
| | | | metabolism | | | | | | | | |
| | acetyl CoA | HMDB01206 | Pantothenate | 0.82 | 0.19 | 0.89 | 0.16 | 0.89 | 0.16 | 1.30 | 0.28 |
| | | | and CoA | | | | | | | | |
| | | | metabolism | | | | | | | | |
| | alpha- | HMDB01893 | Tocopherol | 1.19 | 0.14 | 1.29 | 0.26 | 1.29 | 0.26 | 0.77 | 0.15 |
| | tocopherol | | metabolism | | | | | | | | |
| | ascorbate | HMDB00044 | Ascorbate | 1.19 | 0.24 | 1.32 | 0.28 | 1.32 | 0.28 | 0.85 | 0.11 |
| | (Vitamin C) | | and aldarate | | | | | | | | |
| | | | metabolism | | | | | | | | |
| | coenzyme A | HMDB01423 | Pantothenate | 0.73 | 0.20 | 0.83 | 0.16 | 0.83 | 0.16 | 1.48 | 0.27 |
| | | | and CoA | | | | | | | | |
| | | | metabolism | | | | | | | | |
| | dihydrobiopterin | HMDB00038 | Folate | 1.18 | 0.37 | 1.40 | 0.47 | 1.40 | 0.47 | 0.53 | 0.14 |
| | | | metabolism | | | | | | | | |
| | flavin adenine | HMDB01248 | Riboflavin | 1.09 | 0.20 | 1.13 | 0.16 | 1.13 | 0.16 | 0.83 | 0.11 |
| | dinucleotide | | metabolism | | | | | | | | |
| | (FAD) | | | | | | | | | | |
| | heme | | Hemoglobin | 1.01 | 0.37 | 1.07 | 0.29 | 1.07 | 0.29 | 0.96 | 0.42 |
| | | | and porphyrin | | | | | | | | |
| | | | metabolism | | | | | | | | |
| | nicotinamide | HMDB01406 | Nicotinate and | 0.97 | 0.22 | 0.99 | 0.19 | 0.99 | 0.19 | 1.00 | 0.20 |
| | | | nicotinamide | | | | | | | | |
| | | | metabolism | | | | | | | | |
| | pantothenate | HMDB00210 | Pantothenate | 1.24 | 0.24 | 1.36 | 0.28 | 1.36 | 0.28 | 0.82 | 0.17 |
| | | | and CoA | | | | | | | | |
| | | | metabolism | | | | | | | | |
| | phosphopantetheine | HMDB01416 | Pantothenate | 1.00 | 0.19 | 1.20 | 0.33 | 1.20 | 0.33 | 0.94 | 0.16 |
| | | | and CoA | | | | | | | | |
| | | | metabolism | | | | | | | | |
| | riboflavin | HMDB00244 | Riboflavin | 1.07 | 0.05 | 1.08 | 0.23 | 1.08 | 0.23 | 0.80 | 0.15 |
| | (Vitamin B2) | | metabolism | | | | | | | | |
| Energy | acetylphosphate | HMDB01494 | Oxidative | 0.98 | 0.20 | 1.11 | 0.18 | 1.11 | 0.18 | 0.97 | 0.04 |
| | | | phosphorylation | | | | | | | | |
| | citrate | HMDB00094 | Krebs cycle | 1.15 | 0.32 | 1.49 | 0.43 | 1.49 | 0.43 | 0.79 | 0.18 |
| | lumarate | HMDB00134 | Krebs cycle | 0.95 | 0.33 | 1.14 | 0.35 | 1.14 | 0.35 | 1.08 | 0.31 |
| | malate | HMDB00156 | Krebs cycle | 0.94 | 0.24 | 1.05 | 0.20 | 1.05 | 0.20 | 0.94 | 0.07 |
| | phosphate | HMDB01429 | Oxidative | 0.97 | 0.07 | 0.98 | 0.05 | 0.98 | 0.05 | 1.03 | 0.04 |
| | | | phosphorylation | | | | | | | | |
| | pyrophosphate | HMDB00250 | Oxidative | 1.04 | 0.30 | 1.15 | 0.27 | 1.15 | 0.27 | 0.89 | 0.20 |
| | (PPi) | | phosphorylation | | | | | | | | |
| Nucleotides | adenine | HMDB00034 | Purine | 0.81 | 0.18 | 0.86 | 0.13 | 0.86 | 0.13 | 1.35 | 0.28 |
| | | | metabolism, | | | | | | | | |
| | | | adenine | | | | | | | | |
| | | | containing | | | | | | | | |
| | adenosine | HMDB00050 | Purine | 0.73 | 0.21 | 0.67 | 0.16 | 0.67 | 0.18 | 2.87 | 0.63 |
| | | | metabolism, | | | | | | | | |
| | | | adenine | | | | | | | | |
| | | | containing | | | | | | | | |
| | adenosine 2′- | HMDB11617 | Purine | 1.42 | 0.48 | 1.35 | 0.22 | 1.35 | 0.22 | 0.58 | 0.10 |
| | monophosphate | | metabolism, | | | | | | | | |
| | (2′-AMP) | | adenine | | | | | | | | |
| | | | containing | | | | | | | | |
| | adenosine 3′- | HMDB03540 | Purine | 1.03 | 0.20 | 1.09 | 0.18 | 1.09 | 0.18 | 0.87 | 0.19 |
| | monophosphate | | metabolism, | | | | | | | | |
| | (3′-AMP) | | adenine | | | | | | | | |
| | | | containing | | | | | | | | |
| | adenosine 5′- | HMDB00045 | Purine | 1.04 | 0.32 | 1.05 | 0.23 | 1.05 | 0.23 | 0.93 | 0.36 |
| | monophosphate | | metabolism, | | | | | | | | |
| | (AMP) | | adenine | | | | | | | | |
| | | | containing | | | | | | | | |
| | alfantoin | HMDB00462 | Purine | 0.70 | 0.39 | 0.92 | 0.51 | 0.92 | 0.51 | 0.99 | 0.95 |
| | | | metabolism, | | | | | | | | |
| | | | urate | | | | | | | | |
| | | | metabolism | | | | | | | | |
| | arabinosylhypoxanthine | | Purine | 0.76 | 0.38 | 0.71 | 0.17 | 0.71 | 0.17 | 0.63 | 0.00 |
| | | | metabolism, | | | | | | | | |
| | | | (hypo)xanthine/ | | | | | | | | |
| | | | inosine | | | | | | | | |
| | | | containing | | | | | | | | |
| | cytidine | HMDB00089 | Pyrimidine | 1.12 | 0.16 | 1.35 | 0.16 | 1.35 | 0.16 | 0.86 | 0.06 |
| | | | metabolism, | | | | | | | | |
| | | | cytidine | | | | | | | | |
| | | | containing | | | | | | | | |
| | cytidine 3′- | | Pyrimidine | 1.12 | 0.15 | 1.02 | 0.18 | 1.02 | 0.18 | 0.97 | 0.15 |
| | monophosphate | | metabolism, | | | | | | | | |
| | (3′-CMP) | | cytidine | | | | | | | | |
| | | | containing | | | | | | | | |
| | guanosine | HMDB00133 | Purine | 0.65 | 0.28 | 0.70 | 0.18 | 0.70 | 0.18 | 1.76 | 0.30 |
| | | | metabolism, | | | | | | | | |
| | | | guanine | | | | | | | | |
| | | | containing | | | | | | | | |
| | hypoxanthine | HMDB00157 | Purine | 1.13 | 0.32 | 1.14 | 0.19 | 1.14 | 0.19 | 0.94 | 0.10 |
| | | | metabolism, | | | | | | | | |
| | | | (hypo)xanthine/ | | | | | | | | |
| | | | inosine | | | | | | | | |
| | | | containing | | | | | | | | |
| | inosine | | Purine | 0.88 | 0.12 | 0.95 | 0.08 | 0.95 | 0.08 | 1.05 | 0.08 |
| | | | metabolism, | | | | | | | | |
| | | | (hypo)xanthine/ | | | | | | | | |
| | | | inosine | | | | | | | | |
| | | | containing | | | | | | | | |
| | methylphosphate | | Purine and | 1.00 | 0.33 | 1.26 | 0.20 | 1.26 | 0.20 | 0.95 | 0.09 |
| | | | pyrimidine | | | | | | | | |
| | | | metabolism | | | | | | | | |
| | N1- | HMDB03331 | Purine | 0.94 | 0.26 | 1.02 | 0.18 | 1.02 | 0.18 | 0.71 | 0.23 |
| | methyladenosine | | metabolism, | | | | | | | | |
| | | | adenine | | | | | | | | |
| | | | containing | | | | | | | | |
| | pseudouridine | HMDB00767 | Pyrimidine | 1.06 | 0.17 | 1.02 | 0.15 | 1.02 | 0.15 | 0.99 | 0.14 |
| | | | metabolism, | | | | | | | | |
| | | | uracil | | | | | | | | |
| | | | containing | | | | | | | | |
| | uracil | HMDB00300 | Pyrimidine | 1.35 | 0.38 | 1.26 | 0.24 | 1.26 | 0.24 | 0.85 | 0.08 |
| | | | metabolism, | | | | | | | | |
| | | | uracil | | | | | | | | |
| | | | containing | | | | | | | | |
| | urate | HMDB00289 | Purine | 1.11 | 0.30 | 1.53 | 0.48 | 1.53 | 0.48 | 0.77 | 0.31 |
| | | | metabolism, | | | | | | | | |
| | | | urate | | | | | | | | |
| | | | metabolism | | | | | | | | |
| | uridine | HMDB00298 | Pyrimidine | 1.01 | 0.17 | 1.16 | 0.14 | 1.16 | 0.14 | 0.94 | 0.11 |
| | | | metabolism, | | | | | | | | |
| | | | uracil | | | | | | | | |
| | | | containing | | | | | | | | |
| | xanthine | HMDB00292 | Purine | 0.91 | 0.18 | 1.02 | 0.18 | 1.02 | 0.18 | 0.96 | 0.08 |
| | | | metabolism, | | | | | | | | |
| | | | (hypo)xanthine/ | | | | | | | | |
| | | | inosine | | | | | | | | |
| | | | containing | | | | | | | | |
| | xanthosine | HMDB00299 | Purine | 1.25 | 0.42 | 1.54 | 0.36 | 1.54 | 0.36 | 0.81 | 0.14 |
| | | | metabolism, | | | | | | | | |
| | | | (hypo)xanthine/ | | | | | | | | |
| | | | inosine | | | | | | | | |
| | | | containing | | | | | | | | |
| Peptides | anserine | HMDB00194 | Dipeptide | 1.56 | 1.31 | 0.78 | 0.46 | 0.78 | 0.46 | 0.95 | 0.40 |
| | | | derivative | | | | | | | | |
| | gamma- | | gamma- | 0.98 | 0.33 | 1.01 | 0.29 | 1.01 | 0.29 | 1.09 | 0.12 |
| | glutamylalanine | | glutamyl | | | | | | | | |
| | gamma- | | gamma- | 0.74 | 0.23 | 0.76 | 0.12 | 0.76 | 0.12 | 1.57 | 0.11 |
| | glutamylglutamate | | glutamyl | | | | | | | | |
| | gamma- | HMDB11738 | gamma- | 0.84 | 0.13 | 0.97 | 0.15 | 0.97 | 0.15 | 1.14 | 0.16 |
| | glutamylglutamine | | glutamyl | | | | | | | | |
| | gamma- | HMDB11667 | gamma- | 0.48 | 0.34 | 0.60 | 0.36 | 0.60 | 0.36 | 1.18 | 0.23 |
| | glutamylglycine | | glutamyl | | | | | | | | |
| | gamma- | HMDB11171 | gamma- | 1.24 | 0.33 | 1.11 | 0.32 | 1.11 | 0.32 | 0.99 | 0.12 |
| | glutamylleucine | | glutamyl | | | | | | | | |
| | gamma- | | gamma- | 1.07 | 0.28 | 0.96 | 0.25 | 0.96 | 0.25 | 0.92 | 0.14 |
| | glutamylmethionine | | glutamyl | | | | | | | | |
| | gamma- | HMDB00594 | gamma- | 1.22 | 0.55 | 0.94 | 0.19 | 0.94 | 0.19 | 0.97 | 0.20 |
| | glutamylphenylalanine | | glutamyl | | | | | | | | |
| | gamma- | | gamma- | 0.93 | 0.24 | 1.07 | 0.36 | 1.07 | 0.36 | 1.18 | 0.42 |
| | glutamylthreonine* | | glutamyl | | | | | | | | |
| | glycylglycine | HMDB11733 | Dipeptide | 0.95 | 0.38 | 1.08 | 0.30 | 1.08 | 0.30 | 0.86 | 0.50 |
| | glycylleucine | HMDB00759 | Dipeptide | 1.05 | 0.31 | 1.03 | 0.25 | 1.03 | 0.25 | 0.91 | 0.21 |
| | homocarnosine | HMDB00745 | Dipeptide | 0.87 | 0.18 | 1.01 | 0.19 | 1.01 | 0.19 | 1.22 | 0.28 |
| | | | derivative | | | | | | | | |
| | isoleucylglycine | | Dipeptide | 1.09 | 0.35 | 1.23 | 0.31 | 1.23 | 0.31 | 0.93 | 0.33 |
| | leucylglycine | | Dipeptide | 1.22 | 0.64 | 1.23 | 0.38 | 1.23 | 0.38 | 0.73 | 0.27 |
| | leucylserine | | Dipeptide | 1.32 | 0.65 | 1.50 | 0.35 | 1.50 | 0.35 | 0.85 | 0.20 |
| Xenobiotics | 2-ethylhexanoate | | Chemical | 0.97 | 0.23 | 0.93 | 0.13 | 0.93 | 0.13 | 1.04 | 0.16 |
| | (isobar with | | | | | | | | | | |
| | 2-propylperitanoate) | | | | | | | | | | |
| | 2- | HMDB02039 | Chemical | 0.85 | 0.42 | 1.07 | 0.34 | 1.07 | 0.34 | 1.28 | 0.47 |
| | pyrrolidinone | | | | | | | | | | |
| | ergothioneine | HMDB03045 | Food | 1.28 | 0.28 | 0.66 | 0.12 | 0.66 | 0.12 | 0.94 | 0.71 |
| | | | component/Plant | | | | | | | | |
| | erythritol | HMDB02994 | Sugar, sugar | 0.87 | 0.32 | 0.96 | 0.22 | 0.96 | 0.22 | 1.00 | 0.19 |
| | | | substitute, | | | | | | | | |
| | | | starch | | | | | | | | |
| | glycerol 2- | HMDB02520 | Chemical | 1.24 | 0.43 | 1.49 | 0.37 | 1.49 | 0.37 | 0.73 | 0.23 |
| | phosphate | | | | | | | | | | |
| | glycolate | HMDB00115 | Chemical | 1.14 | 0.30 | 0.97 | 0.38 | 0.97 | 0.38 | 0.76 | 0.14 |
| | (hydroxyacetate) | | | | | | | | | | |
| | hippurate | HMDB00714 | Benzoate | 0.94 | 0.26 | 1.14 | 0.39 | 1.14 | 0.39 | 0.78 | 0.16 |
| | | | metabolism | | | | | | | | |
| | pentobarbital | | Drug | 1.12 | 0.29 | 1.15 | 0.49 | 1.15 | 0.49 | 1.01 | 0.21 |

### DISCUSSION

The present report defines the major underlying non-lipid targets of dietary LC-O3PUFAs through the unbiased interrogation of spinal cord tissue metabolite levels. Dietary LC-O3PUFAs have a profound impact in the neural bioenergetics and antioxidant metabolomic profile. This was evidenced by marked and selective changes in the levels of carbohydrates, amino acids and nucleic acids.

Injury to the spinal cord leads to a complex cascade of pathophysiological processes. Multiple acute processes have been proposed that contribute to inflammation, cell death and dysfunction, and include ischemia, edema, cell membrane derangements, neurotransmitter and ionic imbalances, compromised energy metabolism, and production of free radicals. Prophylaxis and very early therapeutic interventions may be required to reduce the physical and psychological burden of disease on individuals at risk or afflicted by SCI.

### LC-O3PUFAs Confer Prophylaxis Against SCI

There are several clinical and occupational scenarios that present a significant risk of being affected by SCI. These situations include but are not limited to open repair for ruptured abdominal aortic aneurysm, thoracic endovascular aortic repair (TEVAR), amyotrophic lateral sclerosis, atherosclerosis, cerebral palsy, spina bifida, vitamin B12 deficiency, multiple sclerosis, iatrogenic ischemia, syringomyelia, spondylolysis, disc herniations, radiation toxicity, and tumors, sports, and military conflicts.

### LC-O3PUFAs Target Antioxidant Systems, Carbohydrate-Amino Acid Bioenergetics and Peptides in the Sham-Operated Spinal Cord

The neural metabolism slows considerably in the early phase of SCI, resulting in alterations to the energetic metabolism. Further, the production of reactive oxygen or nitrogen species that accompany the pathophysiology of SCI can lead to deleterious lipid and protein alterations. This increased oxidative and nitrative stress requires metabolic pathways to be redesigned to satisfy large demands for antioxidants. This study demonstrates that dietary LC-O3PUFAs can target the metabolism of carnosine and homocarnosine. These dipeptides are derived from histidine and GABA and represent major endogenous antioxidant/anti-inflammatory chemicals in the spinal cord. For example, administration of carnosine to SCI animals decreases immune cell infiltration, inducible nitric oxide synthase expression, pro-inflammatory cytokine expression, and apoptosis. Carnosine attenuates nociceptive responses in inflammatory pain models. These dipeptides could be partly responsible for the marked antinociceptive behaviors that are exhibited by animals consuming these long-chain fatty acids.

Glutamine is an important precursor of releasable glutamate and also one of the most effective substrates and modulators of glucose metabolism. The metabolism of glutamine is tightly regulated as it plays major roles in neurotransmission, immunomodulation, and glutathione synthesis and in SCI. Further, metabolic ratios evaluating chemical changes in glutamine-glutamate cycling may have prognostic value in neurotrauma and could help explain the state of gliosis and hyperexcitability often observed in SCI. Dietary LC-O3PUFAs target the glutamine-glutamate cycling even in sham animals. Because the LC-O3PUFA-rich diet did not alter the levels of glutamate in the spinal cord, this effect could reflect a reduced glutamine synthesis or accelerated glutamine recycling/catabolism. These findings imply that dietary LC-O3PUFAs modulate the expression of glutamine synthetase, which has been shown to protect from glutamate-induced excitotoxicity in SCI. The metabolism of tryptophan has also been implicated in the pathophysiology of SCI. The metabolism of tryptophan is geared towards the generation of kynurenines in the rats receiving LC-O3PUFA-rich diets. These metabolites are important regulators of mitochondrial homeostasis and oxidative stress, inflammation, and glutamate excitotoxicity.

The data shows that the levels of metabolic substrates and intermediates of glycolysis such as glucose, glucose-6-P, fructose, and sedoheptulose-7-P are significantly reduced by the LC-O3PUFA diet in the sham-operated animals. These observations together with our previous findings demonstrating that LC-O3PUFAs increase the levels of palmitoleate (a marker of de novo lipogenesis) suggest that these fatty acids reprogram the neural cell glucose bioenergetics. LC-O3PUFAs may stimulate energy production largely by oxidative phosphorylation via the tricarboxylic acid (TCA) cycle and pentose phosphate pathway (PPP) rather than glycolysis. This aneplerotic flux could have the advantage of diverting glycolytic intermediates into various biosynthetic pathways, which are essential for the synthesis of necessary macromolecules (i.e., amino acids, nucleosides and lipids required for assembling new cells). This idea is supported by recent findings showing that dietary LC-O3PUFAs reduce glycolysis by decreasing the expression of glycolytic enzymes. The reduced levels of selective amino acids with concomitant increases in the levels of urea and ornithine further support the strong influence of dietary PUFAs in modulating neural bioenergetics. Interestingly, the carbon skeleton of most of the amino acids that were reduced by the LC-O3PUFA diet can also feed into the TCA cycle and converted to pyruvate, acetyl CoA, acetoacetate, and succinyl CoA, suggesting a selective shunting of amino acids into the cycle. This observation indicates that the diet rich in LC-O3PUFAs may increase the protein and amino acid turnover and/or storage as fat and glycogen. Again, this could result in a more efficient system to handle excess amino nitrogen, which could be beneficial in situations in which neurons are starving or exposed to toxic levels of neurotransmitters such as during SCI.

Together, these observations lead us to conclude that the ability to regulate the neural cell bioenergetics and to increase and sustain stable levels of endogenous antioxidants may represent important mechanisms by which dietary LC-O3PUFAs confer prophylaxis against SCI.

### Administration of LC-O3PUFAs in the Diet Improves the Spinal Cord Redox Potential in the Injured Spinal Cord

Chronic oxidative damage after SCI results in neuroinflammation and maladaptive plasticity leading to sensorimotor abnormalities. The SH-containing tripeptide glutathione is effective in the protection of SH-carrying proteins against oxygen radicals and may thereby be especially important for neuroprotection. The glutathione metabolism is rapidly activated following SCI and its experimental depletion in SCI leads to appreciable worsening of neutrophil infiltration, lipid peroxidation, apoptosis, and loss of hind leg movement. Thus, the intracellular glutathione pool could be important for limiting oxidative stress-induced neuronal injury. Our data suggest that glutathione is a molecular target of dietary LC-O3PUFAs and could represent a key mechanism conferring protection in SCI rats and is inline with previous findings in rat hippocampal cultures.

Another finding that reflects an enhanced antioxidant system in rats fed with LC-O3PUFAs are the increased levels of sulfur amino acids. The increase in cystathionine and decrease of methionine observed in rats fed diets rich in LC-O3PUFAs suggests an increase in transulfuration pathway. This observation is supported by a study indicating that LC-O3PUFAs are capable of increasing the expression of cystathionine-γ-lyase (CSL), which catalyzes the splitting of cystathionine into cysteine and α-ketobutyrate.

The effect of the diet in increasing the levels of heme in the spinal cord was found, suggesting that dietary LC-O3PUFAs regulate the metabolism of heme-containing proteins such as hemoproteins. Hemoproteins contain a globin fold, the structural hallmark of the tissue hemoglobins, which enables the binding of oxygen, nitric oxide, or free radicals. The globin fold also enables the tissue hemoglobin to serve as either a facilitator of oxygen transport within a cell, a scavenger of nitric oxide, and/or an enzyme with peroxidase activity. Hemoproteins such as cytoglobin (CYGB) neuroglobin (Ngb) have been implicated in mediating neurorestorative responses in models of hypoxia-ischemia and spinal cord trauma.

### Dietary LC-O3PUFAs Increase the Levels of Glucose-Containing Oligosaccharides During the Acute Injury Phase

The diet rich in LC-O3PUFAs elevated the levels of oligosaccharides containing only glucose (i.e., maltose and maltotriose). Two potential mechanisms may be involved in this process: (1) the LC-O3PUFA diet facilitates the carbohydrate accumulation through glycogenesis or (2) the LC-O3PUFA diet may prevent the depletion of glycogen, a hallmark of ischemic damage. These mechanisms can facilitate energy production and offer metabolic alternatives to the damaged cord. For instance, LC-O3PUFAs could be important regulators of brain energy metabolism by affecting glucose utilization, the expression of the glucose transporter-1 (GLUT1), and the metabolism of glycans and glycoproteins, supporting the results described herein.

### LC-O3PUFAs Increase the Levels of Pyrimidines and Epigenetic Biomarkers

Brain phosphatide synthesis requires three circulating compounds: DHA, uridine, and choline. Interestingly, oral administration of these precursors increases the levels of phosphatides, synaptic proteins and number of dendritic spines in the brain.

This study shows that dietary intake of LC-O3PUFAs increased the levels of acetyllysine. It is thus reasonable to propose that this increased acetylation levels of lysine could reflect potential epigenetic mechanisms mediated by the diet. In fact, DHA has been reported to increase the acetylation of H3 and Bcl-2 levels, promoting gene expression and suggesting a mechanism for the neuroprotective roles of DHA.

In summary, dietary-essential LC-O3PUFAs accrete in the spinal cord cell membranes and alter multiple crucial metabolic pathways implicated in cell bioenergetics, signal transduction, gene expression, synaptic plasticity, and calcium regulation. Although DHA and EPA structure is an excellent target for lipid peroxidation and may function as free radical scavenger, LC-O3PUFA accretion in advance of injury may promote neural resilience against SCI by regulating the neuron-glia metabolic network. Accumulated LC-O3PUFAs can be released from cell membranes at the onset of injury and trigger the docosanoid pathway and DHA-derived messengers, including docosatrienes, resolvins, and neuroprotectins, such as neuroprotectin D1.

### CONCLUSIONS

Small-molecule profiling defined the underlying biologic state and distinctive non-lipid metabolic signatures associated with dietary LC-O3PUFA. The consumption of a diet rich in LC-O3PUFAs has a wide-ranging impact on the spinal cord metabolism. These changes may explain some of the observed differences in the phenotype of animals fed LC-O3PUFA-rich diets after SCI. Global neurometabolomic analyses uncovered the LC-O3PUFA metabolome as quite diverse and defined various metabolic pathways, including amino acid neurotransmitter systems and antioxidant defenses as its principal non-lipid targets in vivo. These changes demonstrate that LC-O3PUFAs are able to influence signaling networks beyond those associated with lipid metabolism. The rapid treatment of SCI animals with DHA and EPA has been shown to ameliorate several of the secondary biological responses that accompany the physical trauma. These protective effects can be expanded to prophylactic interventions, which may be a preventive measure to provide critical protection to individuals with known risk for SCI, including surgical patients, contact sports athletes, soldiers, and first responders. Although dietary LC-O3PUFAs may lack target specificity when compared to other therapeutics, diet may be more effective with respect to costs and safety concerns and deserve serious consideration in clinical applications.

A “patient” as used herein may refer to a human or a non-human mammal, e.g., a dog, a cat, a mouse, a rat, a cow, a sheep, a pig, a goat, a non-human primate or a bird, e.g., a chicken, as well as any other vertebrate or invertebrate.

An “effective amount” or a “therapeutically effective amount” as used herein refers to an amount of a therapeutic agent that is effective to relieve, to some extent, or to reduce the likelihood of onset of, one or more of the symptoms of a disease or condition (e.g., neuropathic pain), and includes curing the disease or condition. A prophylactically effective amount as used herein refers to an amount that is effective to prevent or delay the onset of one or more symptoms of a disease or condition (e.g., neuropathic pain), or otherwise reduce the severity of said one or more symptoms, when administered to a subject who does not yet exhibit symptoms of a disease or condition, but who is susceptible to, or otherwise at risk of, a particular disease or condition.

“Treat,” “treatment,” or “treating,” as used herein refers to administering a compound or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

Administration of the compounds disclosed herein or the pharmaceutically acceptable salts thereof can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly.

The compounds useful as described above can be formulated into pharmaceutical compositions for use in treatment of these conditions. Standard pharmaceutical formulation techniques are used, such as those disclosed in Remington's The Science and Practice of Pharmacy, 21st Ed., Lippincott Williams & Wilkins (2005), incorporated herein by reference in its entirety. Accordingly, some embodiments include pharmaceutical compositions comprising: (a) a safe and therapeutically effective amount of a compound described herein (including enantiomers, diastereoisomers, tautomers, polymorphs, and solvates thereof), or pharmaceutically acceptable salts thereof; and (b) a pharmaceutically acceptable carrier, diluent, excipient or combination thereof.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject compound is basically determined by the way the compound is to be administered.

The compositions described herein are preferably provided in unit dosage form. As used herein, a “unit dosage form” is a composition containing an amount of a compound that is suitable for administration to an animal, preferably mammal subject, in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort may be maximized as much as possible, although sometimes formulation considerations (e.g. drug stability) may necessitate less than optimal comfort. In the case that comfort cannot be maximized, the liquid may be formulated such that the liquid is tolerable to the patient for topical ophthalmic use. Additionally, an ophthalmically acceptable liquid may either be packaged for single use, or contain a preservative to prevent contamination over multiple uses.

For ophthalmic application, solutions or medicaments are often prepared using a physiological saline solution as a major vehicle. Ophthalmic solutions may preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

In a similar vein, an ophthalmically acceptable antioxidant includes, but is not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration.

In embodiments that include administering a combination of a compound described herein and another agent, the combination may be provided to caregivers as a mixture, or the caregivers may mix the two agents prior to administration, or the two agents may be administered separately.

The actual dose of the active compounds described herein depends on the specific compound, and on the condition to be treated; the selection of the appropriate dose is well within the knowledge of the skilled artisan.

Some embodiments include the combination of compounds, therapeutic agents, and/or pharmaceutical compositions described herein. In such embodiments, the two or more agents may be administered at the same time or substantially the same time. In other embodiments, the two or more agents are administered sequentially. In some embodiments, the agents are administered through the same route (e.g. orally) and in yet other embodiments, the agents are administered through different routes (e.g. one agent is administered orally while another agent is administered intravenously).

In summary, chronic neuropathic pain (CNP) is a frequent comorbidity following spinal cord injury (SCI) and often fails to respond to conventional pain management strategies. Preventive administration of docosahexaenoic acid (DHA) or consumption of a diet rich in omega-3 polyunsaturated fatty acids (O3PUFAs) confers potent prophylaxis against SCI and improves functional recovery. This dietary strategy provides significant antinociceptive benefits in rats experiencing SCI-induced pain. Rats were fed control chow or chow enriched with O3PUFAs for 8 weeks before being subjected to sham or cord contusion surgeries, continuing the same diets after surgery for another 8 more weeks. The paw sensitivity to noxious heat was quantified for at least 8 weeks post-SCI using the Hargreaves test. SCI rats consuming the preventive O3PUFA-enriched diet exhibited a significant reduction in thermal hyperalgesia compared to those consuming the normal diet. Functional neurometabolomic profiling revealed a distinctive deregulation in the metabolism of endocannabinoids (eCB) and related N-acyl ethanolamines (NAEs) at 8 weeks post-SCI. O3PUFAs consumption led to a robust accumulation of novel NAE precursors, including the glycerophospho-containing docosahexaenoyl ethanolamine (DHEA), docosapentaenoyl ethanolamine (DPEA), and eicosapentaenoyl ethanolamine (EPEA). The tissue levels of these metabolites were significantly correlated with the antihyperalgesic phenotype. In addition, rats consuming the O3PUFA-rich diet showed reduced sprouting of nociceptive fibers containing CGRP and decreased inositols levels and dorsal horn neuron p38 MAPK expression, well-established markers of pain. Collectively, these results support the importance of dietary O3PUFA in the treatment of SCI-mediated pain.

1. A method for treating or preventing neuropathic pain in a patient in need thereof, the method comprising administering to the patient a diet comprising a therapeutically or prophylactically-effect amount of one or more omega-3 polyunsaturated fatty acids.

2. The method of claim 1, further comprising administering to the patient an effective amount of an N-acylated ethanolamine precursor.

3. The method of claim 2, wherein the N-acylated ethanolamine precursor comprises one or more glycerophospho-containing docosahexaenoyl ethanolamine, glycerophosphocontaining docosapentaenoyl ethanolamine, and glycerophospho-containing eicosapentaenoyl ethanolamine.

4. The method of claim 1, further comprising administering to the patient an effective amount of an N-acylated ethanolamine.

5. The method of claim 4, wherein the N-acylated ethanolamine comprises one or more of docosahexaenoyl ethanolamine, docosapentaenoyl ethanolamine, and eicosapentaenoyl ethanolamine.

6. The method of claim 1, further comprising administering an additional therapeutic or prophylactic agent.

7. The method of claim 6, wherein the therapeutic or prophylactic agent comprises an opiate.

8. The method of claim 6, wherein the therapeutic or prophylactic agent comprises an anti-inflammatory agent.

9. The method of claim 6, wherein the therapeutic or prophylactic agent comprises a cell.

10. A dietary composition for the treatment or prevention of neuropathic pain, the composition comprising a therapeutically or prophylactically-effect amount of one or more omega-3 polyunsaturated fatty acids.

11. The composition of claim 10, wherein the composition produces an effective amount of an N-acylated ethanolamine compound.

12. The composition of claim 11, wherein the N-acylated ethanolamine compound comprises one or more of docosahexaenoyl ethanolamine, docosapentaenoyl ethanolamine, eicosapentaenoyl ethanolamine, glycerophospho-containing docosahexaenoyl ethanolamine, glycerophospho-containing docosapentaenoyl ethanolamine, and glycerophospho-containing eicosapentaenoyl ethanolamine, in a pharmaceutically acceptable carrier.

13. The composition of claim 10, wherein the composition produces an effective amount of an N-acylated ethanolamine precursor.

14. The composition of claim 13, wherein the N-acylated ethanolamine precursor comprises one or more glycerophospho-containing docosahexaenoyl ethanolamine, glycerophospho-containing docosapentaenoyl ethanolamine, and glycerophospho-containing eicosapentaenoyl ethanolamine, in a pharmaceutically acceptable carrier.

15. The composition of claim 10, further comprising an additional therapeutic or prophylactic agent.

16. The composition of claim 15, wherein the therapeutic or prophylactic agent comprises an opiate.

17. The composition of claim 15, wherein the therapeutic or prophylactic agent comprises an anti-inflammatory agent.

18. The composition of claim 15, wherein the therapeutic or prophylactic agent comprises a cell.
